SEQUENCE LISTING (11 sequences)

SEQ ID NO: 1 - protein, 164 aa, Spirodela polyrrhiza
MGKARKDLASSGNCGDDDESAEGSGRSFNNLKRKRSRTSAVRESPASRSSVYRGAYEEEEAAARAYDLAALKFWGHDTLLNFPLSTYQGEMEEMEGLSREEYISFIRRHHKNGKWEARIGRVFGNKYLYLGIYGDLSLSLYEKSIKAGRVSWGGKGSLDYFLLL

SEQ ID NO: 2 - DNA, 495 bp, Spirodela polyrrhiza
  source 1..495: mol_type = other DNA
atggggaaggcccggaaggacctggccagcagcggcaactgcggcgatgatgatgaatccgctgagggcagcggcaggagcttcaacaatctgaagaggaagaggagcaggacgagcgccgtcagggagtcgccggcgtcgcgcagctctgtctaccgaggagcctacgaagaggaggaagccgccgctcgtgcttatgatcttgccgcgttgaagttctgggggcatgacacccttctgaacttccccctgtcgacataccaaggagagatggaggagatggaagggcttagcagggaagaatatataagcttcataagaaggcaccataagaatggaaagtgggaagccaggattggcagagtcttcggcaacaagtatctctacctgggaatctacggtgatctctctctctctctctatgaaaagtccatcaaagccggccgggttagttggggagggaaggggagccttgactacttcttgcttctctaa

SEQ ID NO: 3 - DNA, 495 bp, synthetic construct
  source 1..495: mol_type = other DNA
atgggaaaggcccgtaaggaccttgcttcctccggcaactgcggtgatgacgacgagtcagccgaaggaagcgggcgctccttcaacaacttgaagcgaaagaggagtcgtacctccgcagtgcgcgagtcgcccgcttctagatcctcagtttaccgtggagcctacgaggaggaggaagctgccgccagggcttacgacctcgcagccttgaagttctggggccacgatacccttctcaatttcccacttagcacctaccaaggagagatggaggagatggaaggactatcccgcgaggagtacatcagttttattcgtagacatcacaagaacggtaaatgggaagctaggatcggacgcgtgttcggcaacaagtatttgtacctgggaatttacggggacctttccctctctttgtacgagaagtcaatcaaggccggacgtgtcagctggggtggcaagggatctcttgactacttcctcttgctttga

SEQ ID NO: 4 - protein, 335 aa, Persea americana
MGKSSKPNNNSSDNNSKSTTKVKRTRKSVPRESPTQRSSIYRGVTRHRWTGRYEAHLWDKNCWNETQNKKGRQGAYDDETAAAHAYDLAALKYWGQDTILNFPASTYEEELKEMEGQSKEEYIGSLRRKSSGFSRGVSKYRGVARHHHNGRWEARIGRVFGNKYLYLGTYATQEEAATAYDMAAIEYRGLNAVTNFDLSRYIKWLRPGSQNPNSNTDQNPNPSPSPSTSTSPNSDIGLGFLHHHSSGGGDGVPLPRAGGGALNLLLQSTKFKEMLERTSAVDSPSTTPEADNPPRCSFPDDIQTYFECEDPGVGDDVIFGDLNSFAAPLIEGEWD

SEQ ID NO: 5 - DNA, 1008 bp, Persea americana
  source 1..1008: mol_type = other DNA
atggggaaatcatcaaaacccaacaacaacagcagtgacaacaatagcaagtcgactacaaaggtgaaaagaacgagaaaaagcgtgccaagagaatcacccacacaacgtagctccatctacagaggtgtcacaaggcatcgatggacagggagatatgaagctcatttgtgggataaaaattgctggaatgagacccaaaacaagaaaggaagacaaggtgcttatgatgatgaaactgcagctgcacacgcatatgacttggcagcactgaagtactggggacaggacactatcctcaactttcctgcatctacatatgaggaagaactgaaagaaatggaaggacaatcaaaagaagaatacattggatccttgagaaggaaaagtagtggcttttccaggggtgtatcaaagtacagaggtgttgcaaggcatcatcacaatggaagatgggaagctcgcattggaagagtgtttgggaacaaatacctttatctgggaacctatgccacccaagaagaagcagccacagcttacgacatggcggcaattgagtacaggggacttaacgccgttacgaatttcgatctgagccgttacatcaaatggcttcgccccggctcacaaaaccctaattccaacactgaccaaaaccctaaccctagccctagccctagcactagcactagcccaaattctgatatagggctgggcttcctccaccaccatagctcaggcggcggagacggggtccctctgccccgtgccgggggcggcgccctcaacctactgctgcagtcgacgaaattcaaggagatgttggagaggacgtcggcggtggactccccgtcgacgacgccggaagcagacaacccgccgcggtgcagctttcccgacgacatacagacgtactttgagtgcgaggatccaggcgtgggggatgacgtcatcttcggcgatctgaactcgttcgcagcgcctttgatcgagggcgagtgggactag

SEQ ID NO: 6 - protein, 398 aa, Sorghum bicolor
MAKPRKNSAAANNNNSSSNGAGDLTPRAKPKRTRKSVPRESPTQRSSVYRGVTRHRWTGRFEAHLWDKNSWNESQNKKGKQVYLGAYDDEEAAARAYDLAALKYWGPDTILNFPASAYEGEMKGMEGQSREEYIGSLRRKSSGFSRGVSKYRGVARHHHNGRWEARIGRVFGNKYLYLGTYATQEEAAMAYDMAAIEYRGLNAVTNFDLSRYIKWLRPGAGGMAAAAAAAQNPHPMLGGLAQQLLLPPPADTTTTDGAGAAAFQHDHHGAEAFPLPPRTSLGHTPTTSALSLLLQSPKFKEMIQRTESGTTTTTTTTSSLSSSPPPTPSPSPPRRSPAPTQPPVQAAARDASPHQRGFPEDVQTFFGCEDTAGIDVEALFFGDLAAYATPAFHFEMDL

SEQ ID NO: 7 - protein, 1197 aa, Sorghum bicolor
ATGGCGAAGCCTCGCAAGAACAGCGCCGCCGCCAACAACAACAACAGCAGCAGCAACGGCGCCGGCGACCTGACGCCGCGCGCGAAGCCGAAGCGCACGCGGAAGAGCGTGCCCCGGGAGTCCCCCACGCAGCGCAGCTCCGTCTACCGCGGCGTCACACGGCACCGGTGGACGGGGAGGTTCGAGGCTCACCTGTGGGACAAGAACAGCTGGAACGAGTCCCAGAACAAGAAGGGCAAGCAAGTTTACCTCGGCGCGTACGACGACGAGGAGGCAGCGGCGCGGGCGTATGACTTGGCGGCATTGAAGTACTGGGGCCCCGACACCATCCTCAACTTCCCGGCGTCTGCATATGAAGGAGAGATGAAAGGAATGGAGGGCCAGTCCAGGGAAGAGTATATTGGATCCTTGAGGAGGAAAAGCAGTGGGTTCTCCAGAGGTGTCTCCAAATACCGAGGTGTCGCGAGACATCACCACAACGGGAGATGGGAGGCGAGGATCGGTCGTGTTTTCGGAAACAAGTATCTCTACCTCGGGACTTACGCGACGCAGGAGGAGGCGGCGATGGCGTACGACATGGCGGCGATCGAGTACCGTGGCCTGAACGCCGTCACAAACTTCGACCTCAGCCGCTACATCAAGTGGCTCCGCCCGGGCGCCGGCGGGATGGCAGCCGCGGCGGCGGCGGCGCAGAACCCGCACCCGATGCTGGGCGGCCTGGCGCAGCAGCTGCTGCTGCCGCCGCCGGCGGACACCACCACCACCGACGGCGCCGGCGCCGCGGCGTTCCAGCACGACCACCACGGCGCCGAGGCGTTCCCGCTGCCGCCCAGGACGTCGCTGGGCCACACGCCCACGACGTCGGCGCTCAGCCTGCTGCTGCAGTCGCCCAAGTTCAAGGAGATGATCCAGCGGACCGAGAGCGGCACCACCACCACTACCACCACGACATCGTCGTTGTCCTCGTCGCCGCCGCCCACGCCGTCCCCGTCCCCGCCGCGGCGGTCGCCAGCGCCGACGCAGCCGCCGGTGCAGGCGGCGGCCAGGGACGCCTCGCCGCACCAGCGCGGCTTCCCCGAGGACGTACAGACATTCTTCGGCTGCGAGGACACCGCGGGCATCGACGTGGAAGCCCTCTTCTTTGGCGACCTCGCCGCGTACGCGACGCCGGCGTTCCACTTCGAGATGGACTTGTAG

SEQ ID NO: 8 - protein, 345 aa, Arabidopsis thaliana
MFIAVEVSPVMEDITRQSKKTSVENETGDDQSATSVVLKAKRKRRSQPRDAPPQRSSVHRGVTRHRWTGRYEAHLWDKNSWNETQTKKGRQVYLGAYDEEDAAARAYDLAALKYWGRDTILNFPLCNYEEDIKEMESQSKEEYIGSLRRKSSGFSRGVSKYRGVAKHHHNGRWEARIGRVFGNKYLYLGTYATQEEAAIAYDIAAIEYRGLNAVTNFDISRYLKLPVPENPIDTANNLLESPHSDLSPFIKPNHESDLSQSQSSSEDNDDRKTKLLKSSPLVAEEVIGPSTPPEIAPPRRSFPEDIQTYFGCQNSGKLTAEEDDVIFGDLDSFLTPDFYSELNDC

SEQ ID NO: 9 - DNA, 1416 bp, Arabidopsis thaliana
  source 1..1416: mol_type = other DNA
ctaaatggtttgattacgattaggaattttaataaatgagaaatctaattacagaagtacaaagagggccgtagcaaaggaaatcttttaaagcaatagcaattaaaaaggctaaaaaaaataaaacggaaaattataattttaaatactcaaccatttctctttatagtttccttataaaacaaaaacaaaaaccattccatcaatttctcatccttgttgttttcttcttcttcttcttcctcttcttttatgttcatcgccgtcgaagtttctccggtaatggaagacatcacacggcagagcaaaaaaacttcggttgagaatgaaaccggcgatgatcagtcagcaacatcagtagtccttaaagctaaacgcaaacgccgatcgcaaccacgagacgctccaccccaacgtagctccgtccatagaggcgtcacaaggcatcgatggactggaaggtacgaagcacatttgtgggataagaatagttggaacgaaactcagaccaagaaaggaagacaagtatatttaggggcatatgacgaggaagatgcagcagcacgtgcctacgacttagcagcattgaaatattggggacgagacaccatcttgaacttccctttgtgtaattatgaagaagacatcaaagaaatggaaagccagtcaaaggaagagtatattggatctttgagaagaaaaagtagtgggttttcacgaggtgtatcaaaatacagaggcgttgcaaagcatcaccacaatgggagatgggaagctcgaatcggaagagtgtttggcaataaatatttataccttggaacttacgcgacgcaagaagaagcagctatagcgtacgatatcgcagctatcgagtaccgtggactcaacgccgttactaacttcgacatcagccgttatctgaaactcccggtgccggagaaccctatcgataccgcgaataatctcctcgagagtccgcattctgatcttagcccatttataaaacctaaccacgagtctgacttatcacagagtcaatcttcgtcagaggacaacgatgatcggaaaacaaagctcttgaagtcgtcacctttagtggcagaggaggtaatcggaccatcgacgccacctgagattgctccgcctcgtcggagcttcccggaagatatccagacgtatttcgggtgtcaaaactccggcaagttaacggcggaggaagatgatgttatcttcggtgatttagattctttccttacgcctgatttctacagcgagttaaatgattgctaaagtgttgttcttctgataagttttgttttttagttgttcagaatctcggttgtgaaaatcaacattgacacatcgattattctttcttgtgacaatcttatataataaagtttgaatctttttaac

SEQ ID NO: 10 - protein, 164 aa, Spirodela polyrrhiza
MGKARKDLASSGNCGDDDESAEGSGRSFNNLKRKRSRTSAVRESPASRSSVYRGAYEEEEAAARAYDLAALKFWGHDTLLNFPLSTYQGEMEEMEGLSREEYISFIRRHHKNGKWEARIGRVFGNKYLYLGIYGDLSLSLYEKSIKAGTVSWGGKGSLDYFLLL

SEQ ID NO: 11 - protein, 285 aa, Arabidopsis thaliana
MFIAVEVSPVMEDITRQSKKTSVENETGDDQSATSVVLKAKRKRRSQPRDAPPQRSSVHRGVTRHRWTGRYEAHLWDKNSWNETQTKKGRQVYLGAYDEEDAAARAYDLAALKYWGRDTILNFPLCNYEEDIKEMESQSKEEYIGSLRRKSSGFSRGVSKYRGVAKHHHNGRWEARIGRVFGNKYLYLGTYATQEEAAIAYDIAAIEYRGLNAVTNFDISRYLKLPVPENPIPEIAPPRRSFPEDIQTYFGCQNSGKLTAEEDDVIFGDLDSFLTPDFYSELNDC

(19) United States
(12) Patent Application Publication
(10) Pub. No.: US 2025/0002928 A1
(43) Pub. Date: Jan. 2, 2025

(54) ENGINEERING PLANTS TO ENHANCE TOLERANCE TO FLUE GAS DESULFURIZATION (FGD) WASTEWATER, SALT STRESS AND TRIACYLGLYCEROLS (TAGS) PRODUCTION

(71) Applicant: West Virginia State Universsity Research and Development Corporation, Institute, WV (US)

(72) Inventors: Sanju A. Sanjaya, Charleston, WV (US); Bagyalakshmi Muthan, Charleston, WV (US)

(21) Appl. No.: 18/757,072

(22) Filed: Jun. 27, 2024

Related U.S. Application Data
(60) Provisional application No. 63/523,582, filed on Jun. 27, 2023.

(51) Int. Cl. C12N 15/82; C07K 14/415

(52) U.S. Cl. CPC C12N 15/8271; C07K 14/415; C12N 15/8222; C12N 15/8273

(57) ABSTRACT

Described herein are expression cassettes, plant cells, plant seeds, plants, and methods for generating a transgenic plant seed or plant that is tolerant to heavy metal stress, salt stress, or a combination thereof. The expression cassette comprises a promoter operably linked to a nucleic acid segment encoding a polypeptide for SpWRI3.

### PRIORITY

This application claims the benefit of priority of the filing date of U.S. provisional application No. 63/523,582, filed on Jun. 27, 2023, the disclosure of which is incorporated by reference herein in its entirety.

### STATEMENT OF GOVERNMENT SUPPORT

This invention was made with government support under DE-FE0027893 awarded by the Department of Energy, W911NF-15-1-0055 awarded by the Department of Defense, 2018-38821-2811, 2018-38821-27753, 2020-38821-31122 and 2022-67014-37050 awarded by the U.S. Department of Agriculture's National Institute of Food and Agriculture and DE-SC0024647 awarded by the Department of Energy. The government has certain rights in the invention.

### INCORPORATION OF SEQUENCE LISTING BY REFERENCE

This application contains a sequence listing. It has been submitted electronically as an XML file titled “6222001US1.xml.” The sequence listing is 16,990 bytes in size and was created on Jun. 25, 2024. It is hereby incorporated by reference in its entirety.

### BACKGROUND OF THE INVENTION

Crops increasingly experience heavy metal (HM) stress due to anthropogenic activities such as excessive use of pesticides and fertilizer in agriculture, improper treatment of industrial waste, and intensive mining. These activities compromise the quality of soil and freshwater resources and reduce world agricultural production. HMs are readily soluble in water and easily absorbed by plants, resulting in entry to the food web, and, later, biomagnified levels of HMs are consumed by humans. Plants depend on HMs as micronutrients required as cofactors for biochemical reactions and other physiological processes but become toxic in excess.

One event in HM toxicity is the production of reactive oxygen species (ROS) by ROS-active metals and damage to cellular membranes, resulting in the production of free fatty acids (FFAs), fatty acid esters, and derivatives such as phosphatidic acid (PA), phosphatidylinositol (PI), sphingolipids, oxylipins, N-acylethanolamines, and others. These are toxic to cells, and their accumulation can disturb cellar signaling pathways and induce oxidative stress and subsequent cell death, known as lipotoxicity. Plants have evolved a mechanism to overcome these undesired effects by producing enzymes responsible for detoxifying ROS and sequestration of toxic lipid intermediates such as FFAs from the damaged membrane into triacylglycerols (TAGs) and preventing cellular toxicity. TAGs in photosynthetic cells may play a role in the sequestration of toxic lipid intermediates from the damaged membrane and prevent cellular toxicity from the FFAs.

In plants, chloroplast stroma is the primary site of fatty acid biosynthesis for producing membrane lipids such as monogalactosyldiacylglycerol (MGDG), digalactosyldiacylglycerol (DGDG), phosphatidylglycerol (PG), and sulfoquinovosyldiacylglycerol (SQDG); these are the main structural components of photosynthetic membranes. MGDG and DGDG represent 70-80% of chloroplast lipids. Membrane lipids are major targets of environmental stresses and undergo changes in their synthesis and composition in adaptation to stress. In the recent past, considerable progress has been made toward understanding membrane lipid modifications in HM-stressed plants. For example, HM treatment induced an alteration in lipid synthesis, changes in lipid composition, and fatty acid desaturation in tomatoes; the freshwater vascular plant Hydrilla verticillate, and Arabidopsis. In rice, HM exposure inhibited photosynthetic pigment development and reduced crop yield. Synthesis of DGDG and MGDG was reduced in stressed Arabidopsis and wheat, respectively. Membrane phospholipids are also primary degradation targets of stress; using lipidomic approaches in Arabidopsis revealed that altered expression of phospholipases improved freezing tolerance by modification in lipid species. Different stresses induce the production of TAGs in leaf tissues due to membrane lipid remodification as a survival strategy against lipotoxicity. In contrast to terrestrial plants, aquatic plants are completely exposed to polluted water, and also, many aquatic plants are accumulators of HM.

### SUMMARY

Described herein are plants, plant cells, and plant seeds that provide enhanced tolerance to heavy metals commonly found in industrial wastewater and reclaimed coal mine soils for improved crop production in marginal soils. The nucleic acids, expression cassettes, plants, seeds, and methods described herein can be used to improve the tolerance of the plants to salt stress and reduce the accumulation levels of malondialdehyde (MDA), H₂0₂, and superoxide radicals in the transgenic plants. The nucleic acids, expression cassettes, plants, seeds, and methods described herein can be used to increase the accumulation of triacylglycerol (TAG) enriched with omega-3 fatty acids and carbohydrates in transgenic plants. Methods of cultivating such plant seeds and plants are also described herein that include, for example, harvesting the seeds or vegetative tissues of the plants. Such methods can also include isolating the TAG enriched with omega-3 fatty acids for use in nutritional supplements, food preparation, or aquatic or animal feed.

For example, plant cells, plant seeds, and plants are described herein that include an expression system comprising at least one expression vector comprising a nucleic acid segment encoding a WRINKLED transcription factor 3 from Spirodella polyrhiza (S. polyrhiza), (SpWRI3). As described herein the SpWRI3 polypeptide transcription factor is a fatty acid metabolism gene found to be highly upregulated in S. polyrhiza cultivated in flue gas desulfurization (FGD) wastewater containing trace concentrations of heavy metal contaminants and high levels of dissolved solids.

In addition, methods are described herein that include comprising growing a plant seed or plant comprising an expression system comprising a heterologous promoter operably linked to a nucleic acid segment encoding a SpWRI3 polypeptide transcription factor, to thereby produce a mature plant.

### DESCRIPTION OF THE FIGURES

FIGS. 1A-1C the physiological response of S. polyrhiza to FGD wastewater. (A) Morphological response of S. polyrhiza to FGD wastewater and control condition. (B) Relative chlorophyll content. (C) Dry biomass of S. polyrhiza under four exposure times 0 h, 24 h, 48 h, and 72 h on FGD wastewater and tap water conditions. t test significant at *P<0.01, ***P<0.0001 versus plants in the T condition [chlorophyll (n=4); dry weight (n=200 plants) average±SD].

FIGS. 2A-2F are transmission electron microscopy (TEM) analysis of S. polyrhiza fronds treated in tap water and FGD wastewater conditions. (A) to (C) Fronds treated in tap water condition at 24 h, 48 h, and 72 h. (D) to (F) Fronds treated in FGD wastewater conditions at 24 h, 48 h, and 72 h. OD: Oil droplets; Ch: Chloroplasts; S: Starch granules; PS: Plastoglobules; T: Tap water; F: FGD wastewater Bars=0.5 m.

FIGS. 3A-3B are graphs demonstrating that exposure of S. polyrhiza to tap water and FGD wastewater induced a considerable amount of triacylglycerol (TAG). (A) Relative levels of total TAG (normalized MS signal per dry mass) in neutral lipid extracts of control (0 h medium grown), tap water, and FGD wastewater treated plantlets at 24 h, 48 h, and 72 h. (B) Relative levels of total diacylglycerol (DAG) (normalized MS signal per dry mass) in neutral lipid extracts of control (0 h medium grown), tap water, and FGD wastewater treated plants at 24 h, 48 h, and 72 h. t test significant at *P<0.05, ***P<0.001 in the FGD wastewater condition versus plantlets in the tap water condition (n=3; average±SD).

FIGS. 4A-4H are graphs depicting altered polar lipid composition in S. polyrhiza grown in 0 h nutrient medium and exposed to tap water and FGD wastewater at 24 h, 48 h, and 72 h. (A) Total polar lipids. (B) Digalactosyldiacylglycerol (DGDG). (C) Monogalactosyldiacylglycerol (MGDG). (D) Phosphatidylcholine (PC). (E) Phosphatidylethanolamine (PE). (F) Lysophosphatidylethanolamine (LysoPE). (G) Phosphatidylinositol (PI). (H) Phosphatidic acid (PA). t test significant at *P<0.05, **P<0.01, ***P<0.001 in the FGD wastewater condition versus plantlets in the tap water condition (n=3; average±SD).

FIGS. 5A-5H are graphs depicting altered unsaturation indices in lipids of S. polyrhiza grown in 0 h nutrient medium and exposed to tap water and FGD wastewater at 24 h, 48 h, and 72 h. (A) Total polar lipids. (B) Phosphatidylglycerol (PG). (C) Phosphatidylcholine (PC). (D) Phosphatidylethanolamine (PE). (E) Phosphatidylinositol (PI). (F) Phosphatidic acid (PA). (G) Diacylglycerol (DAG). (H) Triacylglycerol (TAG). t test significant at *P<0.05, **P<0.01, ***P<0.001 in the FGD wastewater condition versus plantlets in the tap water condition (n=3; average±SD).

FIGS. 6A-6F shows the changes in radiolabeling distribution among different lipids under T (Tap water) and F (FGD wastewater) conditions in S. polyrhiza. (A) Total lipids were separated by TLC and radioactivity associated with individual lipids was quantified by liquid scintillation counting. (B) Phosphatidylcholine (PC). (C) Phosphatidylethanolamine (PE). (D) Digalactosyldiacylglycerol Phosphatidylinositol/sulfoquinovosyl diacylglycerol (DGDG/PI/SQDG). (E) Phosphatidylglycerol (PG). (F) Monogalactosyldiacylglycerol (MGDG), (G) Triacylglycerol (TAG). Data are means of three independent biological replicates with SD. Statistically significant differences between plantlets treated in tap water and FGD wastewater conditions were determined by the two-tailed Student's t test (*P<0.05, **P<0.01, ***P<0.001).

FIGS. 7A-7I are graphs depicting FGD wastewater treatment alters duckweed frond cuticular wax composition. A-G. Frond wax class content (μg per mg⁻¹ of dry mass) from surface lipid extracts of control (0 h medium grown), tap water, and FGD wastewater-grown plants after 24 h, 48 h, and 72 h of growth in each respective treatment. H, I. 24:0 and 26:0 1-alcohol content from FGD wastewater and tap water-treated fronds. Data are presented as mean values±SD (n=3). Significant differences between FGD wastewater and tap water treated plantlets were determined at each time point using a Student's t-test. *P<0.05, **P<0.01, ***P<0.001.

FIGS. 8A-8C illustrate that starch granules are larger in S. polyrhiza exposed to FGD wastewater. (A) Carbohydrate content of the control (0 h medium grown), tap water, and FGD wastewater treated plantlets. (B) and (C) TEM analysis of S. polyrhiza fronds from 72 h tap water and FGD wastewater conditions, respectively. t test significant at *P<0.01, ***P<0.0001 versus plantlets in the tapwater condition (n=6; average±SD). Ch: Chloroplast; S: Starch granule; P: Plastoglobules. Bars=0.2 and 1 m.

FIGS. 9A-9B illustrate the differentially expressed genes related to lipid metabolism. (A) and (B) Venn diagram of lipid metabolism-related genes upregulated and downregulated in 24 h, 48 h, and 72 h tap water and FGD wastewater treated S. polyrhiza. Differentially expressed genes were identified between the treatment FGD wastewater and tap water across the three sampling time points (i.e., 24, 48, and 72 h), respectively.

FIGS. 10A-10B illustrate the differentially expressed genes related to starch metabolism. (A) and (B) Venn diagram of starch metabolism-related genes upregulated and downregulated in 24 h, 48 h, and 72 h tap water and FGD wastewater treated S. polyrhiza. Differentially expressed genes were identified between the treatment FGD wastewater and tap water across the three sampling time points (i.e., 24, 48, and 72 h), respectively.

FIGS. 11A-11F illustrate the physiological characterization of Col-0 and transgenic Arabidopsis plants overexpressing SpWRI3 (Sp11g00856) in response to flue gas desulfurization (FGD) wastewater. (A) Col-0 and independent transgenic Arabidopsis lines (13-2, 5-2, and 22-1) were grown in a hydroponic setup (liquid half MS) before the FGD wastewater treatment. (B) 72 h after the FGD wastewater treatment of Col-0 and transgenic Arabidopsis lines. (C) DAB (3,3-diaminobenzidine) (for H2O2) staining of seedlings from Col-0 and SpWRI3-overexpressing Arabidopsis line 22-1 were treated with FGD wastewater for 72 h. Bars, 500 m. (D) NBT (for superoxide radicals), staining of leaves from Col-0, and SpWRI3-overexpressing Arabidopsis line 22-1 were treated with FGD wastewater for 72 h. Bars, 500 m. (E) Reactive oxygen species (ROS) and malondialdehyde (MDA) accumulation in Col-0 and transgenic Arabidopsis lines in response to 72 h FGD wastewater treatment. (F) Quantification of reduced GSH in Col-0 and transgenic Arabidopsis lines in response to 72 h FGD wastewater treatment t-test significant at ***P<0.001 in the transgenic lines versus Col-0 plants (n=3; average±SD).

FIGS. 12A-12B are (A) Schematic overview of the degradation of monogalactosyldiacylglycerol (MGDG) and digalactosyldiacylglycerol (DGDG) by FGD wastewater-induced heavy metal (HM) stress and subsequent incorporation of 18:3 free fatty acids into TAG. (B) Schematic representation of starch degradation, carbon recycling, and increased starch granule size. Triacylglycerol (TAG); S. polyrhiza WRINKLED3 (SpWRI3) (Sp11g00856); plastid lipase2 (PLIP2; Sp01g01058); beta-amylase 1 (BAM1; Sp14g00110); beta-amylase 5 (BAM5; Sp17g00401); water dikinase 1 (GWD1; Sp08g00310); alpha-amylase 2 (AMY2; Sp09g00344); alpha-amylase 3 (AMY3; Sp13g00637); hexokinase 1 (HXK1/GIN2; Sp06g00791) and Glc6P/Pi translocator 2 (GPT2; Sp13g00970) and AGPase large subunit 2 (APL2; Sp19g00510).

FIG. S1 provides a graph of altered PC to PE ratio of S. polyrhiza grown in 0 h nutrient medium and exposed to tap water and FGD wastewater at 24 h, 48 h, and 72 h. t test significant at **P<0.01, ***P<0.001 in the F condition versus plants in the T condition (n=3; average±SD).

FIG. S2 provides graphs showing that FGD wastewater (F) treatment alters S. polyrhiza frond cuticular wax composition. Frond wax composition (μg per mg⁻¹ of dry mass) from surface lipid extracts of control (0 h medium grown), tap water (T), and FGD wastewater treated plants at 24 h, 48 h, and 72 h. The Inset line graph shows total wax amounts throughout the time course. Data are presented as mean values±SD (n=3). Significant differences between FGD wastewater and tap water-treated plantlets were determined at each time point using a Student's t-test. *P<0.05, **P<0.01, ***P<0.001. MAGs: Monacylglycerols.

FIG. S3 provides graphs of validation of differential lipid metabolism gene expression data by quantitative RT-PCR in 0 h, 24 h, 48 h, and 72h T (tap water) and F (FGD wastewater) treated S. polyrhiza. Sp16g00101: Long-Chain Acyl-CoA Synthetase; Sp09g00263: Pyruvate Dehydrogenase alpha subunit; Sp11g00856: WRINKLED3 transcription factor 3; Sp14g00361: Lysophosphatidic acid acyltransferase CGI-58; Sp01g00757: Triacylglycerol Lipase (SDP1); Sp01g01058: Plastid lipase (PLIP2); Sp18g00052: Ketoacyl-ACP Synthase II; Sp14g00739: Glycerol-3-Phosphate Acyltransferase; Sp01g01469: Linoleate Desaturase; Sp04g01109: Ketoacyl-CoA Reductase; Sp07g00488: CER7 Protein involved in wax synthesis; Sp12g00731: WRINKLED4 transcription factor.

FIG. S4 provides graphs of validation of differential carbohydrate metabolism gene expression data by quantitative RT-PCR in 0 h, 24 h, 48 h, and 72h tap water (T) and FGD wastewater (F) treated S. polyrhiza. Sp19g00510: AGPase large subunit 2; Sp07g00971: Isoamylase 3 (ISA3); Sp09g00344: Alpha amylase 2 (AMY2); Sp14g00110: Beta-amylase 1 (BAM1); Sp18g00726: Starch synthase 2 (SS2); Sp05g00808: AGPase small subunit 1; Sp01g01820: Similar to PWD; Sp08g00534: Starch synthase 1.

FIG. S5 provides multiple sequence alignment of S. polyrhiza WRINKLED3 (SpWRI3, Sp11g00856) and Arabidopsis thaliana WRINKLED3 (AtWRI3, At1g16060) (SEQ ID NOs: 10 and 11).

FIGS. S6A-S6B demonstrates ectopic expression of SpWRI3 (Sp11g00856) in Arabidopsis (A) Schematic representation of binary vectors consisting of SpWRI3. (B) Validation of expression of Sp11g00856 (SpWRI3) in transgenic Arabidopsis by quantitative RT-PCR. #13-2, #15-2, and #22-1 are independent transgenic Arabidopsis plants. LB: left border; RB: right border; HptII: hygromycin phosphotransferase II; 355: cauliflower mosaic virus 35S promoter; NT. Nos terminator; UBQ10. Arabidopsis Ubiquitin 10 promoter.

FIGS. S7A-S7B provide (A) Analysis of TAG in 3-week-old Col-0 and transgenic Arabidopsis plants overexpressing SpWRI3 (Sp11g00856) in response to 72 h flue gas desulfurization (FGD) wastewater treatment by a thin-layer chromatogram (TLC). Lipids were visualized with iodine vapor. Col-0 and independent transgenic Arabidopsis lines (#13-2, #15-2, and #22-1) were grown in a hydroponic setup consisting of liquid half-strength MS (control) and FGD wastewater for 72 h under normal growth conditions. (B) Relative contents of TAG in Col-0 and transgenic lines were quantified by ImageJ software. TAG: Triacylglycerol; FFA: Free fatty acids; DAG: Diacylglycerol. t-test significant at ***P<0.001 in the transgenic lines versus Col-0 plants treated with FGD wastewater (n=3; average±SD).

FIG. S8 provides analysis of starch accumulation in 3-week-old Col-0 and transgenic Arabidopsis plants overexpressing SpWRI3 (Sp11g00856) in response to 72 h flue gas desulfurization (FGD) wastewater treatment. Lugol staining of whole Arabidopsis plants grown in a hydroponic setup consisting of liquid half-strength MS (control) and FGD wastewater for 72 h under normal growth conditions. To compare the intensities of the staining, identical photographic lighting and exposure conditions were used for all the photos. C: Control (half MS), F: FGD wastewater Bars, 500 μm.

### DETAILED DESCRIPTION

Described herein are plants, plant cells, and plant seeds that provide enhanced tolerance to heavy metal (HM) stress, salt stress, or a combination thereof and enhanced accumulation of triacylglycerol (TAG) enriched with omega-3 fatty acids and carbohydrates. Methods are described herein for providing transgenic plants suitable for phytoremediation of heavy metals (HM) in wastewater or bioproduct production (e.g., biodiesel or starch). For example, flue gas desulfurization (FGD) wastewater contains trace concentrations of HM contaminants and high levels of dissolved solids. As described herein, expression of S. polyrhiza WRINKLED transcription factor 3 (“SpWRI3”) in plants can be used to provide enhanced tolerance to FGD wastewater by minimizing membrane damage and reducing levels of malondialdehyde (MDA), reactive oxygen species such as hydrogen peroxide (H₂0₂), and superoxide radical (O₂⁻) production in the plants. The SpWRI3 transcription factor can be used in developing crops tolerant to heavy metals commonly found in industrial wastewater and reclaimed coal mine soils for improved crop production in marginal soils.

Unlike terrestrial angiosperms, duckweed (Spirodela polyrhiza L.), a group of freshwater aquatic plants, grow directly in water, and their response to heavy metal (HM) stress is distinct. Plants accumulate metabolites, including lipids and carbohydrates, when they experience HM stress, but the mechanism by which plants balance the levels of these metabolites and associated gene networks in mediating HM stress was previously unknown. Flue gas desulfurization (FGD) wastewater-induced HM stress is described herein to lower chlorophyll content, inhibit growth and membrane lipid synthesis, and stimulate membrane lipid degradation, leading to the accumulation of triacylglycerol (TAG) and carbohydrates in S. polyrhiza. Using a time-course ¹⁴C-acetate pulse-chase radiolabeling assay in HM stressed plants, degraded products of monogalactosyldiacylglycerol (MGDG), i.e., mainly polyunsaturated fatty acids (18:3), are shown herein to be incorporated into TAGs and subsequently sequestrated into lipid droplets. Also, HM stress considerably increased the accumulation of carbohydrates and enlarged starch granule size in S. polyrhiza. HM stress induced an array of enzymes or genes involved in the early events in fatty acid synthesis, beta-oxidation, and degradation of lipids, including membrane lipids and TAG. HM stress significantly downregulated most genes involved in cuticular wax synthesis and altered the expression of genes involved in starch biosynthesis.

A differential gene expression study found upregulation of transcription factor WRINKLED3 (SpWRI3; Sp11g00856) in FGD wastewater-treated plants. Ectopic expression of SpWRI3 increases tolerance to FGD wastewater in transgenic Arabidopsis. Under stress, SpWRI3 overexpression enhanced the accumulation of glutathione and reduced MDA contents in transgenic plants. These results indicate that the SpWRI3 transcription factor participates in FGD wastewater-induced HM stress tolerance in S. polyrhiza. The results provide a basis for HM stress tolerance improvement of plants for industrial application.

Spirodella polyrhiza

Spirodella polyrhiza, a member of the duckweed family, is among the smallest and simplest monocotyledonous flowering plants found floating in nutrient-rich aquatic environments worldwide. Its aquatic habit, fast growth, high biomass, and smallest genome size (158 Mbp) in the duckweed family make it amenable to basic and applied research. Because of its robust growth habitat, physiology, and genetics, S. polyrhiza can be used in phytoremediation, animal, and human feed, and biofuel production. Furthermore, advances in S. polyrhiza genetics and genomics provided insight into the molecular mechanisms of high production of starch and protein and stress tolerance. A study has analyzed the fatty acids and TAG levels in thirty (30) duckweed species and confirmed that TAG accumulation levels in duckweed are comparable to those in leaves of terrestrial plants. Recently, genetic engineering strategies have been established for Lemna japonica to accumulate TAG for industrial applications. However, S. polyrhiza is not fully exploited as a potential plant system for producing bioproducts because of an unclear understanding of TAG/oil biosynthesis and induction mechanisms. Thus, detailed knowledge of the change in fatty acid biosynthesis, TAG, and carbohydrate metabolism in response to HM stress will help in the design of S. polyrhiza or other aquatic plants to survive in harsh conditions as potential bioreactors.

As described herein, S. polyrhiza was selected for time-course experiments to determine the biochemical changes in response to FGD wastewater-derived HM stress. Lipidomics and transcriptomics were used to elucidate the dynamic changes in lipids and gene expression in response to HM stress tolerance mechanisms in S. polyrhiza. Results described herein suggest that HM stress promotes the degradation of chlorophyll lipids and the accumulation of TAGs via active membrane lipid modification in S. polyrihiza. Transcriptomic data suggest that TAGs and starch biosynthesis are independent, unlike those of unicellular photosynthetic organisms. The degradation of starch presumably provides carbon flux to support cellular metabolism under HM stress conditions. Additionally, using differential gene expression studies, one of the top upregulated genes SpWRI3 was identified. Using transgenic Arabidopsis, SpWRI3 was characterized for its role in tolerance to FGD wastewater-induced heavy metal stress. The results provide novel insights into the underlying mechanisms of the HM stress response and a valuable foundation for improving phytoremediation or bioproduct (biodiesel or starch) in S. polyrihiza.

### SpWRI3

SpWRI3 (Sp11g00856) is a member of the APETALA2/ETHYLENE-RESPONSIVE ELEMENT BINDING (AP2/EREB) transcription factor family.

Transcription factors in this family, including WRI1, WRI2, and WRI3, behave as transcriptional activators of the fatty acid biosynthetic pathway. A SpWRI3 protein sequence from S. polyrihiza is shown below as SEQ ID NO: 1.

MGKARKDLASSGNCGDDDESAEGSGRSFNNLKRKRSRTSAVRESPASRS
SVYRGAYEEEEAAARAYDLAALKFWGHDTLLNFPLSTYQGEMEEMEGLS
REEYISFIRRHHKNGKWEARIGRVFGNKYLYLGIYGDLSLSLYEKSIKA
GRVSWGGKGSLDYFLLL

A nucleotide sequence that encodes the SpWRI3 protein with SEQ ID NO 1 is shown below as SEQ ID NO: 2:

ATGGGGAAGGCCCGGAAGGACCTGGCCAGCAGCGGCAACTGCGGCGATG
ATGATGAATCCGCTGAGGGCAGCGGCAGGAGCTTCAACAATCTGAAGAG
GAAGAGGAGCAGGACGAGCGCCGTCAGGGAGTCGCCGGCGTCGCGCAGC
TCTGTCTACCGAGGAGCCTACGAAGAGGAGGAAGCCGCCGCTCGTGCTT
ATGATCTTGCCGCGTTGAAGTTCTGGGGGCATGACACCCTTCTGAACTT
CCCCCTGTCGACATACCAAGGAGAGATGGAGGAGATGGAAGGGCTTAGC
AGGGAAGAATATATAAGCTTCATAAGAAGGCACCATAAGAATGGAAAGT
GGGAAGCCAGGATTGGCAGAGTCTTCGGCAACAAGTATCTCTACCTGGG
AATCTACGGTGATCTCTCTCTCTCTCTCTATGAAAAGTCCATCAAAGCC
GGCCGGGTTAGTTGGGGAGGGAAGGGGAGCCTTGACTACTTCTTGCTTC
TCTAA

A codon-optimized nucleotide sequence that encodes the SpWRI3 protein with SEQ ID NO: 1 is shown below as SEQ ID NO: 3:

ATGGG AAAGGCCCGT AAGGACCTTG CTTCCTCCGG CAACTGCGGT
GATGACGACG AGTCAGCCGA AGGAAGCGGG CGCTCCTTCA
ACAACTTGAA GCGAAAGAGG AGTCGTACCT CCGCAGTGCG
CGAGTCGCCC GCTTCTAGAT CCTCAGTTTA CCGTGGAGCC
TACGAGGAGG AGGAAGCTGC CGCCAGGGCT TACGACCTCG
CAGCCTTGAA GTTCTGGGGC CACGATACCC TTCTCAATTT
CCCACTTAGC ACCTACCAAG GAGAGATGGA GGAGATGGAA
GGACTATCCC GCGAGGAGTA CATCAGTTTT ATTCGTAGAC
ATCACAAGAA CGGTAAATGG GAAGCTAGGA TCGGACGCGT
GTTCGGCAAC AAGTATTTGT ACCTGGGAAT TTACGGGGAC
CTTTCCCTCT CTTTGTACGA GAAGTCAATC AAGGCCGGAC
GTGTCAGCTG GGGTGGCAAG GGATCTCTTG ACTACTTCCT
CTTGCTTTGA

A WRI3 protein sequence from Persea americana (avocado) is shown below as SEQ ID NO: 4.

MGKSSKPNNNSSDNNSKSTTKVKRTRKSVPRESPTQRSSIYRGVTRHRWT
GRYEAHLWDKNCWNETQNKKGRQGAYDDETAAAHAYDLAALKYWGQDTIL
NFPASTYEEELKEMEGQSKEEYIGSLRRKSSGFSRGVSKYRGVARHHHNG
RWEARIGRVFGNKYLYLGTYATQEEAATAYDMAAIEYRGLNAVTNFDLSR
YIKWLRPGSQNPNSNTDQNPNPSPSPSTSTSPNSDIGLGFLHHHSSGGGD
GVPLPRAGGGALNLLLQSTKFKEMLERTSAVDSPSTTPEADNPPRCSFPD
DIQTYFECEDPGVGDDVIFGDLNSFAAPLIEGEWD

A nucleotide sequence that encodes the Persea americana WRI3 protein with SEQ ID NO: 4 is shown below as SEQ ID NO: 5:

ATGGGGAAAT CATCAAAACC CAACAACAAC AGCAGTGACA
ACAATAGCAA GTCGACTACA AAGGTGAAAA GAACGAGAAA
AAGCGTGCCA AGAGAATCAC CCACACAACG TAGCTCCATC
TACAGAGGTG TCACAAGGCA TCGATGGACA GGGAGATATG
AAGCTCATTT GTGGGATAAA AATTGCTGGA ATGAGACCCA
AAACAAGAAA GGAAGACAAG GTGCTTATGA TGATGAAACT
GCAGCTGCAC ACGCATATGA CTTGGCAGCA CTGAAGTACT
GGGGACAGGA CACTATCCTC AACTTTCCTG CATCTACATA
TGAGGAAGAA CTGAAAGAAA TGGAAGGACA ATCAAAAGAA
GAATACATTG GATCCTTGAG AAGGAAAAGT AGTGGCTTTT
CCAGGGGTGT ATCAAAGTAC AGAGGTGTTG CAAGGCATCA
TCACAATGGA AGATGGGAAG CTCGCATTGG AAGAGTGTTT
GGGAACAAAT ACCTTTATCT GGGAACCTAT GCCACCCAAG
AAGAAGCAGC CACAGCTTAC GACATGGCGG CAATTGAGTA
CAGGGGACTT AACGCCGTTA CGAATTTCGA TCTGAGCCGT
TACATCAAAT GGCTTCGCCC CGGCTCACAA AACCCTAATT
CCAACACTGA CCAAAACCCT AACCCTAGCC CTAGCCCTAG
CACTAGCACT AGCCCAAATT CTGATATAGG GCTGGGCTTC
CTCCACCACC ATAGCTCAGG CGGCGGAGAC GGGGTCCCTC
TGCCCCGTGC CGGGGGCGGC GCCCTCAACC TACTGCTGCA
GTCGACGAAA TTCAAGGAGA TGTTGGAGAG GACGTCGGCG
GTGGAC TCCC CGTCGACGAC GCCGGAAGCA GACAACCCGC
CGCGGTGCAG CTTTCCCGAC GACATACAGA CGTACTTTGA
GTGCGAGGAT CCAGGCGTGG GGGATGACGT CATCTTCGGC
GATCTGAACT CGTTCGCAGC GCCTTTGATC GAGGGCGAGT
GGGACTAG

A WRI3 protein sequence from Sorghum is shown below as SEQ ID NO: 6.

MAKPRKNSAAANNNNSSSNGAGDLTPRAKPKRTRKSVPRESPTQRSSVY
RGVTRHRWTGRFEAHLWDKNSWNESQNKKGKQVYLGAYDDEEAAARAYD
LAALKYWGPDTILNFPASAYEGEMKGMEGQSREEYIGSLRRKSSGFSRG
VSKYRGVARHHHNGRWEARIGRVFGNKYLYLGTYATQEEAAMAYDMAAI
EYRGLNAVTNFDLSRYIKWLRPGAGGMAAAAAAAQNPHPMLGGLAQQLL
LPPPADTTTTDGAGAAAFQHDHHGAEAFPLPPRTSLGHTPTTSALSLLL
QSPKFKEMIQRTESGTTTTTTTTSSLSSSPPPTPSPSPPRRSPAPTQPP
VQAAARDASPHQRGFPEDVQTFFGCEDTAGIDVEALFFGDLAAYATPAF
HFEMDL

A nucleotide sequence that encodes the Sorghum WRI3 protein with SEQ ID NO: 6 is shown below as SEQ ID NO: 7:

ATGGCGAAGC CTCGCAAGAA CAGCGCCGCC
GCCAACAACA ACAACAGCAG CAGCAACGGC
GCCGGCGACC TGACGCCGCG CGCGAAGCCG
AAGCGCACGC GGAAGAGCGT GCCCCGGGAG
TCCCCCACGC AGCGCAGCTC CGTCTACCGC
GGCGTCACAC GGCACCGGTG GACGGGGAGG
TTCGAGGCTC ACCTGTGGGA CAAGAACAGC
TGGAACGAGT CCCAGAACAA GAAGGGCAAG
CAAGTTTACC TCGGCGCGTA CGACGACGAG
GAGGCAGCGG CGCGGGCGTA TGACTTGGCG
GCATTGAAGT ACTGGGGCCC CGACACCATC
CTCAACTTCC CGGCGTCTGC ATATGAAGGA
GAGATGAAAG GAATGGAGGG CCAGTCCAGG
GAAGAGTATA TTGGATCCTT GAGGAGGAAA
AGCAGTGGGT TCTCCAGAGG TGTCTCCAAA
TACCGAGGTG TCGCGAGACA TCACCACAAC
GGGAGATGGG AGGCGAGGAT CGGTCGTGTT
TTCGGAAACA AGTATCTCTA CCTCGGGACT
TACGCGACGC AGGAGGAGGC GGCGATGGCG
TACGACATGG CGGCGATCGA GTACCGTGGC
CTGAACGCCG TCACAAACTT CGACCTCAGC
CGCTACATCA AGTGGCTCCG CCCGGGCGCC
GGCGGGATGG CAGCCGCGGC GGCGGCGGCG
CAGAACCCGC ACCCGATGCT GGGCGGCCTG
GCGCAGCAGC TGCTGCTGCC GCCGCCGGCG
GACACCACCA CCACCGACGG CGCCGGCGCC
GCGGCGTTCC AGCACGACCA CCACGGCGCC
GAGGCGTTCC CGCTGCCGCC CAGGACGTCG
CTGGGCCACA CGCCCACGAC GTCGGCGCTC
AGCCTGCTGC TGCAGTCGCC CAAGTTCAAG
GAGATGATCC AGCGGACCGA GAGCGGCACC
ACCACCACTA CCACCACGAC ATCGTCGTTG
TCCTCGTCGC CGCCGCCCAC GCCGTCCCCG
TCCCCGCCGC GGCGGTCGCC AGCGCCGACG
CAGCCGCCGG TGCAGGCGGC GGCCAGGGAC
GCCTCGCCGC ACCAGCGCGG CTTCCCCGAG
GACGTACAGA CATTCTTCGG CTGCGAGGAC
ACCGCGGGCA TCGACGTGGA AGCCCTCTTC
TTTGGCGACC TCGCCGCGTA CGCGACGCCG
GCGTTCCACT TCGAGATGGA CTTGTAG

A AtWRI3 protein sequence from Arabidopsis thaliana is shown below as SEQ ID NO: 8.

| | |
|---|---|
| 1 | MFIAVEVSPV MEDITRQSKK TSVENETGDD QSATSVVLKA KRKRRSQPRD |
| 51 | APPQRSSVHR GVTRHRWTGR YEAHLWDKNS WNETQTKKGR QVYLGAYDEE |
| 101 | DAAARAYDLA ALKYWGRDTI LNFPLCNYEE DIKEMESQSK EEYIGSLRRK |
| 151 | SSGFSRGVSK YRGVAKHHHN GRWEARIGRV FGNKYLYLGT YATQEEAAIA |
| 201 | YDIAAIEYRG LNAVTNFDIS RYLKLPVPEN PIDTANNLLE SPHSDLSPFI |
| 251 | KPNHESDLSQ SQSSSEDNDD RKTKLLKSSP LVAEEVIGPS TPPEIAPPRR |
| 301 | SFPEDIQTYF GCQNSGKLTA EEDDVIFGDL DSFLTPDFYS ELNDC |

A nucleotide sequence that encodes the AtWRI3 protein with SEQ. ID. NO. 8 is shown below as SEQ ID NO: 9:

| | |
|---|---|
| 1 | CTAAATGGTT TGATTACGAT TAGGAATTTT AATAAATGAG AAATCTAATT |
| 51 | ACAGAAGTAC AAAGAGGGCC GTAGCAAAGG AAATCTTTTA AAGCAATAGC |
| 101 | AATTAAAAAG GCTAAAAAAA ATAAAACGGA AAATTATAAT TTTAAATACT |
| 151 | CAACCATTTC TCTTTATAGT TTCCTTATAA AACAAAAACA AAAACCATTC |
| 201 | CATCAATTTC TCATCCTTGT TGTTTTCTTC TTCTTCTTCT TCCTCTTCTT |
| 251 | TTATGTTCAT CGCCGTCGAA GTTTCTCCGG TAATGGAAGA CATCACACGG |
| 301 | CAGAGCAAAA AAACTTCGGT TGAGAATGAA ACCGGCGATG ATCAGTCAGC |
| 351 | AACATCAGTA GTCCTTAAAG CTAAACGCAA ACGCCGATCG CAACCACGAG |
| 401 | ACGCTCCACC CCAACGTAGC TCCGTCCATA GAGGCGTCAC AAGGCATCGA |
| 451 | TGGACTGGAA GGTACGAAGC ACATTTGTGG GATAAGAATA GTTGGAACGA |
| 501 | AACTCAGACC AAGAAAGGAA GACAAGTATA TTTAGGGGCA TATGACGAGG |
| 551 | AAGATGCAGC AGCACGTGCC TACGACTTAG CAGCATTGAA ATATTGGGGA |
| 601 | CGAGACACCA TCTTGAACTT CCCTTTGTGT AATTATGAAG AAGACATCAA |
| 651 | AGAAATGGAA AGCCAGTCAA AGGAAGAGTA TATTGGATCT TTGAGAAGAA |
| 701 | AAAGTAGTGG GTTTTCACGA GGTGTATCAA AATACAGAGG CGTTGCAAAG |
| 751 | CATCACCACA ATGGGAGATG GGAAGCTCGA ATCGGAAGAG TGTTTGGCAA |
| 801 | TAAATATTTA TACCTTGGAA CTTACGCGAC GCAAGAAGAA GCAGCTATAG |
| 851 | CGTACGATAT CGCAGCTATC GAGTACCGTG GACTCAACGC CGTTACTAAC |
| 901 | TTCGACATCA GCCGTTATCT GAAACTCCCG GTGCCGGAGA ACCCTATCGA |
| 951 | TACCGCGAAT AATCTCCTCG AGAGTCCGCA TTCTGATCTT AGCCCATTTA |
| 1001 | TAAAACCTAA CCACGAGTCT GACTTATCAC AGAGTCAATC TTCGTCAGAG |
| 1051 | GACAACGATG ATCGGAAAAC AAAGCTCTTG AAGTCGTCAC CTTTAGTGGC |
| 1101 | AGAGGAGGTA ATCGGACCAT CGACGCCACC TGAGATTGCT CCGCCTCGTC |
| 1151 | GGAGCTTCCC GGAAGATATC CAGACGTATT TCGGGTGTCA AAACTCCGGC |
| 1201 | AAGTTAACGG CGGAGGAAGA TGATGTTATC TTCGGTGATT TAGATTCTTT |
| 1251 | CCTTACGCCT GATTTCTACA GCGAGTTAAA TGATTGCTAA AGTGTTGTTC |
| 1301 | TTCTGATAAG TTTTGTTTTT TAGTTGTTCA GAATCTCGGT TGTGAAAATC |
| 1351 | AACATTGACA CATCGATTAT TCTTTCTTGT GACAATCTTA TATAATAAAG |
| 1401 | TTTGAATCTT TTTAAC |

Methods described herein include methods of growing a plant seed or plant comprising introducing into at least one plant cell at least one transgene or expression cassette encoding a polypeptide for SpWRI3 to generate one or more transformed plant cells; and generating a plant from the one or more transformed plant cell(s), wherein the plant seed or plant is tolerant to heavy metal stress, salt stress, or a combination thereof.

Methods of growing the plant seed or plant from the transformed plant cell(s) can include contacting the plant seed or plant with flue gas desulfurization (FGD) wastewater while generating the plant from one or more transformed plant cell(s). FGD wastewater is water from coal-fired power plants used to remove sulfur dioxide (SO₂) air emissions. FGD wastewater is often saturated with calcium sulfate and can contain metals and chlorides. Table 1 shows the metals found in the FGD wastewater used in the Examples below. Table 2 shows the pH of FGD wastewater and tap water used in the Examples below.

TABLE 1
Major metal analysis in FGD wastewater based on the published
literature (collected before the chemical treatment):

| | Concentration | |
|---|---|---|
| Metal | (mg/L) | Method |
| Aluminum | 0.248 | EPA200.7Rev4.4-1994 |
| Arsenic | <0.0050 | EPA200.7Rev4.4-1994 |
| Boron | 268 | EPA200.7Rev4.4-1994 |
| Cadmium | 0.046 | EPA200.7Rev4.4-1994 |
| Calcium | 865 | EPA200.7Rev4.4-1994 |
| Cobalt | 0.857 | EPA200.7Rev4.4-1994 |
| Copper | 0.016 | EPA200.7Rev4.4-1994 |
| Iron | 0.14 | EPA200.7Rev4.4-1994 |
| Lead | <0.005 | EPA200.7Rev4.4-1994 |
| Magnesium | 3320 | EPA200.7Rev4.4-1994 |
| Manganese | 39.3 | EPA200.7Rev4.4-1994 |
| Nickel | 0.463 | EPA200.7Rev4.4-1994 |
| Potassium | 51.5 | EPA200.7Rev4.4-1994 |
| Sodium | 120 | EPA200.7Rev4.4-1994 |
| Strontium | 9.31 | EPA200.7Rev4.4-1994 |
| Selenium | 0.1670 | SM3114C-1997ATP |
| Nitrate as N | 14.3 | EPA300.0Rev2.1-1993 |
| Zinc | 0.353 | EPA200.7Rev4.4-1994 |

TABLE 2
The pH in FGD wastewater (collected before
the chemical treatment) and tap water:

| | |
|---|---|
| FGD wastewater | 9.5 |
| Tap water | 7.0 |

Methods of growing the plant seed or plant from the transformed plant cell(s) can include contacting the plant seed or plant with a marginal soil while generating the plant from one or more transformed plant cell(s). For example, the marginal soil can be reclaimed coal mine soil. Growth of the transformed plant cell(s) expressing the SpWRI3 gene can provide a method of phytoremediation of the marginal soil.

Methods of growing the plant seed or plant from the transformed plant cell(s) can include generating the plant seed or plant in salt stress conditions. Salt stress conditions can include growing the plant seed or plant in a medium that includes, for example, 50 mM, 75 mM, 100 mM, 120 mM, 150 mM, or 200 mM NaCl or seawater or sodic soils or industrial contaminant soil rich in gypsum. Also, saline water or brine from industrial or food processing waste such as tanning leather, manufacturing textile, or certain types of food. This brine is often consisting high levels of salt contamination. The transformed plant cell(s) can generate the plant seed or plant that is tolerant to highly saline soils. Saline soils can include sodium salt and can become sodic soil. Saline soils can also contain calcium, magnesium, potassium, sulfate, chloride, carbonate, and bicarbonate. According to U.S. Salinity Laboratory Staff (1954), saline soil has an electrical conductivity (EC) of the saturated paste extract of more than 4 dS/m, a value that corresponds to approximately 40 mmol salts per liter.

The nucleic acids and polypeptides allow the identification and isolation of related nucleic acids and their encoded proteins that provide for the production of healthy plants with modulated lipid content, such as increased lipid content.

Plant cells, plant seeds, and plants disclosed herein can comprise an expression cassette comprising a promoter operably linked to an original or codon-optimized nucleic acid segment encoding a polypeptide for SpWRI3. The polypeptide for SpWRI3 has at least about 95% (such as at least 95%, 96%, 97%, 98%, or 99%) sequence identity to SEQ ID NO: 1.

The SpWRI3 nucleic acids described herein can include any nucleic acid that can selectively hybridize to a nucleic acid having the nucleotide sequence of any of SEQ ID NOs: 2 or 4 under stringent conditions. Desirably, the nucleic acid that can selectively hybridize to a nucleic acid described herein encodes a polypeptide having activity characteristic of the polypeptide encoded by the nucleic acid to which it hybridizes. The activity can be the same or modulated, such as increased.

The term “selectively hybridize” includes hybridization, under stringent hybridization conditions, of a nucleic acid sequence to a specified nucleic acid target sequence (e.g., any of the SEQ ID NO: 2 or 4 nucleic acids) to a detectably greater degree (e.g., at least 2-fold over background) than its hybridization to non-target nucleic acid sequences. Such selective hybridization substantially excludes non-target nucleic acids. Selectively hybridizing sequences typically have about at least about 60% (such as 60%) sequence identity, at least about 70% (such as 70%) sequence identity, at least about 75% (such as 75%), at least about 80% (such as 80%), at least about 85% (such as 85%), at least about 90% (such as 90%), at least about 95% (such as 95%), at least about 96% (such as 96%), at least about 97% (such as 97%), at least about 98% (such as 98%), at least about 99% (such as 99%), at least about 60% to at least about 99% (such as about 60% to 99%, 60% to about 99%, or 60%-99%) sequence identity, at least about 70% to at least about 99% (such as about 70% to 99%, 70% to about 99%, or 70%-99%) sequence identity, at least about 80% to at least about 99% (such as about 80% to 99%, 80% to about 99%, or 80%-99%) sequence identity, at least about 90% to at least about 99% (such as about 90% to 99%, 90% to about 99%, or 90%-99%), at least about 90% to about 95% (such as about 90% to 95%, 90% to about 95%, or 90%-95%) sequence identity, at least about 90% to about 95% (such as about 90% to 99%, 90% to about 99%, or 90%-99%) sequence identity, at least about 95% to about 97% (such as about 95% to 97%, 95% to about 97% or 95%-97%) sequence identity, at least about 97% to about 99% (such as about 97% to 99%, 97% to about 99% or 97%-99%) sequence identity, or 100% sequence identity (or complementarity) with each other. In some embodiments, a selectively hybridizing sequence has at least about 80% (such as 80%, 85%, 90%, 92.5%, 95%, 97.5%, 98%, or 99%) sequence identity or complementarity with SEQ ID NO: 2 or 4.

Thus, the nucleic acids include those with about 500 of the same nucleotides as SEQ ID NO: 2 or 4, or about 600 of the same nucleotides, or about 700 of the same nucleotides, or about 800 of the same nucleotides, or about 900 of the same nucleotides, or about 1000 of the same nucleotides, or about 1100 of the same nucleotides, or about 1200 of the same nucleotides as SEQ ID NO: 2 or 4. The identical nucleotides can be distributed throughout the nucleic acid or the protein, and need not be contiguous.

Note that if a value of a variable that is necessarily an integer, e.g., the number of nucleotides or amino acids in a nucleic acid or protein, respectively, is described as a range, e.g., 90-99% sequence identity, what is meant is that the value can be any integer between 90 and 99 inclusive, i.e., 90, 91, 92, 93, 94, 95, 96, 97, 98 or 99, or any range between 90 and 99 inclusive, e.g., 91-99%, 91-98%, 92-99%, etc.

### Promoters:

The SpWRI3 nucleic acids can be operably linked to a promoter, which provides for the expression of mRNA from the SpWRI3 nucleic acids. The promoter is typically a promoter functional in plants and/or seeds and can be a promoter functional during plant growth and development. A SpWRI3 nucleic acid is operably linked to the promoter when it is located downstream from the promoter, thereby forming an expression cassette.

Most endogenous genes have regions of DNA that are known as promoters, which regulate gene expression. Promoter regions are typically found in the flanking DNA upstream from the coding sequence in both prokaryotic and eukaryotic cells. A promoter sequence provides for the regulation of transcription of the downstream gene sequence and typically includes about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, constitutive or inducible. A strong promoter provides for a high level of gene expression, whereas a weak promoter provides for a very low level of gene expression. An inducible promoter is a promoter that allows gene expression to be turned on and off in response to an exogenously added agent, or to an environmental or developmental stimulus. For example, a bacterial promoter such as the Ptac promoter can be induced to varying levels of gene expression depending on the level of isothiopropylgalactoside added to the transformed cells. Promoters can also provide for tissue-specific or developmental regulation. An isolated promoter sequence that is a strong promoter for heterologous DNAs is advantageous because it provides a sufficient level of gene expression for easy detection and selection of transformed cells and provides for a high level of gene expression when desired. A constitutive promoter is a promoter that is unregulated and allows for continual transcription of a gene.

Expression cassettes generally include, but are not limited to, a plant promoter such as the CaMV 35S promoter (Odell et al., Nature. 313:810 812 (1985)), or others such as CaMV 19S (Lawton et al., Plant Molecular Biology. 9:315 324 (1987)), nos (Ebert et al., Proc. Natl. Acad. Sci. USA. 84:5745 5749 (1987)), Adh1 (Walker et al., Proc. Natl. Acad. Sci. USA. 84:6624 6628 (1987)), sucrose synthase (Yang et al., Proc. Natl. Acad. Sci. USA. 87:4144 4148 (1990)), α-tubulin, ubiquitin (e.g. Arabidopsis Ubiquitin 10 promoter), actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579 589 (1989)) or those associated with the R gene complex (Chandler et al., The Plant Cell. 1:1175 1183 (1989)). Further, suitable promoters include the poplar xylem-specific secondary cell wall-specific cellulose synthase 8 promoter, cauliflower mosaic virus promoter, the Z10 promoter from a gene encoding a 10 kDa zein protein, a Z27 promoter from a gene encoding a 27 kDa zein protein, inducible promoters, such as the light-inducible promoter derived from the pea rbcS gene (Coruzzi et al., EMBO J. 3:1671 (1971)) and the actin promoter from rice (McElroy et al., The Plant Cell. 2:163 171 (1990)). Seed-specific promoters, such as the phaseolin promoter from beans, may also be used (Sengupta Gopalan, Proc. Natl. Acad. Sci. USA. 83:3320 3324 (1985)). A strong constitutive promoter such as Arabidopsis ubiquitin-10 gene promoter (Grefen et al., The Plant Journal., 64(2), 355-365 (2010).

### Transformation of Plant Cells

Plant cells can be transformed to include one or more WRI3 transcription factor transgenes (e.g., SpWRI3), for example, by a transformation of the plant cells with an expression cassette or expression vector using Agrobacterium-mediated or gene gun or flower dip. The frequency of occurrence of cells taking up exogenous (foreign) DNA can sometimes be low. However, certain cells from virtually any dicot or monocot species can be stably transformed, and these cells can be regenerated into transgenic plants, through the application of the techniques disclosed herein. The plant cells, plants, and seeds can therefore be monocotyledons or dicotyledons.

The cell(s) that undergo transformation may be in a suspension cell culture or maybe in an intact plant part, such as an immature embryo, or in specialized plant tissue, such as callus, such as a Type I or Type II callus.

Transformation of the cells of the plant tissue source can be conducted by any one of a number of methods available to those of skill in the art. Examples include: Transformation by direct DNA transfer into plant cells by electroporation (U.S. Pat. Nos. 5,384,253 and 5,472,869, Dekeyser et al., The Plant Cell. 2:591 602 (1990)); direct DNA transfer to plant cells by PEG precipitation (Hayashimoto et al., Plant Physiol. 93:857 863 (1990)); direct DNA transfer to plant cells by microprojectile bombardment (McCabe et al., Bio/Technology. 6:923 926 (1988); Gordon Kamm et al., The Plant Cell. 2:603 618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli derived plasmid cloning vector. In the case of viral vectors, it is desirable that the system retain replication functions, but lack functions for disease induction.

One method for dicot transformation, for example, involves infection of plant cells with Agrobacterium tumefaciens using the leaf disk protocol (Horsch et al., Science 227:1229 1231 (1985). Some dicots are also transformed using flower dip such as Arabidopsis, camelina, pennycress, and other plants (Bent, 2006). Monocots such as Zea mays can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature Zea mays embryos can be transformed by accelerated particle treatment as described by Gordon Kamm et al. (The Plant Cell. 2:603 618 (1990)) or U.S. Pat. Nos. 5,489,520; 5,538,877 and 5,538,880, cited above. Excised immature embryos can also be used as the target for transformation prior to tissue culture induction, selection, and regeneration as described in U.S. application Ser. No. 08/112,245 and PCT publication WO 95/06128. Furthermore, methods for the transformation of monocotyledonous plants utilizing Agrobacterium tumefaciens have been described by Hiei et al. (European Patent 0 604 662, 1994) and Saito et al. (European Patent 0 672 752, 1995).

Methods such as microprojectile bombardment or electroporation can be carried out with “naked” DNA where the expression cassette may be simply carried, for example, on any E. coli-derived plasmid cloning vector. In the case of viral vectors, it is desirable that the system retain replication functions, but lack functions for disease induction.

The choice of plant tissue source for transformation will depend on the nature of the host plant and the transformation protocol. Useful tissue sources include callus, suspension culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells. Type I or Type II embryonic maize callus and immature embryos are exemplary Zea mays tissue sources. The selection of tissue sources for the transformation of monocots is described in detail in U.S. application Ser. No. 08/112,245 and PCT publication WO 95/06128.

The transformation is carried out under conditions directed to the plant tissue of choice. The plant cells or tissue are exposed to the DNA or RNA carrying the targeting vector and/or other nucleic acids for an effective period of time. This may range from a less than one-second pulse of electricity for electroporation to a 2-3 day co-cultivation in the presence of plasmid-bearing Agrobacterium cells. Buffers and media used will also vary with the plant tissue source and transformation protocol. Many transformation protocols employ a feeder layer of suspended culture cells (tobacco or Black Mexican Sweet corn, for example) on the surface of solid media plates, separated by a sterile filter paper disk from the plant cells or tissues being transformed.

Where one wishes to introduce DNA by means of electroporation, it is contemplated that the method of Krzyzek et al. (U.S. Pat. No. 5,384,253) may be advantageous. In this method, certain cell wall degrading enzymes, such as pectin degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic cells were bombarded with intact cells of the bacteria E. coli or Agrobacterium tumefaciens containing plasmids with either the β-glucouronidase or bar gene engineered for expression in maize. Bacteria were inactivated by ethanol dehydration prior to bombardment. A low level of transient expression of the β-glucouronidase gene was observed 24-48 hours following DNA delivery. In addition, stable transformants containing the bar gene can be recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, is that the isolation of protoplasts (Christou et al., PNAS. 84:3962 3966 (1987)), the formation of partially degraded cells, or the susceptibility to Agrobacterium infection is not required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon Kamm et al., The Plant Cell. 2:603 618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing damage inflicted on the recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the macroprojectile stopping plate. If desired, one or more screens are also positioned between the acceleration device and the cells to be bombarded. Through the use of techniques set forth here in one may obtain up to 1000 or more foci of cells transiently expressing a marker gene. The number of cells in a focus which express the exogenous gene product 48 hours post bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macroprojectiles or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells, and which may therefore influence transformation and integration efficiencies. For example, the osmotic state, tissue hydration and the subculture stage or cell cycle of the recipient cells may be adjusted for optimum transformation. Execution of such routine adjustments will be known to those of skill in the art.

Examples of plants and/or plant cells that can be modified as described herein include alfalfa (e.g., forage legume alfalfa), algae, avocado, barley, broccoli, Brussels sprouts, cabbage, canola, cassava, cauliflower, cole vegetables, collards, corn, crucifers, duckweed, grain legumes, grasses (e.g., forage grasses), jatropa, kale, kohlrabi, maize, miscanthus, mustards, nut sedge, oats, oil firewood trees, oilseeds, potato, radish, rape, rapeseed, rice, rutabaga, sorghum, soybean, sugar beets, sugarcane, sunflower, switchgrass, tobacco, tomato, turnips, camelina, pennycress, winter rye, sweet potato, poplar tree, rice and wheat. In some embodiments, the plant is a Brassicaceae or other Solanaceae species. In some embodiments, the plant or cell can be a maize plant or cell. In some embodiments, the plant is not a species of Arabidopsis, for example, in some embodiments, the plant is not Arabidopsis thaliana. In some embodiments, the plant can be a species of Arabidopsis, such as Arabidopsis thaliana.

An exemplary embodiment of methods for identifying transformed cells involves exposing the bombarded cultures to a selective agent, such as a metabolic inhibitor, an antibiotic, herbicide, or the like. Cells that have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on a nonselective medium and subsequently transferred to a medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for the selection of transformants, but the technique of this invention is not limited to them.

An example of a screenable marker trait is the red pigment produced under the control of the R-locus in maize. This pigment may be detected by culturing cells on a solid support containing nutrient media capable of supporting growth at this stage and selecting cells from colonies (visible aggregates of cells) that are pigmented. These cells may be cultured further, either in suspension or on solid media. The R-locus is useful for the selection of transformants from bombarded immature embryos. In a similar fashion, the introduction of the C1 and B genes will result in pigmented cells and/or tissues.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light-sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for the identification of transformed cells. For example, selection with a growth-inhibiting compound, such as bialaphos or glyphosate at concentrations below those that cause 100% inhibition followed by a screening of growing tissue for expression of a screenable marker gene such as luciferase would allow one to recover transformants from cell or tissue types that are not amenable to selection alone. In an illustrative embodiment, embryogenic Type II callus of Zea mays L. can be selected with sub-lethal levels of bialaphos. Slowly growing tissue was subsequently screened for expression of the luciferase gene and transformants can be identified.

### Regeneration and Seed Production

Cells that survive the exposure to the selective agent, or cells that have been scored positive in a screening assay, are cultured in media that supports the regeneration of plants. One example of a growth regulator that can be used for such purposes is dicamba or 2,4-D. However, other growth regulators may be employed, including NAA, NAA+2,4-D or picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO2, and at about 25-250 microeinsteins/sec·m2 of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to have the mutations. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants in order to introgress the mutations into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced transcription factors (TFs), e.g., SpWRI3, or expression cassette, the plant is self-pollinated at least once in order to produce a homozygous backcross converted inbred containing the mutations. The progeny of these plants are true breeding.

Alternatively, seed from transformed mutant plant lines regenerated from transformed tissue cultures is grown in the field and self-pollinated to generate true breeding plants.

Seed from the fertile transgenic plants can then be evaluated for the presence of the desired TFs, the expression cassette, and/or the expression of the desired mutant protein. Transgenic plant and/or seed tissue can be analyzed using standard methods such as SDS polyacrylamide gel electrophoresis, liquid chromatography (e.g., HPLC) or other means of detecting a mutation.

Once a transgenic plant with a mutant sequence and having improved growth and insect resistance is identified, seeds from such plants can be used to develop true breeding plants. The true breeding plants are used to develop a line of plants with an increase insect resistance relative to wild type, and acceptable growth characteristics while still maintaining other desirable functional agronomic traits. Adding the mutation to other plants can be accomplished by backcrossing with this trait and with plants that do not exhibit this trait and studying the pattern of inheritance in segregating generations. Those plants expressing the target trait (insect resistance, good growth) in a dominant fashion are preferably selected. Backcrossing is carried out by crossing the original fertile transgenic plants with a plant from an inbred line exhibiting desirable functional agronomic characteristics while not necessarily expressing the trait of an increased insect resistance and good plant growth. The resulting progeny are then crossed back to the parent that expresses the increased insect resistance and good plant growth. The progeny from this cross will also segregate so that some of the progeny carry the trait and some do not. This backcrossing is repeated until an inbred line with the desirable functional agronomic traits, and with expression of the trait involving an increase in insect resistance and good plant growth. Such insect resistance and good plant growth can be expressed in a dominant fashion.

The new transgenic plants can also be evaluated for a battery of functional agronomic characteristics such as growth, lodging, kernel hardness, yield, resistance to disease and insect pests, drought resistance, and/or herbicide resistance.

Plants that may be improved by these methods include but are not limited to agricultural plants of all types, oil and/or starch plants (canola, potatoes, lupins, sunflower, and cottonseed), forage plants (alfalfa, clover, and fescue), grains (maize, wheat, barley, oats, rice, sorghum, millet and rye), grasses (switchgrass, prairie grass, wheat grass, Sudan grass, sorghum, straw-producing plants), softwood, hardwood and other woody plants (e.g., those used for paper products such as poplar species, pine species, and eucalyptus). In some embodiments, the plant is a gymnosperm. Examples of plants useful for pulp and paper products include most pine species such as loblolly pine, Jack pine, Southern pine, Radiata pine, spruce, Douglas fir, and others. Hardwoods that can be modified as described herein include aspen, poplar, eucalyptus, and others. Plants useful for making biofuels and ethanol include corn, grasses (e.g., miscanthus, switchgrass, and the like), as well as trees such as poplar, aspen, willow, and the like. Plants useful for generating dairy forage include legumes such as alfalfa, as well as forage grasses such as bromegrass, and bluestem.

### Determination of Stably Transformed Plant Tissues

To confirm the presence of TFs, or expression cassette in the regenerating plants, or seeds or progeny derived from the regenerated plant, a variety of assays may be performed. Such assays include, for example, molecular biological assays available to those of skill in the art, such as Southern and Northern blotting and PCR; biochemical assays, such as detecting the presence of a protein product, e.g., by immunological means (ELISAs and Western blots) or by enzymatic function; plant part assays, such as leaf, seed or root assays; and also, by analyzing the phenotype of the whole regenerated plant.

Whereas DNA analysis techniques may be conducted using DNA isolated from any part of a plant, RNA may only be expressed in particular cells or tissue types and so RNA for analysis can be obtained from those tissues. PCR techniques may also be used for the detection and quantification of RNA produced from introduced TFs or of RNA expressed from an introduced expression cassette. For example, PCR also be used to reverse transcribe RNA into DNA, using enzymes such as reverse transcriptase, and then this DNA can be amplified through the use of conventional PCR techniques.

For example, if no amplification of TFs mRNAs is observed, then a deletion mutation has successfully been introduced.

Information about mutations can also be obtained by primer extension or single nucleotide polymorphism (SNP) analysis.

Further information about the nature of the RNA product may be obtained by Northern blotting. This technique will demonstrate the presence of an RNA species and give information about the integrity of that RNA. The presence of some mutations can be detected by Northern blotting. The presence or absence of an RNA species (e.g., TF RNA) can also be determined using dot or slot blot Northern hybridizations. These techniques are modifications of Northern blotting and also demonstrate the presence or absence of an RNA species.

While Southern blotting and PCR may be used to detect the presence of TFs or the presence of the expression cassette, they do not provide information as to whether the preselected DNA segment is being expressed. Expression may be evaluated by specifically identifying the protein products of the introduced expression cassette or evaluating the phenotypic changes brought about by such mutation.

Assays for the production and identification of specific proteins may make use of physical chemical, structural, functional, or other properties of the proteins. Unique physical chemical or structural properties allow the proteins to be separated and identified by electrophoretic procedures, such as native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products, or the absence thereof, that have been separated by electrophoretic techniques. Additional techniques may be employed to confirm the identity of a mutation such as evaluation by screening for reduced transcription (or no transcription) of TF by screening for TF expression, or by amino acid sequencing following purification. The Examples of this application also provide assay procedures for detecting the TFs or evaluating the seed oil in the resulting plants. Other procedures may be additionally used.

The following Examples illustrate some of the experimental work involved in the development of the invention.

### Example 1

### Introduction

Plants depend on certain heavy metals as micronutrients, which are required as cofactors for biochemical reactions, but heavy metals become toxic in excess (1, 2). Indeed, crops increasingly experience heavy-metal stress due to anthropogenic activities such as the excessive use of pesticides and fertilizer in agriculture, improper treatment of industrial waste, and intensive mining. These activities compromise the quality of soil and freshwater resources and reduce agricultural production worldwide (3, 4). In addition, heavy metals are readily soluble in water and easily taken up by plants, resulting in their entry into the food web. The biomagnified levels of heavy metals can then have adverse health impacts in humans (5, 6). High levels of heavy metals in agricultural products thus have negative effects on the downstream food industry and supply chain.

Membrane lipids are major targets of environmental stress, leading to changes in their biosynthesis and composition (7, 8). In plants, the chloroplast stroma is the primary site of fatty acid biosynthesis for the production of membrane lipids such as monogalactosyldiacylglycerol (MGDG), digalactosyldiacylglycerol (DGDG), phosphatidylglycerol (PG), and sulfoquinovosyl diacylglycerol (SQDG), the main structural components of photosynthetic membranes. MGDG and DGDG represent 70-80% of chloroplast lipids (9, 7, 10). Considerable progress has been made toward understanding membrane lipid modifications in heavy-metal-stressed plants (11-13). For example, heavy-metal treatment altered lipid biosynthesis, lipid composition, and fatty acid desaturation in tomato (Solanum lycopersicum) (14), maize (Zea mays) (15), the freshwater vascular plant Hydrilla verticillata (16), and Arabidopsis (Arabidopsis thaliana) (17-19). In addition, heavy-metal exposure inhibited photosynthetic pigment development and decreased crop yield in rice (Oryza sativa) (20).

The damage to cellular membranes caused by heavy-metal exposure can cause electrolyte leakage and excessive accumulation of reactive oxygen species (ROS). The resulting oxidative stress increases lipid peroxidation and malondialdehyde (MDA) levels and leads to the inactivation of enzymes and DNA damage (21). ROS can damage cellular membranes, resulting in the production of free fatty acids (FFAs), fatty acid esters, phosphatidic acid (PA), phosphatidylinositol (PI), sphingolipids, oxylipins, N-acylethanolamines, and others (1, 22). ROS are toxic to cells, and their accumulation can disturb cellular signaling pathways, induce oxidative stress, and lead to cell death or lipotoxicity (23-25). Plants have evolved sophisticated antioxidant defense systems against heavy-metal stress, such as the production of ascorbic acid, proline, glutathione (GSH), phenolic compounds, and flavonoids to scavenge ROS (21, 26). Additionally, plants sequester toxic lipid intermediates such as free fatty acids (FFAs) from the damaged membrane in triacylglycerols (TAGs) to prevent cellular toxicity (27-30).

Attention has been given to aquatic plants for phytoremediation of heavy metals due to their aquatic habit and their ability to take up heavy metals through their roots and leaves via active and passive transport (31, 32). In contrast to terrestrial plants, aquatic plants are completely exposed to polluted water, and many of them accumulate heavy metals. Although excess metals interfere with plant metabolic processes and inhibit growth, fertility, and yield, ultimately resulting in plant death, several aquatic plants have an innate tolerance of heavy metals in micro quantities (33, 34). In Spirodela polyrhiza, excess copper (Cu) and iron (Fe) result in necrosis, reduced chlorophyll and protein biosynthesis, and reduced nutrient uptake (35). In Azolla caroliniana and Lemna minor, high concentrations of As and Cu negatively affect biomass production and chlorophyll content (36). Similarly, an overabundance of Cu, Pb, Cd, or their combination reduces biomass and chlorophyll content in Vallisneria natans (37). High concentrations of Pb and Zn adversely affect chlorophyll and soluble protein contents in L. minor (26). Some progress has been made in understanding the responses of these plants to heavy metal stress. In S. polyrhiza, glutathione (GSH) content increases with increasing Cu and Fe concentrations, and a decrease in proline contents is observed in response to increased concentrations of heavy metals (35). Similarly, exposure of L. minor to Cu leads to changes in the concentrations of ascorbate and GSH (38).

Heavy-metal resistance mechanisms mediated by membrane lipid modification in aquatic plants remain to be revealed. S. polyrhiza, a member of the duckweed family, is among the smallest and simplest monocotyledonous flowering plants; they float in nutrient-rich aquatic environments worldwide (39). Its aquatic habit, fast growth, high biomass, and small genome size (158 Mb)—the smallest in the duckweed family—make S. polyrhiza amenable to basic and applied research (40, 41). Due to its robust growth habitat, physiology, and genetics, S. polyrhiza has been extensively used in phytoremediation, animal feed, and biofuel production (42-44, 41). Furthermore, advances in S. polyrhiza genetics and genomics have provided insight into the molecular mechanisms of high starch and protein production and stress tolerance (45, 46, 44, 47, 41). Analysis of fatty acid and TAG levels in 30 duckweed species confirmed that TAG levels in duckweed are comparable to those in the leaves of terrestrial plants (48). Genetic engineering strategies were recently established to accumulate TAG in duckweed for industrial applications (49).

Transcriptome deep sequencing analyses (RNA-seq and other related techniques) quantitatively compare gene expression within all plant tissues and organ types across multiple species (50-53). Transcriptomic analysis is most effective when applied to plants at multiple developmental stages, when exposed to various stress conditions, or to mutant or transgenic plants with noticeable differences in their phenotypes (54-57). RNA-seq has been used to identify differentially expressed genes (DEGs) associated with heavy-metal stress in plants and microalgae (56, 58-60). RNA-seq experiments with duckweeds have been employed to further our understanding of genes involved in responses to stress induced by heavy metals (43, 47); phytohormones, extreme temperature, nutrient availability, ammonium toxicity, ionizing radiation, and carbon sources (40, 61); and salt (45, 62).

FGD wastewater generated from coal-fired power plants contains several heavy metals, with arsenic (As), cadmium Cd), chromium (Cr), copper (Cu), lead (Pb), mercury (Hg), selenium (Se), and zinc (Zn) representing the major heavy metals ((63); see below table). In this study, we conducted time-course experiments to investigate the lipidomic and transcriptomic changes in S. polyrhiza in response to heavy-metal stress induced by FGD wastewater. Our results suggest that heavy-metal stress promotes the degradation of chloroplast lipids and the accumulation of TAGs via active membrane lipid modification. Our transcriptomic data suggest that TAG and starch biosynthesis are independent processes in S. polyrhiza, unlike in unicellular photosynthetic organisms. Additionally, we characterized the role of SpWRINKLED3 (SpWRI3), one of the top upregulated genes under FGD wastewater treatment and a member of the APETALA2/ETHYLENE-RESPONSE FACTOR (AP2/ERF) transcription factor family, in tolerance to FGD wastewater-induced heavy-metal stress. These results uncover the underlying mechanisms of heavy-metal stress responses and lay the foundation for improving phytoremediation or the accumulation of bioproducts (biodiesel or starch) in S. polyrhiza.

| Composition of flue gas | | |
|---|---|---|
| desulfurization (FGD) | | |
| wastewater | | |
| Constituents | Concentration | |
| Aluminum | 0.248 | mg/L |
| Arsenic | <0.0050 | mg/L |
| Boron | 268 | mg/L |
| Cadmium | 0.046 | mg/L |
| Calcium | 865 | mg/L |
| Cobalt | 0.857 | mg/L |
| Copper | 0.016 | mg/L |
| Iron | 0.14 | mg/L |
| Mercury | 0.39 | μg/L |
| Lead | <0.005 | mg/L |
| Magnesium | 3320 | mg/L |
| Manganese | 39.3 | mg/L |
| Nickel | 0.463 | mg/L |
| Potassium | 51.5 | mg/L |
| Sodium | 120 | mg/L |
| Strontium | 9.31 | mg/L |
| Zinc | 0.353 | mg/L |
| pH | 7.17 | |
| ITSS | 1238 | mg/L |
| TDS | 10400 | mg/L |
| COD | 692 | mg/L |
| TSS: Total suspended solids | | |
| TDS: Total dissolved solids | | |
| COD: Chemical oxygen demand | | |

### Materials and Methods

### Duckweed Strains and Culture Conditions

S. polyrhiza wild-type strains were obtained from the Rutgers Duckweed Stock Cooperative at Rutgers University. Eight to ten frond colonies were inoculated with three replicates in Magenta jars containing 80 mL of liquid Schenk and Hildebrandt (SH) salts [0.5×(1.6 g/L) Sigma Cat. #S6765; PhytoTechnology Laboratories Cat. #S816)] with 0.5% sucrose and were cultured for 13 days in a plant growth chamber (Percival, USA) under a 16-h-light/8-h-dark photoperiod with a light intensity of 80 μmol m⁻² s⁻¹ at 25° C. To investigate the physiological changes in S. polyrhiza in response to FGD wastewater-derived heavy-metal stress, after about 13 days of culture, 200 freshly grown plantlets were placed in Magenta jars containing SH nutrient medium (0 h control), tap water (T), or FGD wastewater (F), each in six replicates. The cultures were incubated in a plant growth chamber under a 16-h-light/8-h-dark photoperiod with a light intensity of 80 μmol m⁻² s⁻¹ at 25° C.

To investigate lipid and carbohydrate contents and perform transcriptome analysis, S. polyrhiza was cultured under the respective treatment conditions as described above. Samples were collected with six replicates at 0, 24, 48, and 72 h into treatment. All samples were immediately frozen in liquid nitrogen and stored at −80° C.

### Plant Collection and Lipid Extraction

Whole S. polyrhiza plantlets were collected from a 13-day-old culture (0 h) grown in Magenta jars on a culture medium and plants were treated in tap water (control; T) and FGD wastewater (F) at 24, 48, and 72 h for transmission electron microscopy (TEM). Plants were transferred immediately into 3 mL isopropanol with 0.01% (v/v) butylated hydroxytoluene (BHT; Sigma Aldrich B1378) that was pre-heated for 15 min at 75° C. in a 30-mL glass test tube with a Teflon-lined screwcap (DWK Life Sciences, Millville, NJ, USA). For lipid extraction, S. polyrhiza plantlets were collected from 13-day old cultures (0 h) and grown in Magenta jars containing SH medium with 0.5% (w/v) sucrose, tap water or FGD wastewater for 24, 48, or 72 h. All samples were immediately frozen in liquid nitrogen and stored at −80° C.

Once plantlets were placed in isopropanol, they were incubated at 75° C. for 15 min to deactivate their lipid-hydrolyzing enzymes. The tubes with plantlets were cooled to room temperature, and then 1.5 mL chloroform and 0.6 mL HLPC-grade water were added to each tube. The glass tubes were stored at −80° C. For further analysis, the glass tubes were allowed to reach room temperature and shaken on an orbital shaker for 1 h at room temperature. The solvents in each glass tube were transferred by Pasteur pipette to a new glass tube while leaving the plants in the original tube. A 4 mL aliquot of chloroform:methanol (2:1; v/v) with 0.01% (v/v) BHT was added to each original tube, and the glass tubes containing plantlets were shake overnight on an orbital shaker at room temperature. The solvent was combined with the first extract and constituted the lipid extract.

The addition of chloroform:methanol with 0.01% (v/v) BHT, followed by overnight shaking and transfer of the solvent into the tube containing the first extract was carried out four times. Subsequently, the solvent in lipid extracts was evaporated using an N-EVAP 112 nitrogen evaporator (Organomation Associates, Berlin, MA, USA). The lipid extract was dissolved in 1 mL chloroform and transferred to a 2-mL clear-glass vial with a Teflon-lined screwcap (DWK Life Sciences, Millville, NJ, USA). These vials were stored at −80° C. until preparation for shipping to the Kansas Lipidomics Research Center (KLRC) for lipid profiling. Before shipping, the solvent (chloroform) was again evaporated from the 2-mL vials using the N-EVAP 112 nitrogen evaporator. At KLRC, the lipid extracts were dissolved in 1 mL chloroform and subjected to lipid profiling. The whole plantlets in glass tubes were dried overnight at 105° C., and their dry weights were measured. Dry weights were determined using a microbalance (AX26, Mettler Toledo, Columbus, OH, USA) with a detection limit of 2 μg.

### Cuticular Wax Extraction and Analysis

Cuticular waxes were extracted from lyophilized duckweed plantlets by submersion in methylene chloride for a period of 30 s, followed by filtration through solvent rinsed Whatman #4 qualitative filter paper to remove plant material from the extracts. The following internal standards (5 μg each) were added to each extract: heptadecanoic acid (17:0 FFA), octacosane (28:0 n-alkane), and triacontan-1-ol (23:0 1-Alc). Extracts were stored at −20° C.

For analysis, wax extracts were allowed to warm to room temperature and evaporated under nitrogen gas, followed by gentle warming (35° C.) to dryness. The dried extracts were silylated using a 1:1 (v/v) mixture of N,O-bis (trimethylsilyl) trifluoroacetamide with trimethylchlorosilane (99:1) and anhydrous pyridine. Derivatized samples were gently evaporated under nitrogen gas to remove excess derivatizing agent and dissolved in n-heptane for GC-MS analysis.

Wax samples were analyzed on an Agilent 7890 gas chromatograph equipped with an auxiliary electronic pressure controller (auxEPC) and two-way splitter that directed column effluent to both a flame ionization detector (FID) and an Agilent 5977 mass spectrometer (MS). The following parameters were used for GC-MS-FID analysis: 1 μL of the sample was introduced to a split/splitless inlet programmed for splitless injection set to a temperature of 320° C. Column flow was set to 1.5 mL/min. The oven temperature program was 120° C. for 3 min, followed by a linear ramp at 10° C. per min to 325° C. for 10 min.

Wax constituents were quantified based on constituent peak areas relative to internal standard peak areas. Peak areas were corrected by applying theoretical FID response factors, which assume that the FID response is proportional to carbon mass for all carbons bonded to at least one H-atom (64). Constituents were identified based on their mass fragmentation patterns using in-house developed mass spectral libraries or the NIST/EPA/NIH 17 (National Institute of Standards and Technology, Gaithersburg, MD, USA) mass spectral library.

### Lipid Profiling Using an Electrospray Ionization Triple Quadrupole Mass Spectrometry

For lipidomics analysis, S. polyrhiza plantlets were collected from 13-day-old cultures (0 h) and grown in Magenta jars containing SH medium with 0.5% (w/v) sucrose, tap water, or FGD wastewater for 24, 48, or 72 h. The plantlets were subjected to lipid extraction as described above. TAGs and DAGs were analyzed in three biological replicates using electrospray ionization triple quadrupole mass spectrometry with a series of neutral loss scans for specific fatty acids and polar lipoids, which were acquired on a Waters Xevo TQS mass spectrometer (Waters Corporation, Milford, MA, USA). Instrument parameters are shown in Supplementary Table S2 (which is incorporated herein by reference from Muthan et al., J of Hazardous Materials 469 (2024) 133951 which can be found in the online version at doi:10.1016/j.jhazmat.2024.133951). Tag data were normalized to a TAG_17:1/17:1/17:1 (NuChek Prep, Elysian, MN, Catalog #D-146) internal standard and DAG data to a DAG_15:0/15:0 (NuChek Prep, Elysian MN, Catalog #D-146) internal standard. Data were processed using the LipidomeDB Data Calculation Environment (66, 67. The identities of TAG molecular species, with the three acyl chains specified, were calculated from individual neutral loss scans using an approach similar to that of Li et al. (2014), except that no response factors were applied. Polar lipids were analyzed as previously described (68) on an Applied Biosystems 4000 Q-Trap (Sciex, Framingham, MA, USA) in three biological replicates. Unsaturation indices were calculated as the sum of the (amount of each lipid molecular species times the number of total C—C double bonds in the species) divided by the (total amount of lipid in the class times the number of acyl chains per molecule).

### Transmission Electron Microscopy

Frond samples were collected from S. polyrhiza cultures treated in tap water (control; T) or FGD wastewater (F) at 24, 48, and 72 h for TEM. Whole leaf samples were fixed in a combination of 2.5% (w/v) glutaraldehyde and 2.5% (w/v) paraformaldehyde in 0.1 M cacodylate buffer at 4° C. for 24 h, post-fixed in 1% osmium tetroxide (w/v) and dehydrated in a graded acetone series. The samples were infiltrated and embedded in Spurr resin (Polysciences). Thin microtome sections were imaged under a JEOL 100CX transmission electron microscope at a 100-kV accelerating voltage at the Center for Advanced Microscopy, Michigan State University as described (69).

### RNA Extraction, RNA-Seq, and Bioinformatics Analysis

Total RNA was extracted from three biological samples of S. polyrhiza grown on SH medium with 0.5% (w/v) sucrose (0 h control), tap water, or FGD wastewater for 24, 48, or 72 h using an RNeasy Plant Mini Kit (Qiagen, Valencia, CA) and stored at −80° C. The total RNA was measured using a Qubit assay kit (Thermo Scientific, USA), and sample integrity was assessed using a TapeStation system (Agilent, Santa Clara, CA; HS RNA kit). Libraries were created for three biological samples of S. polyrhiza plantlets treated with tap water (control) or FGD wastewater for 24, 48, or 72 h using an Illumina TruSeq Stranded mRNA Library Kit. Each library was uniquely barcoded, and equimolar amounts of each library were combined to create a library pool that was quantified with a KAPA Biosystems Illumina Library Quantification cPCR Kit. Single-end 50-bp sequencing was performed at the Michigan State University Research Technology Support Facility Genomics Core using an Illumina HiSeq 4000 instrument. The resulting RNA-seq data were used for DEG identification. Trimmomatic version 0.32 (70) was used to remove low-quality reads and adapter sequences from raw reads; the quality of the clean reads was evaluated with FASTQC (71). Cleaned reads were mapped against the reference genome (44) using STAR v2.5.2b (72). Rsubread version 3 (73) (featureCounts function) was used to build a read count matrix that was imported into R for DEG analysis using the linear model based Limma Voom package (74). Gene expression values were converted into normalized reads per kilobase per million mapped reads (RPKMs) for additional statistical analyses. Results were corrected for multiple comparisons, and adjusted P-values below a false discovery rate (FDR) threshold of 0.05 were considered to be significant. DEGs were identified between the treatment (FGD) and control (tap water) groups across three sampling time points (24, 48, and 72 h). Sequence data described in this article are available at GenBank under BioProject PRJNA891359.

### Quantitative RT-PCR

Total RNA was extracted from 12-day-old Arabidopsis seedlings or S. polyrhiza grown on SH medium with 0.5% sucrose (0 h control), tap water, or FGD wastewater at 24, 48, and 72 h using an RNeasy Plant Mini Kit (Qiagen, Valencia, CA), and the samples were stored at −80° C. Approximately 4.5 μg total RNA was reverse transcribed into first-strand cDNA using Revert Aid M-MuLV reverse transcriptase enzyme (Fermentas, USA) according to the manufacturer's protocol. The resulting cDNA (32 ng per reaction) was used as a template in a 20-μL PCR consisting of forward and reverse gene-specific primers and 2×PowerUp SYBER Green Master Mix (Applied Biosystems) on a QuantStudio 7 Flex system (Applied Biosystems). The relative transcript fold-change was calculated using the 2^{-ΔΔ}CT formula, and the S. polyrhiza ACTIN2 (Sp20g00413) or Arabidopsis ACTIN2 gene was used as the internal control. The relative expression level (as fold-change) was scaled such that the expression level of the 0 h control was set to 1.

### ¹⁴C-Acetate Radiolabeling Assay

In vivo ¹⁴C-acetate pulse-chase assay was performed as described by (75). Briefly, rapidly growing plantlets were incubated with ¹⁴C-acetate for 1 h in the light (60 μmol m⁻² s⁻¹) with shaking in 10 mL medium consisting of 1 mM unlabeled acetate, 20 mM MES pH 5.5, one-tenth strength Murashige and Skoog (MS) salts, and 0.01% (v/v) Tween 20. The assay was started by adding 0.1 mCi of ¹⁴C-acetate (106 mCi/mmol; American Radiolabeled Chemicals, St. Louis, MO, USA). At the end of the incubation, the plantlets were washed three times with water and blotted onto filter paper. For the chase period, plantlets were incubated in the same medium but lacking ¹⁴C-acetate. Total lipids were extracted from the samples as described by (75). Polar and neutral lipids were separated on a single thin-layer chromatography (TLC) plate using double development (2/3 in acetone-toluene-water [91:30:3, v/v/v], followed by hexane-diethyl ether-acetic acid [70:30:1, v/v/v]). Label incorporation into total lipids was determined by phosphor imaging using a Typhoon FLA 7000 imager (GE Healthcare). Relative radioactivity associated with individual lipids was quantified using ImageQuant5.2.

### Arabidopsis Transformation

The binary vector containing the full-length coding sequence of SpWRI3 (Sp11g00856) under the control of the Arabidopsis UBIQUITIN 10 promoter (UBQ10:SpWRI3) was introduced into Agrobacterium (Agrobacterium tumefaciens) strain GV3101 by electroporation, followed by transformation into Arabidopsis accession Columbia (Col 0) by the flower-dip method (76). Arabidopsis wild-type (Col 0) seeds or UBQ10: SpWRI3 transgenic seeds were surface sterilized and plated onto half-strength MS agar plates containing 1% (w/v) sucrose alone or with 20 mg/L hygromycin-B. After 2 days of stratification at 4° C. in the dark, the plates were moved into a plant growth chamber set to a 16-h-light/8-h-dark photoperiod (70-80 μmol m⁻²s⁻¹) at 22° C. Twelve-day-old wild-type and transgenic seedlings were transferred to soil (Promix, BFG Supply Co., USA) and grown in a plant growth chamber as mentioned above. For experimental purposes, wild-type and homozygous transgenic seedlings/plants were grown on the same shelf of the plant growth chamber in a randomized design when used for gene expression analysis and FGD wastewater treatment.

### Estimation of Total Chlorophyll, Reduced Glutathione, and Carbohydrate Contents

Chlorophyll and reduced GSH were extracted from S. polyrhiza (50 mg fresh weight [FW]) grown on SH medium with 0.5% sucrose (0 h), tap water, or FGD wastewater for 24, 48, and 72 h. Whole plantlets were ground in 1 mL 80% (v/v) acetone and incubated at −20° C. for 1 h in the dark. Total chlorophyll analysis was carried out as described previously (77). GSH was quantified using a glutathione assay kit following the manufacturer's instructions (Sigma-Aldrich, catalog number: CS0260). Whole S. polyrhiza plantlets grown on nutrient medium (0 h), tap water, or FGD wastewater for 24, 48, and 72 h were freeze-dried, ground into a fine powder in a Retch Mill, and used for carbohydrate analysis as described (78, 79). 3- to 4-week-old wild-type and UBQ10:SpWRI3 transgenic Arabidopsis plants treated with tap water or FGD wastewater were homogenized in trichloroacetic acid (TCA, 0.5%, w/v) prior to analysis.

Detection of Superoxide Radicals and Hydrogen Peroxide (H₂O₂) by Histochemical Staining

3- to 4-week-old seedlings grown on tap water and FGD wastewater were randomly selected. In situ visualization of superoxide radicals using nitroblue tetrazolium (NBT) staining was previously described (80). For the detection of H₂O₂ using DAB staining, seedlings grown on tap water and FGD wastewater at 72 h were immersed in freshly prepared 0.1% (w/v) 3,3-diaminobenzidine (DAB) (pH 3.8), followed by vacuum infiltration and incubation in dark at 22° C. for 4.5-5 h. After incubation, seedlings were placed in acetic acid:glycol:ethanol (1:1:3 w/v) solution at 95° C. for 10 min. The samples were stored in 95% ethanol until photographed (81).

### Estimation of Malondialdehyde (MDA) Content

Samples (around 50 mg FW) collected from 3-4 weeks-old wild-type (WT) and Ubi10::SpWRI3 transgenic Arabidopsis plants treated in FGD wastewater were homogenized in trichloroacetic acid (TCA, 0.5%, w/v). Samples were centrifuged at 12,000 rpm for 10 minutes at 4° C. The pellet was dissolved in 1 ml of 0.5% thiobarbituric acid (TBA) in 20% TCA. Then samples were incubated at 80° C. for 30 minutes, followed by a quick chill on an ice and centrifuged at 12,000 rpm for 5 minutes at room temperature. Absorbance of supernatant was measured at 450, 532 and 600 nm and 0.5% TCA solution used as a blank. The maximum TBA-MDA complex absorbance (A532) and the nonspecific turbidity absorbance (A450 and 600) were used to determine the MDA contents at an extinction coefficient of 155,000 M⁻¹ cm⁻¹ previously described by (Heath and Packer, 1968; Hodges et al., 1999).

3,3-diaminobenzidine (DAB) and Nitro Blue Tetrazolium (NBT) Staining

Samples (˜50 mg FW) collected from 3- to 4-week-old wild-type (WT) and UBQ10:SpWRI3 transgenic Arabidopsis plants treated with tap water or FGD wastewater were homogenized in trichloroacetic acid (TCA, 0.5%, w/v). The samples were centrifuged at 14,000 g for 10 min at 4° C. The pellet was dissolved in 1 mL of 0.5% (w/v) thiobarbituric acid (TBA) in 20% (w/v) TCA. The samples were incubated at 80° C. for 30 min, followed by a quick cooldown on ice, and another centrifugation at 14,000 g for 5 min at room temperature. The absorbance of the supernatant was measured at 450, 532, and 600 nm; 0.5% (w/v) TCA was used as a blank. The maximum TBA-MDA complex absorbance (A₅₃₂) and the nonspecific turbidity absorbance (A₄₅₀ and A₆₀₀) were used to determine MDA contents at an extinction coefficient of 155,000 M⁻¹ cm⁻¹ as previously described (82, 83). The concentration of MDA was measured according to the following equation: mol/liter=(OD₅₃₂−OD₆₀₀)−0.56*OD₄₅₀.

### Analysis of Major Metals, pH, TDS, TSS, and COD in FGD Wastewater

FGD wastewater was procured from Liberty Hydro LLC, South Charleston, West Virginia, USA. FGD wastewater samples were collected according to standard procedures and stored in airtight containers in refrigerators until use. FGD wastewater sample preparation and analysis of heavy metals in FGD wastewater were carried out as per EPA 200 methods as previously described (84). Metals were analyzed using inductively coupled plasma-mass spectroscopy (ICP-MS, Agilent, California, USA) at Pace Analytical Service, Hurricane, West Virginia, USA. The pH of the FGD wastewater was measured using a benchtop pH meter (Mettler Toledo, USA). Total dissolved solids (TDS) were measured using a benchtop multi-parameter meter (HQ440D, HACH Company, Colorado, USA). Total suspended solids (TSS) were measured using a Turbidity, Suspended Solids, and Sludge Level system (LXV 322.99.00002, HACH Company, Colorado, USA). Chemical oxygen demand (COD) was measured as described previously (85) using a digital reactor (DRB200, HACH Company, Colorado, USA).

### Arabidopsis Leaf TAG Quantification by Chromatography

Neutral lipids were extracted from aerial biomass of Arabidopsis plants treated with FGD wastewater or half-strength MS liquid medium using chloroform/methanol/formic acid (1:2:0.1, by volume). An equal volume of lipids was loaded, dried, and separated on silica plates (Silica Gel 60, EMD Millipore Corporation) by thin-layer chromatography (TLC) in a solvent consisting of hexane/diethyl ether/acetic acid (70:30:1, by volume). The TLC plates were dried thoroughly to remove traces of solvents, and TAG bands were visualized using iodine vapor. Relative TAG band intensity was measured using ImageJ software.

### Starch Analysis

Accumulation of starch in 3-week-old Arabidopsis plants was visualized by Lugol's iodine staining reagent (Sigma, USA). Whole Arabidopsis plants were harvested at the end of the day and blanched in 80% (vol/vol) ethanol. After several rounds of washing with distilled water, the whole plants were stained with Lugol's reagent for 6 h and destined with water. Stained whole plants were photographed with an Olympus SZX16 stereo microscope and digital camera.

### Statistical Analysis

The experimental design used in this study was a randomized complete block design. All experiments and data collection were performed with three biological replicates. The data from each experiment were analyzed separately for physiological, lipidomics, biochemical, and transcriptomic experiments. Statistical significance was assessed using the Student's pairwise t-test. Data are expressed as average±SD; differences at P<0.05, P<0.01, and P<0.001 were considered to be statistically significant.

### Example 2: Physiological Changes of S. polyrhiza in Response to FGD Wastewater

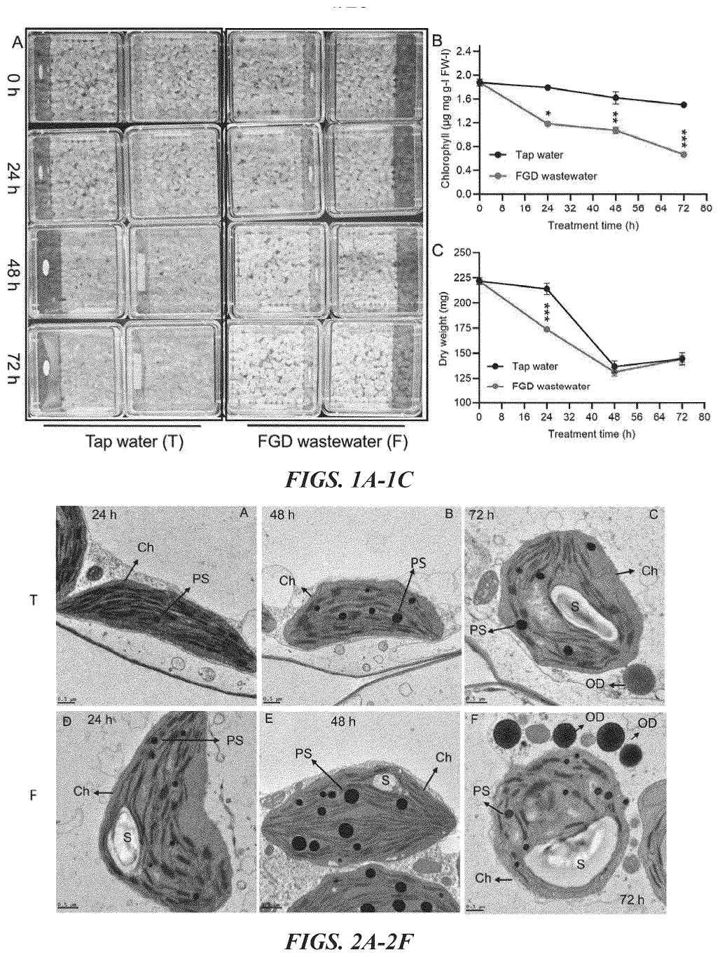

To study the physiological changes in S. polyrhiza in response to FGD wastewater, we grew S. polyrhiza cultures in liquid Schenk and Hildebrandt (SH) medium supplemented with 0.5% sucrose and under optimum growth regimes. We then transferred 3-week-old S. polyrhiza plantlets to Magenta jars containing tap water or FGD wastewater and recorded their survival rate, plant morphology, total chlorophyll contents, and dry biomass at 24, 48, and 72 h of treatment (FIG. 1). FGD wastewater-treated plantlets showed a time-dependent decrease in their growth compared to tap water-treated plantlets (FIG. 1A). We observed that about 10-15% of FGD wastewater-treated plantlets are discolored at 48 h into treatment, with most FGD wastewater-treated fronds having turned pale green by 72 h. At the 96-h time point, all FGD wastewater-treated fronds had turned yellowish or brown. The fronds maintained in tap water showed less visible discoloration, although they lacked nutrients. Consistent with the visible discoloration of fronds, chlorophyll levels were significantly lower (35%, 33%, and 52%) in FGD wastewater relative to tap water samples at 24, 48, and 72 h of treatment, respectively (FIG. 1B). The dry weight of FGD wastewater-treated plantlets was about 17% lower at the 24-h time point compared to plantlets maintained in tap water for 24 h (FIG. 1C). The dry weight did not significantly differ between plantlets grown in FGD wastewater or tap water conditions at 48 and 72 h. Taken together, plant morphology, total chlorophyll content, and dry weight are significantly affected by FGD wastewater treatment. We thus collected S. polyrhiza at 0, 24, 48, and 72 h for subsequent experiments.

### Example 3: Oil Droplets Accumulate in S. polyrhiza in Response to FGD Wastewater

We analyzed oil droplet (OD) formation and localization in fronds treated with tap water or FGD wastewater at 24, 48, and 72 h into treatment via transmission electron microscopy (TEM). We observed few or no ODs or plastoglobules (PSs) in fronds treated with tap water or FGD wastewater at 24 and 48 h (FIG. 2A, D). By contrast, in fronds treated with tap water or FGD wastewater at 72 h, we observed electron-dense ODs that are adjacent to the chloroplasts in mesophyll cells, most likely associated with the endoplasmic reticulum (ER) (FIG. 2C, F). Importantly, the number of ODs was greater in fronds treated with FGD wastewater than those with tap water at 72 h. PSs inside chloroplasts were abundant at 48 h in both tap water- and FGD wastewater-treated fronds (FIG. 2 B, E). These observations suggest that the exposure of S. polyrhiza to either tap water or FGD wastewater treatment increases the production of ODs, PSs, and starch, but ODs and starch accumulated to a greater extent under FGD wastewater conditions. The accumulation of ODs, PSs, and starch in fronds treated with tap water implies that the culture conditions, consisting of plantlets transferred from an optimal growth medium to tap water or FGD wastewater, did not meet the full nutritional demands of S. polyrhiza for nutrients at later time points. However, the observation of more ODs and larger starch granules in FGD wastewater is notable.

### Example 4: FGD Wastewater Triggers the Accumulation of TAG and Changes in the Accumulation of Other Lipids in S. polyrhiza

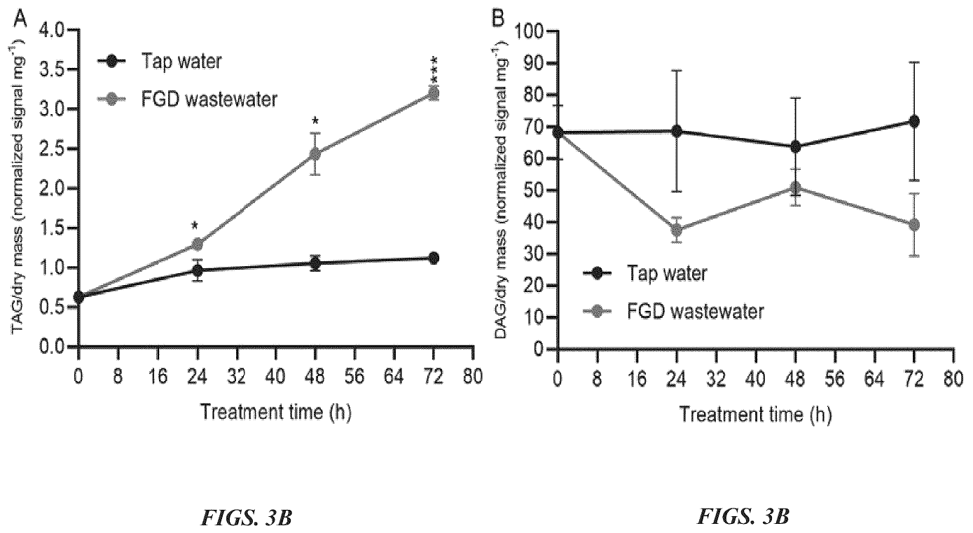

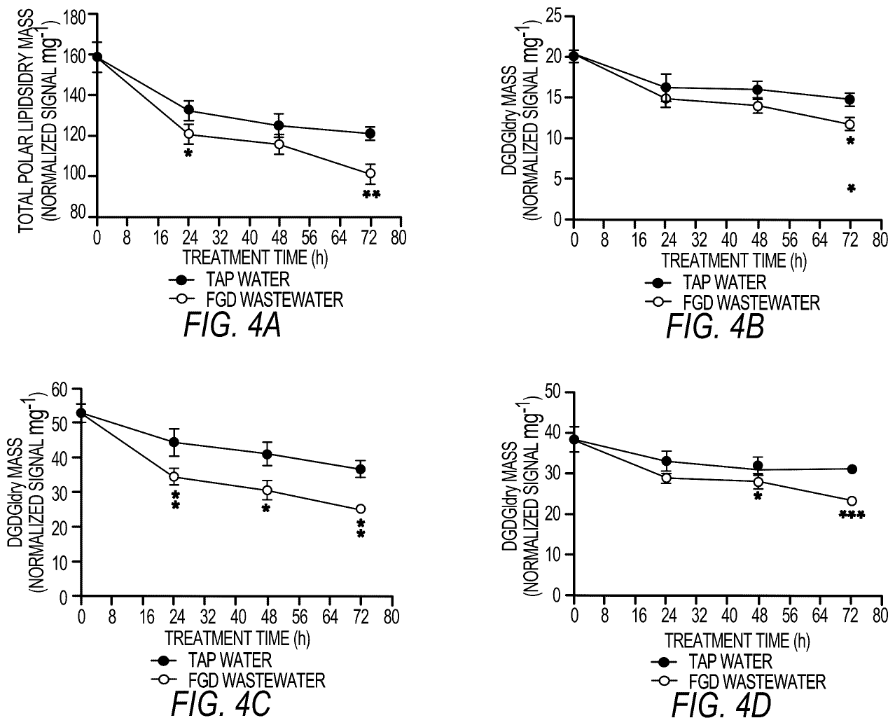

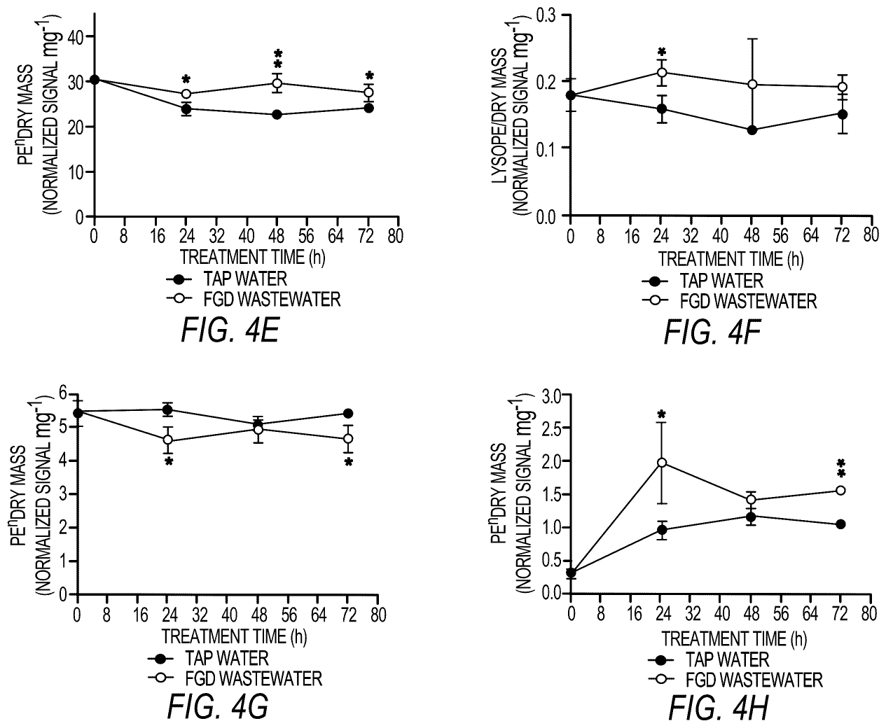

We used electrospray ionization triple quadrupole mass spectrometry (scanning neutral losses of fatty acids as ammoniated fragments) to quantify the lipids in S. polyrhiza grown in SH medium with 0.5% sucrose (0 h control), tap water, or FGD wastewater at 24, 48, and 72 h. We list the normalized mass spectral (MS) intensities for TAGs, obtained by summing data across scans for molecular species represented as total acyl carbons:total C—C double bonds in Supplementary Table S3 (which is incorporated herein by reference from Muthan et al., J of Hazardous Materials 469 (2024) 133951 which can be found in the online version at doi:10.1016/j.jhazmat.2024.133951); normalized TAG intensities from individual scans are listed in Supplementary Table S4 (which is incorporated herein by reference from Muthan et al., J of Hazardous Materials 469 (2024) 133951 which can be found in the online version at doi:10.1016/j.jhazmat.2024.133951); and TAG data with all three fatty acyl chains specified are listed in Supplementary Table S5 (which is incorporated herein by reference from Muthan et al., J of Hazardous Materials 469 (2024) 133951 which can be found in the online version at doi:10.1016/j.jhazmat.2024.133951). We detected a significantly greater increase in TAG levels in FGD wastewater-treated plantlets compared to tap water-treated plantlets, as early as 24 h into treatment (FIG. 3A). For example, the overall TAG normalized MS intensity was 1.3 times higher at 24 h and 2.9 times higher at 72 h in FGD wastewater-treated plantlets than in plantlets treated with tap water (FIG. 3A). Diacylglycerol (DAG) levels were not significantly affected by FGD wastewater treatment compared to tap water treatment (FIG. 3B, with normalized scan data shown in Supplementary Table S6 (which is incorporated herein by reference from Muthan et al., J of Hazardous Materials 469 (2024) 133951 which can be found in the online version at doi:10.1016/j.jhazmat.2024.133951) and fully specified molecular species data shown in Supplementary Table S7) (which is incorporated herein by reference from Muthan et al., J of Hazardous Materials 469 (2024) 133951 which can be found in the online version at doi:10.1016/j.jhazmat.2024.133951). An analysis of polar (membrane) lipids (FIG. 4, Supplementary Table S8, which is incorporated herein by reference from Muthan et al., J of Hazardous Materials 469 (2024) 133951 which can be found in the online version at doi:10.1016/j.jhazmat.2024.133951) showed that the accumulation of total polar lipids, as well as digalactosyldiacylglycerol (DGDG), monogalactosyldiacylglycerol (MGDG), phosphatidylcholine (PC), and PI, declined to a greater extent in S. polyrhiza subjected to FGD wastewater compared to tap water treatment (FIG. 4A-D, G). By contrast, phosphatidylethanolamine (PE) and lysoPE levels remained relatively constant in S. polyrhiza exposed to FGD wastewater compared with those in S. polyrhiza treated with tap water, which caused the levels of these lipids to decrease slightly (FIG. 4E, F). The reciprocal changes in PC and PE levels led to a decline in the PC-to-PE ratio in plantlets grown in FGD wastewater (ratio of 0.85 at 72 h) compared to tap water conditions (ratio of 1.3 at 72 h) (Fig. S1). This observation is of interest because PC is considered to be a bilayer-forming lipid, whereas PE is more likely to form a hexagonal (non-bilayer) phase. The observed sharp decrease in the PC/PE ratio could potentially cause a loss of membrane integrity at later time points under FGD wastewater conditions. Finally, the levels of PA, which may represent a biosynthetic intermediate and/or breakdown product of other membrane lipids, increased in tap water, but especially under FGD wastewater conditions (FIG. 4H).

### Example 5: Unsaturation is Higher in TAG and Lower in Most Polar Lipids in S. polyrhiza Grown in FGD Wastewater than in Tap Water

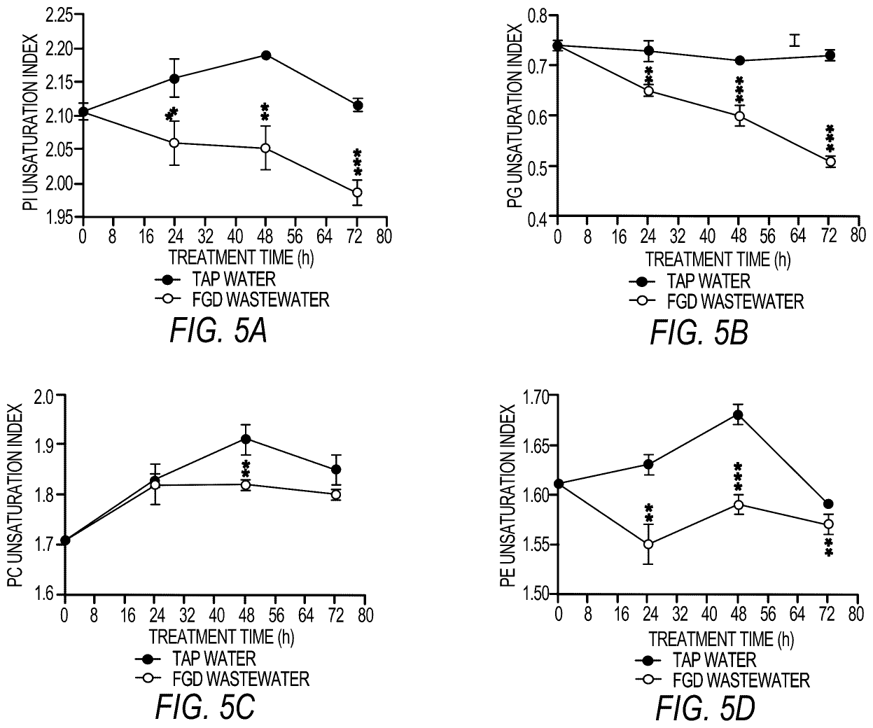

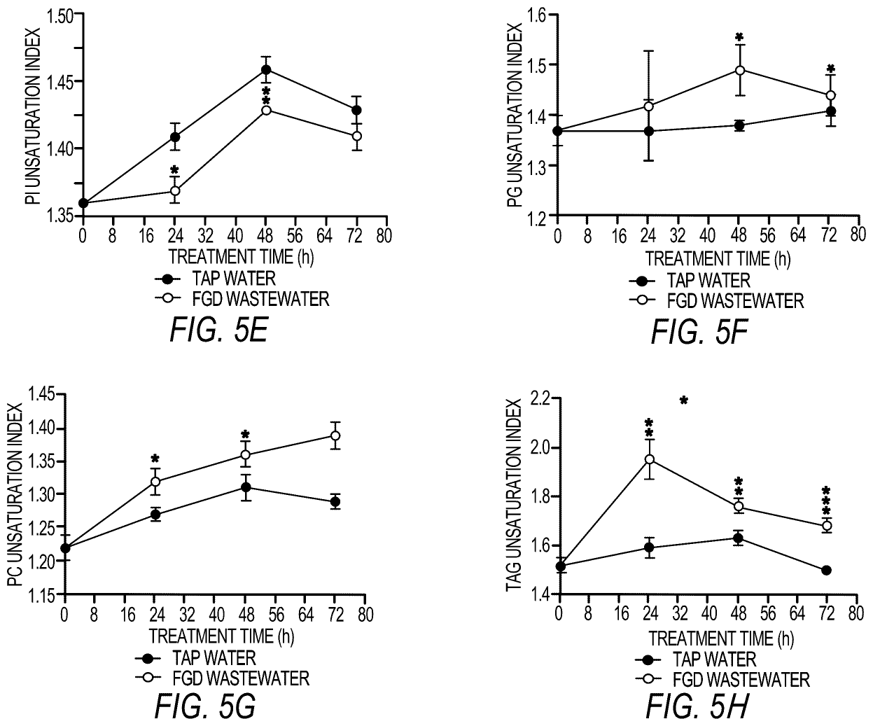

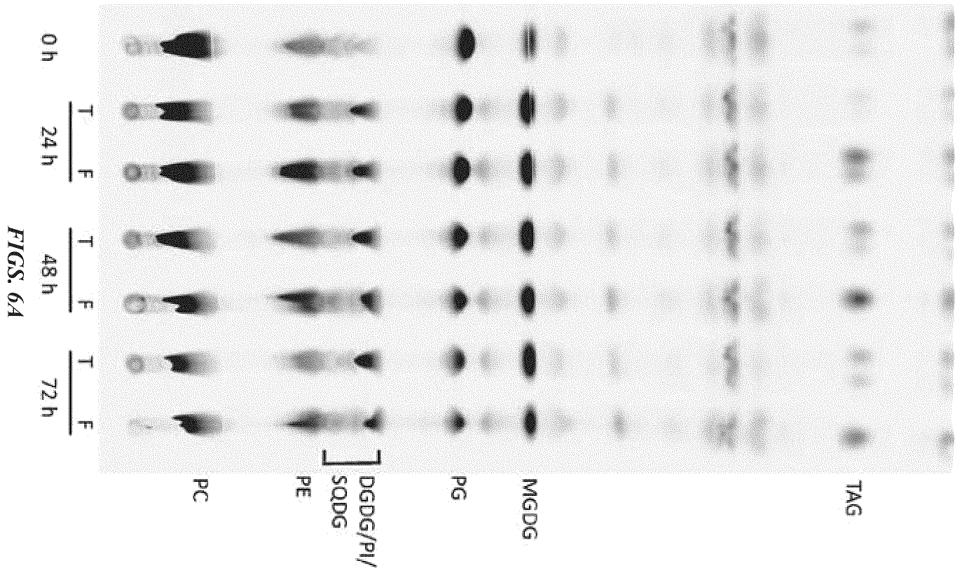

As total polar lipid levels dropped during FGD wastewater treatment (FIG. 4A), the levels of unsaturation also dropped (FIG. 5A, Supplementary Table S9, which is incorporated herein by reference from Muthan et al., J of Hazardous Materials 469 (2024) 133951 which can be found in the online version at doi:10.1016/j.jhazmat.2024.133951) from an average of 2.11 double bonds per fatty acid chain to 1.99 double bonds per fatty acid chain at 72 h of treatment. Compared to the beginning of treatment (0-h time point), tap water treatment had some effects on the level of unsaturation (FIG. 5, Supplementary Table S9). Still, compared to tap water treatment, FGD wastewater treatment resulted in lower unsaturation levels in each of the major phospholipid classes, e.g., phosphatidylglycerol (PG), PC, PE, and PI (FIG. 5B-E, Supplementary Table S9). These data suggest that polyunsaturated fatty acids are removed from these classes under FGD wastewater treatment. By contrast, the unsaturation levels of PA, DAG, and TAG increased in response to FGD wastewater treatment and were significantly higher under FGD wastewater than under tap water conditions (FIG. 5F-H, Supplementary Table S9). Whereas the TAG levels were approximately 3 times higher in plantlets treated with FGD wastewater than with tap water at 72 h of treatment (FIG. 3A, Supplementary Table S5, which is incorporated herein by reference from Muthan et al., J of Hazardous Materials 469 (2024) 133951 which can be found in the online version at doi:10.1016/j.jhazmat.2024.133951), the level of the highly polyunsaturated species TAG 18:3_18:3_18:3 was approximately 13 times higher in FGD wastewater-treated plantlets than in plantlets treated with tap water (Supplementary Table S5), indicating that this molecular species is strongly enriched in the TAG pool. These data suggest that polyunsaturated fatty acids lost from polar lipids were transferred to PA, DAG, and TAG under FGD wastewater treatment. Considering the lack of accumulation of DAG (FIG. 3B), its known metabolic functions, and the increasing unsaturation of DAG, it is reasonable to hypothesize that the observed DAG pool acts as an intermediate in the formation of TAG using a pool of fatty acids enriched in polyunsaturated fatty acids from polar lipids, including the galactolipids and perhaps PC.

### Example 6: Changes in the Radiolabeling Distribution in S. polyrhiza in Response to Tap Water and FDG Wastewater

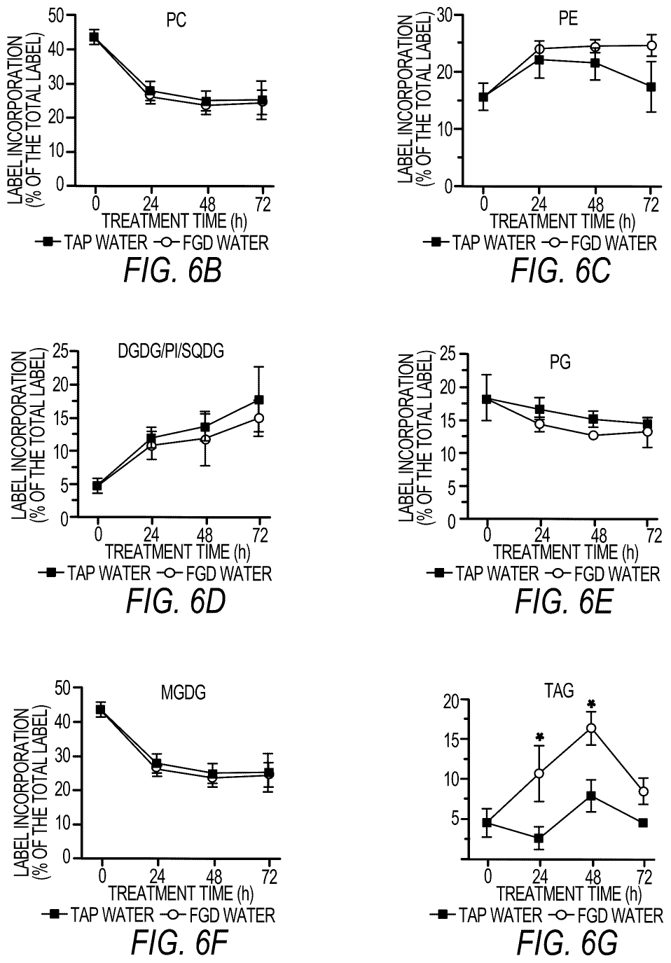

S. polyrhiza plantlets grown in SH medium supplemented with 0.5% sucrose and under optimum growth regimes were transferred to Magenta jars containing tap water or FGD wastewater to determine if this treatment altered the distribution of radiolabeled fatty acids among different lipid species. For this pulse-chase experiment, we incubated S. polyrhiza plantlets in ¹⁴C-acetate for 1 h. After washing off residual radiolabel, we divided the plantlets into two groups: plantlets incubated in tap water and plantlets incubated in FGD wastewater for 24, 48, or 72 h. We extracted total lipids from the plantlets and separated them by thin-layer chromatography (TLC) (FIG. 6A). We quantified the radioactivity associated with major membrane lipids by scintillation counting. The overall distribution of radiolabeling among major membrane lipids (PC, PE, DGDG/PI/sulfoquinovosyldiacylglycerol (SQDG), and PG) was similar between plantlets exposed to tap water or FGD wastewater conditions throughout the time course (FIG. 6B-E). However, the radioactivity associated with MGDG was significantly lower in FGD wastewater-treated plantlets at all three time points compared to tap water-treated plantlets and the 0-h control samples (FIG. 6F). By contrast, radiolabeling associated with TAG increased significantly in plantlets treated with FGD wastewater relative to plantlets treated with tap water at 24, 48, and 72 h (FIG. 6G). These observations suggest that more degraded products of MGDG, mainly polyunsaturated fatty acids (18:3), are incorporated into TAG and subsequently sequestered into lipid droplets in plantlets under FGD wastewater conditions. This result is in agreement with the overall decrease in MGDG and increase in TAG observed by lipidomics (FIGS. 3, 4).

### Example 7: Wax Composition is Altered in FGD Wastewater Treated Plants

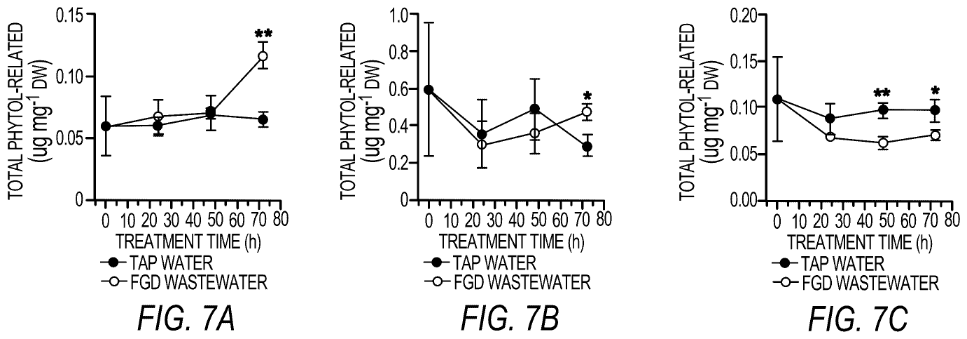

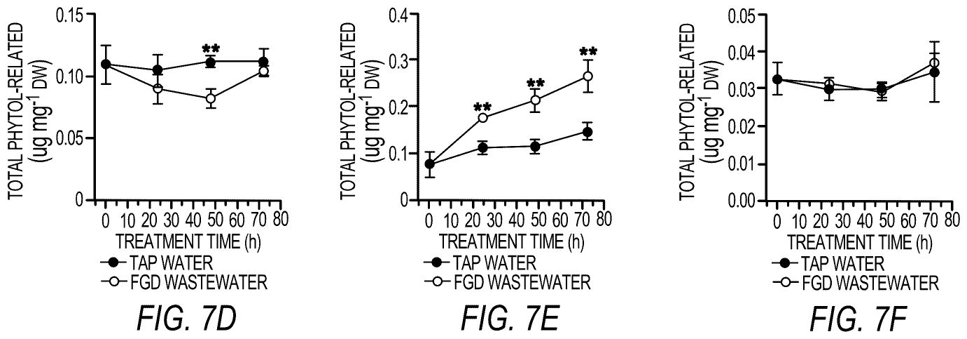

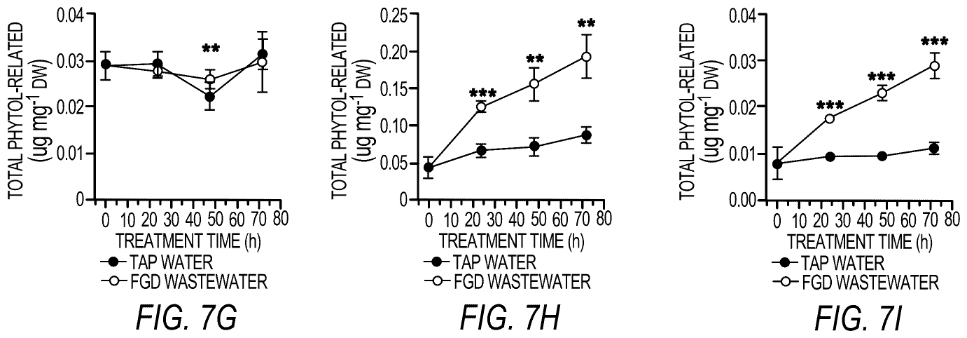

We determined the composition of S. polyrhiza wax extracts, which revealed a diverse set of aliphatic lipids, including free FFAs with carbon chain lengths ranging from 14:0-26:0, 29:1-31:1 n-alkenes, 21:0-29:0 n-alkanes, and 22:0-26:0 1-alcohols, as well as sn-1(3) monoacylglycerols (MAGs) (FIG. 7A-I). We also detected chlorophyll breakdown products such as phytol and neophytadiene in S. polyrhiza wax extracts. Triterpenoids and sterols were the dominant constituent classes present in S. polyrhiza surface lipid extracts, including squalene, campesterol, stigmasterol, β-sitosterol, isofucosterol, cycloartenol, and 24-methylene cycloartanol (Fig. S2). These results are generally in agreement with previous reports of the cuticular wax composition in S. polyrhiza fronds (86). Although FGD wastewater treatment did not significantly alter total wax content, it did affect the accumulation of specific triterpenoids and sterols (Supplementary Fig. S2). At 48 h into treatment, total sterol and triterpenoid content was 15% lower compared to tap water treatment (FIG. 7D). This difference may be attributed to lower levels of specific sterols and triterpenoids (Supplementary Fig. S2). For example, at 48 h, campesterol content was 16% lower, stigmasterol content was 6% lower, β-sitosterol content was 16% lower, cycloartenol content was 19% lower, and 24-methylene cycloartanol content was 17% lower compared to tap water treatment. The lower campesterol content persisted at 72 h with FGD wastewater treatment (36% lower than in tap water-treated plantlets). FGD wastewater treatment also led to 78% higher levels of chlorophyll breakdown products at 72 h, with neophytadiene and phytol content 156% and 26% higher, respectively, compared to tap water treatment.

FGD wastewater treatment also altered the accumulation of fatty acyl lipid cuticular waxes (FIG. 7 A, B, I). At all time points, FGD wastewater treatment led to higher amounts of total 1-alcohols (58% at 24 h, 85% at 48 h, and 81% at 72 h; P<0.01 at all time points by Student's t-test, n=3, FIG. 7E) compared to tap water. These increases may be attributed to significantly elevated amounts of 24:0 and 26:0 1-alcohols at all time points in plantlets treated with FGD wastewater relative to tap water-treated plantlets. The accumulation of sn-1(3) MAGs was also affected by FGD wastewater treatment (Supplementary Fig. S2). At 48 h, FGD wastewater-treated plantlets contained 36% less sn-1(3) MAGs than tap water-treated plantlets due to lower levels of all chain lengths of sn-1(3) MAGs (Student's t-test, P<0.05 for all MAGs, n=3). These lower amounts of total sn-1(3) MAGs (27% less) and 24:0 and 28:0 sn-1(3) MAGs persisted at 72 h into treatment with FGD wastewater (P<0.05 for all MAGs, n=3). Treatment with FGD wastewater also led to elevated amounts of FFAs at 72 h (61% more than tap water-treated plantlets; P=0.013, n=3) (Supplementary Fig. S2), including elevated amounts of 14:0, 16:0, and 18:0-24:0 FFAs at 72 h of treatment (P<0.05 for noted FFAs, n=3). FGD wastewater treatment also resulted in a 29% higher levels of 27:0 n-alkane levels at 72 h of treatment compared to tap water treatment (P=0.034, n=3) (Fig. S2).

### Example 8: Stress Imposed by Tap Water and FGD Wastewater Treatment Leads to Carbohydrate Accumulation

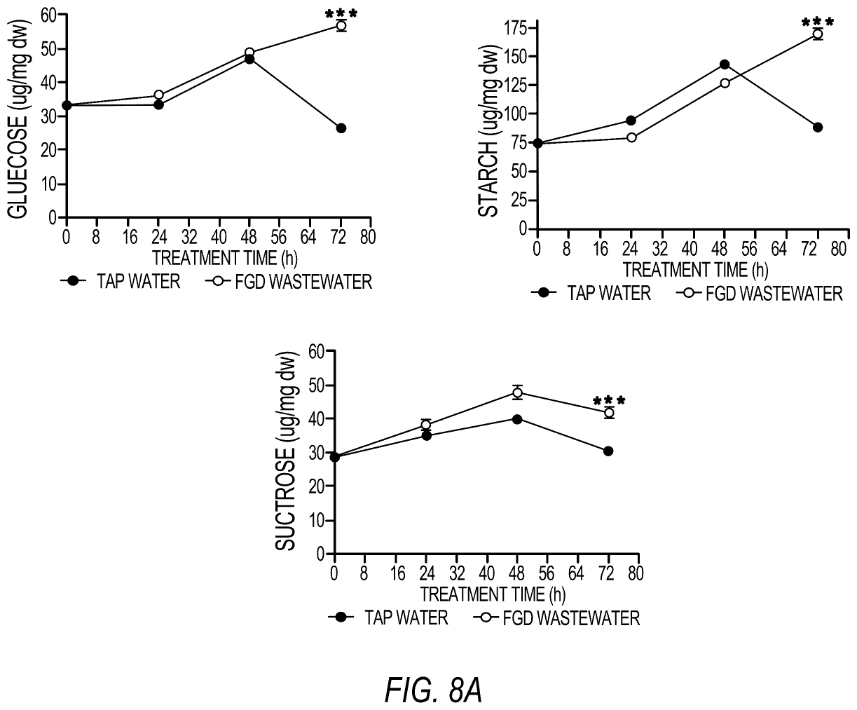

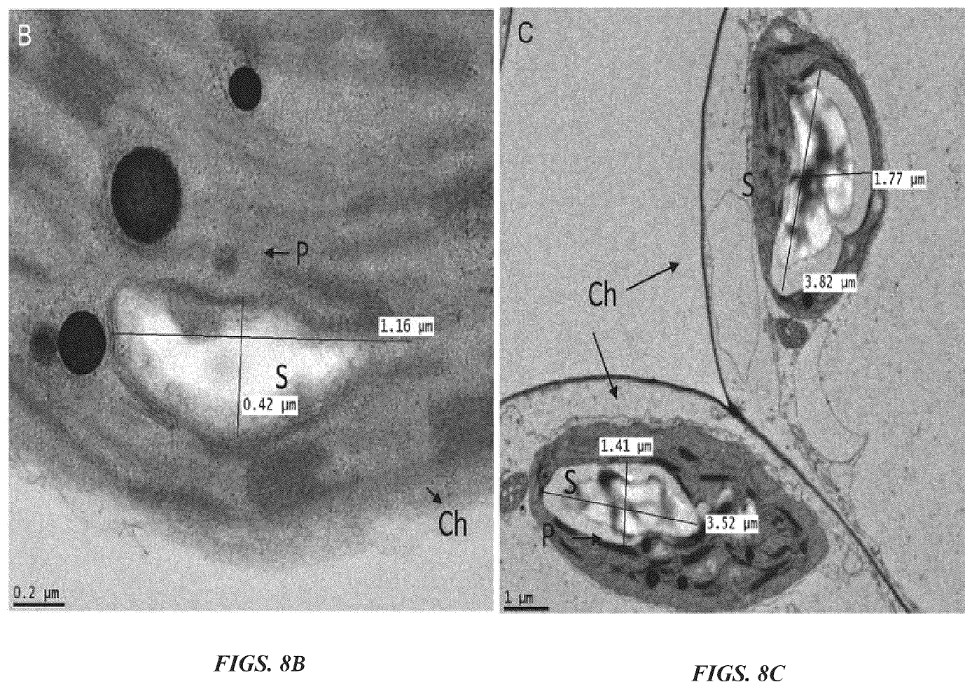

We measured carbohydrate levels in S. polyrhiza at the beginning of treatment (0 h), as well as after 24, 48, and 72 h of treatment with tap water or FGD wastewater. Both treatments showed an increase in the accumulation of glucose, starch, and sucrose in treated plantlets compared to the 0-h samples (FIG. 8A). However, at 72 h, tap water treatment was associated with a decrease in the levels of glucose, sucrose, and starch (−17%, −8%, and −9%) compared to the 0-h time point. By contrast, FGD wastewater treatment resulted in the accumulation of considerably higher levels of glucose, starch, and sucrose in S. polyrhiza at 72 h compared to the 0-h samples and plantlets treated with tap water for 72 h. We conclude that the FGD wastewater treatment induces the accumulation of carbohydrates. In addition, FGD wastewater treatment affected the size of starch granules (FIG. 2A-C). For example, at 72 h into FGD wastewater treatment, starch granule size ranged from 3.53 to 3.82 m (length) and 1.41 to 1.77 m (width) (FIG. 8C), whereas starch granules at 72 h into tap water treatment were smaller: 1.16 m in length and 0.42 μm in width (FIG. 8B). Altogether, these data indicate that exposing S. polyrhiza to FGD wastewater increases carbohydrates levels and starch granule size.

### Example 9: Upregulation of Lipid-Related Genes in S. polyrhiza

We performed an RNA-seq analysis from whole S. polyrhiza plantlets grown in SH medium supplemented with 0.5% sucrose (0 h) or transferred to tap water or FGD wastewater for 24, 48, or 72 h. After removing adapters and low-quality reads, we retained about 31.2 million clean reads, which we mapped to the S. polyrhiza reference genome (44). We used an arbitrary cutoff of 10 RPKM (reads per kilobase of exon per million fragments mapped) to consider a gene as being expressed. Genes considered for differential expression also had to have RPKM greater than 10 in more than one sample. The number of expressed samples was >1 to minimize the positive rate. To examine the differential expression of fatty acid-related genes in S. polyrhiza in response to tap water and FGD wastewater treatment, we used previously identified Arabidopsis fatty acid-related genes (87) to annotate S. polyrhiza genes.

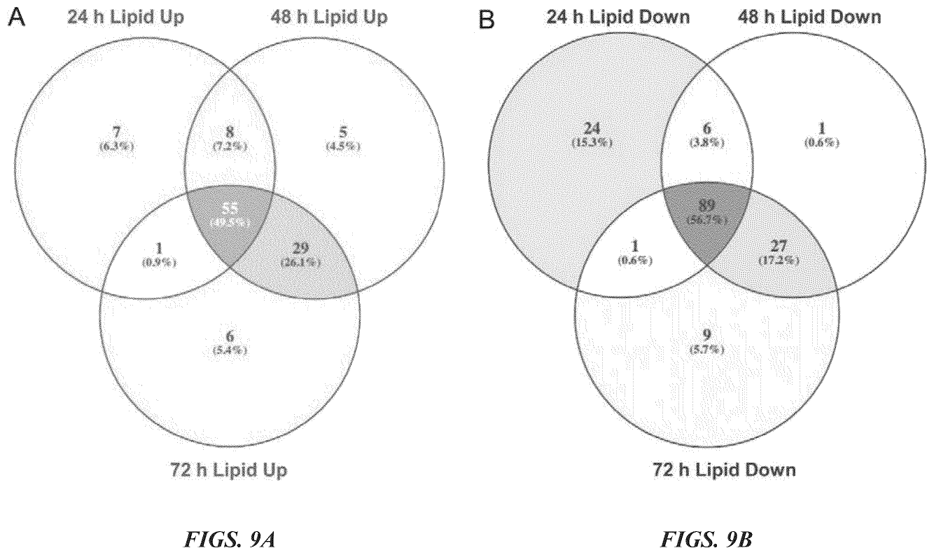

To look for changes in the expression of fatty acid metabolism genes triggered by tap water or FGD wastewater treatment, we identified differentially expressed genes (DEGs) by performing multiple comparisons (false discovery rate (FDR)<0.05) of samples collected at different time points. To simplify the dataset and concentrate on a reasonable set of genes responding to the treatments, based on FDR-adjusted P-values, we identified the top 100 upregulated or downregulated genes. We then compared the DEGs identified in samples treated with tap water to those obtained following treatment with FGD wastewater at 24, 48, and 72 h. Lipid-related genes were significantly upregulated in plantlets treated with FGD wastewater compared to those treated with tap water conditions at 24, 48, and 72 h. Notably, the upregulation of these genes was specific to FGD wastewater treatment, as the genes were downregulated in plantlets grown in tap water. Among the top 100 upregulated genes, 55 were upregulated under FGD wastewater treatments at all three time points compared to tap water treatment at the same time point (FIG. 9A). Among these 55 genes, most were related to plastidial fatty acid biosynthesis, eukaryotic phospholipid biosynthesis, sphingolipid biosynthesis, and TAG degradation. We also noticed a gene encoding a phospholipase and the transcription factor gene WRINKLED3 (SpWRI3, Sp11g00856) (Supplementary Table S10, which is incorporated herein by reference from Muthan et al., J of Hazardous Materials 469 (2024) 133951 which can be found in the online version at doi:10.1016/j.jhazmat.2024.133951) (88). In addition, 29 genes were commonly upregulated at 48 and 72 h of FGD wastewater treatment. Many of these genes were associated with eukaryotic phospholipid biosynthesis (four genes), plastidial glycerolipid biosynthesis (three genes), a phospholipase (three genes), TAG biosynthesis (two genes), cuticular wax biosynthesis (two genes), β-oxidation (one gene), and PLASTID LIPASE2 (PLIP2; Sp01g01058) (Supplementary Table S11, which is incorporated herein by reference from Muthan et al., J of Hazardous Materials 469 (2024) 133951 which can be found in the online version at doi:10.1016/j.jhazmat.2024.133951)). Only eight genes were commonly upregulated at 24 and 48 h of FGD wastewater treatment. These genes comprised two phospholipase genes, genes associated with plastidial fatty acid biosynthesis, eukaryotic phospholipid biosynthesis, mitochondrial phospholipid biosynthesis, β-oxidation, lipid trafficking, and oxylipin biosynthesis (one gene each). Only one gene was commonly upregulated at 24 and 72 h of FGD wastewater treatment. Few genes (7, 5, and 6) were specifically upregulated only at 24, 48, or 72 h of FGD wastewater treatment, respectively; most of these genes were related to wax production, phospholipid biosynthesis, and TAG degradation (Supplementary Table S11). Collectively, these data suggest that the expression of genes involved in fatty acid biosynthesis (glycerolipid, PA, TAG, and cuticular wax), degradation (TAG and MGDG), and β-oxidation is altered in S. polyrhiza in response to FGD wastewater treatment.

### Example 10: Downregulation of Lipid-Related Genes

Certain genes were significantly downregulated in plantlets treated with FGD wastewater relative to those exposed to tap water conditions for 24, 48, and 72 h. Furthermore, many downregulated genes under FGD wastewater conditions were upregulated under tap water conditions. Among the top 100 downregulated genes, 89 were commonly downregulated at 24, 48, and 72 h of FGD wastewater treatment compared to tap water conditions at the same time points (FIG. 9B). Unexpectedly, in our differential gene expression data, many genes involved in cuticular wax biosynthesis were downregulated in FGD wastewater-treated S. polyrhiza (Supplementary Table S12, which is incorporated herein by reference from Muthan et al., J of Hazardous Materials 469 (2024) 133951 which can be found in the online version at doi:10.1016/j.jhazmat.2024.133951). Thus, despite finding only a trend towards decreased total wax accumulation in FGD wastewater-treated plantlets (FIG. 7, Supplementary Fig. S2, which is incorporated herein by reference from Muthan et al., J of Hazardous Materials 469 (2024) 133951 which can be found in the online version at doi:10.1016/j.jhazmat.2024.133951), heavy-metal stress presumably inhibited the biosynthesis and accumulation of cuticular wax under FGD wastewater conditions. For example, genes encoding rate-limiting enzymes involved in cytoplasmic (ER) acyl chain elongation for cuticular wax biosynthesis, such as β-ketoacyl-CoA reductase and β-ketoacyl-CoA synthase, were downregulated under FGD wastewater treatment. Similarly, genes encoding ECERIFERUM 3, long-chain acyl-CoA synthetases, and key transcription factors involved in cuticular lipid biosynthesis, such as SHINE1 (SHN1, Sp08g01302), MYB106 (Sp03g01051), and WRI4 (Sp12g00731), were downregulated by FGD wastewater treatment (Supplementary Table S12 and S13, which are incorporated herein by reference from Muthan et al., J of Hazardous Materials 469 (2024) 133951 which can be found in the online version at doi:10.1016/j.jhazmat.2024.133951).

Several genes encoding key cuticular wax transporters were also substantially downregulated in S. polyrhiza in response to FGD wastewater treatment, such as genes encoding ATP binding cassette transporters and lipid transfer protein type 3 (Supplementary Table S12). Several genes significantly were commonly downregulated in FGD wastewater conditions at 48 and 72 h (FIG. 9B). Most of these genes were related to plastidial fatty acid biosynthesis, cuticular wax biosynthesis, and eukaryotic phospholipid biosynthesis. Only six genes were commonly downregulated after 24 and 48 h of FGD wastewater treatment, and only one gene was commonly downregulated after 24 and 72 h of FGD wastewater treatment (Supplementary Table S13). A few genes were specifically downregulated only at 24, 48, or 72 of FGD wastewater treatment. Most of the genes that were downregulated at 24 h were involved in eukaryotic phospholipid biosynthesis (Supplementary Table S13). These results indicate that the expression of lipid genes from multiple pathways, including genes related to surface lipids (wax and cuticle), plastidial fatty acid, and glycerolipid biosynthesis, is downregulated in S. polyrhiza upon exposure to FGD wastewater, thus resulting in altered lipid biosynthesis and accumulation.

### Example 11: Response of Carbohydrate Metabolism-Related Genes in S. polyrhiza

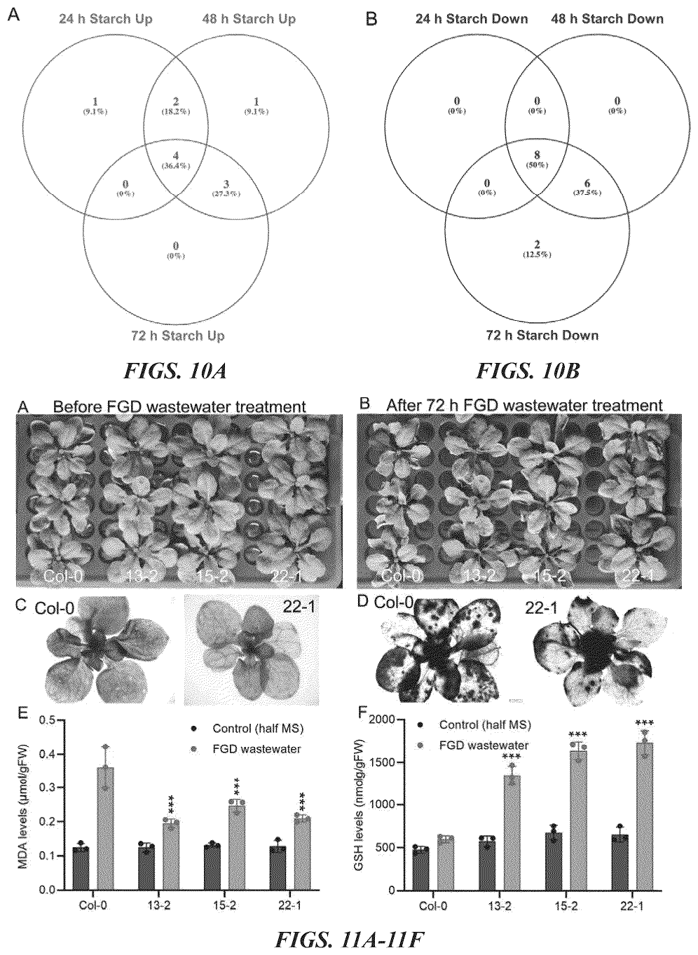

We used previously identified Arabidopsis carbohydrate-related genes to annotate S. polyrhiza genes (Zeeman et al., 2010; Streb and Zeeman 2012). Overall, starch metabolism-related genes were significantly upregulated in plantlets treated with FGD wastewater compared to those treated with tap water for 24, 48, and 72 h. Among the top upregulated genes, four were commonly upregulated by FGD wastewater at 24, 48, and 72 h compared to tap water treatment (FIG. 10A, Supplementary Table 514, which is incorporated herein by reference from Muthan et al., J of Hazardous Materials 469 (2024) 133951 which can be found in the online version at doi:10.1016/j.jhazmat.2024.133951). Three of these genes encode enzymes involved in starch degradation and a glucose transporter. Three genes were commonly upregulated at 48 and 72 h of FGD wastewater treatment. All these genes were involved in starch degradation. Only two genes were commonly upregulated at 24 and 48 h of FGD wastewater treatment. Both genes participate in starch biosynthesis. Only one gene was upregulated at 48 h of FGD wastewater treatment: APL2, encoding ADP-glucose pyrophosphorylase large subunit 2, a key enzyme required for starch biosynthesis in leaves (Supplementary Table S14). These results suggest that starch biosynthesis and degradation are simultaneously activated at an early stage of treatment with FGD wastewater; however, at later time points (48 and 72 h), starch degradation becomes predominant in plantlets.

Several genes were significantly downregulated in plantlets treated with FGD wastewater compared to tap water treatment for 24, 48, and 72 h (FIG. 10B). Most of the top downregulated genes were associated with starch biosynthesis (Supplementary Table S15, which is incorporated herein by reference from Muthan et al., J of Hazardous Materials 469 (2024) 133951 which can be found in the online version at doi:10.1016/j.jhazmat.2024.133951). Six genes were commonly downregulated at 48 and 72 h of FGD wastewater treatment. Four of these genes were involved in starch degradation and starch biosynthesis (Supplementary Table S15). These results indicate that starch biosynthesis is only active in the early stage of exposure to FGD wastewater (24 h). Genes encoding starch biosynthesis activities are repressed at 48 and 72 h into FGD wastewater treatment. However, there is a concomitant increase in starch degradation at 48 and 72 h under FGD wastewater conditions.

### Example 12: Validation of Differential Gene Expression Data by Quantitative RT-PCR

We selected 20 key genes involved in fatty acid biosynthesis, TAG biosynthesis, starch biosynthesis, and starch degradation to validate the results of DEG analysis obtained from the RNA-seq data. We isolated total RNA from whole S. polyrhiza plantlets grown in SH medium supplemented with 0.5% sucrose (0 h), or tap water or FGD wastewater for 24, 48, and 72 h and performed RT-qPCR analysis using gene-specific primers (Supplementary Table S16, which is incorporated herein by reference from Muthan et al., J of Hazardous Materials 469 (2024) 133951 which can be found in the online version at doi:10.1016/j.jhazmat.2024.133951). The RT-qPCR analysis confirmed the differential gene expression patterns observed in the RNA-seq data under tap water and FGD wastewater conditions (Fig. S3, S4). The expression trends are therefore consistent between independent measurements.

### Example 13: Physiological Characterization of Arabidopsis Plants Expressing S. polyrhiza WRINKLED3 (SpTWRI3) in Response to FGD Wastewater

In our RNA-seq analysis, under FGD wastewater conditions, we identified SpWRI3 (Sp11g00856) as one of the topmost upregulated genes (Supplementary Table S10, which is incorporated herein by reference from Muthan et al., J of Hazardous Materials 469 (2024) 133951 which can be found in the online version at doi:10.1016/j.jhazmat.2024.133951). SpWRI3 is a member of the AP2/ERF transcription factor family and a homolog of Arabidopsis WRI3 (At1g16060) (Fig. S5). RT-qPCR analysis confirmed the high levels of SpWRI3 transcript levels in S. polyrhiza treated with FGD wastewater for 24, 48, and 72 h (Supplementary Fig. S3, which is incorporated herein by reference from Muthan et al., J of Hazardous Materials 469 (2024) 133951 which can be found in the online version at doi:10.1016/j.jhazmat.2024.133951). We cloned the full-length coding sequence of SpWRI3 into a binary vector, placing its expression under the control of the Arabidopsis UBIQUITIN10 promoter. We generated multiple independent Arabidopsis transgenic lines using Agrobacterium (Agrobacterium tumefaciens)-mediated transformation and confirmed the expression of SpWRI3 in homozygous transgenic lines by RT-qPCR with gene-specific primers (Fig. S6 A, B).

Based on the expression pattern of SpWRI3 in Arabidopsis, we chose three homozygous lines (#13-2, #15-2, and #22-1) for physiological assessment. We established hydroponic cultures of the wild type (accession Col-0) and three transgenic lines in small plastic boxes containing half-strength MS basal liquid medium and grew the seedlings in a plant growth chamber at 22° C. under a 16-h-light/8-h-dark photoperiod. We transferred 3-week-old plants to a hydroponic container containing FGD wastewater or half-strength MS liquid medium (control). We imposed treatment with FGD wastewater for up to 72 h, at which point all transgenic lines appeared to be physiologically tolerant to FGD wastewater and remained green beyond 96 h. Wild-type plants wilted and ultimately died within 72 h (FIG. 11 A, B). As revealed by DAB (3,3-diaminobenzidine) and NBT (nitro blue tetrazolium) staining, SpWRI3 transgenic plants accumulated lower H₂O₂ and superoxide radicals, respectively (FIG. 11C, D). We measured the malondialdehyde (MDA) content of the plants as a measure of lipid peroxidation, which is an indicator of oxidative damage under heavy-metal stress. Under FGD wastewater treatment, MDA levels are lower in transgenic lines than those of Col-0 (FIG. 11E). GSH helps chelate toxic heavy metals and metalloids, which are subsequently transported to and sequestered in the vacuole. Under FGD wastewater treatment, SpWRI3 transgenic plants accumulated elevated levels of GSH compared to Col-0 plants (FIG. 11F). Thus, SpWRI3 overexpression leads to a higher accumulation of GSH and less lipid peroxidation under FGD wastewater treatment than the wild type. Together, these data suggest that the ectopic expression of SpWRI3 in Arabidopsis improved plant tolerance to FGD wastewater.

Furthermore, we performed TLC to investigate the accumulation of TAG in Arabidopsis Col-0 plants overexpressing SpWRI3 and wild-type Col-0 plants treated for 72 h with FGD wastewater or half-strength MS liquid medium. As shown in Fig. S7A and B, 72 h FGD wastewater caused a drastic increase in TAG accumulation in SpWRI3 transgenic lines compared to identically treated Col-0. We measured the starch content of the SpWRI3 overexpression lines and Col-0 plants as an indicator of carbohydrate accumulation. Under FGD wastewater treatment, starch levels are higher in transgenic lines than those of Col-0 (Fig. S8). These results suggest that FGD wastewater induced the accumulation of TAG and starch in SpWRI3 transgenic Arabidopsis plants, further validating lipidomics and transcriptomic data.

### Example 14: Discussion

### Increased Expression of Plastid Lipase in FGD Wastewater-Treated Duckweed

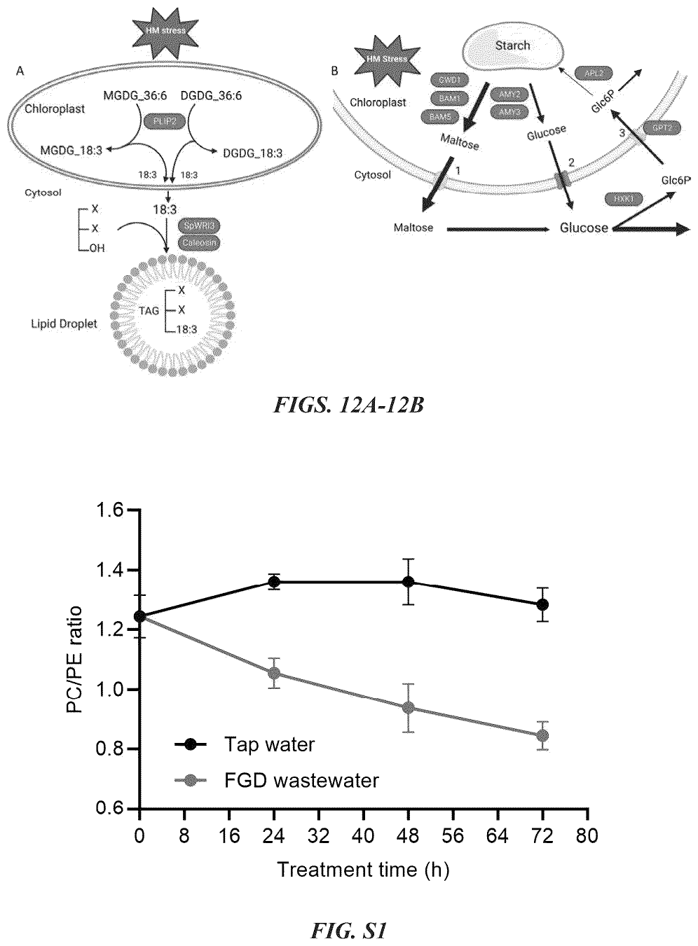

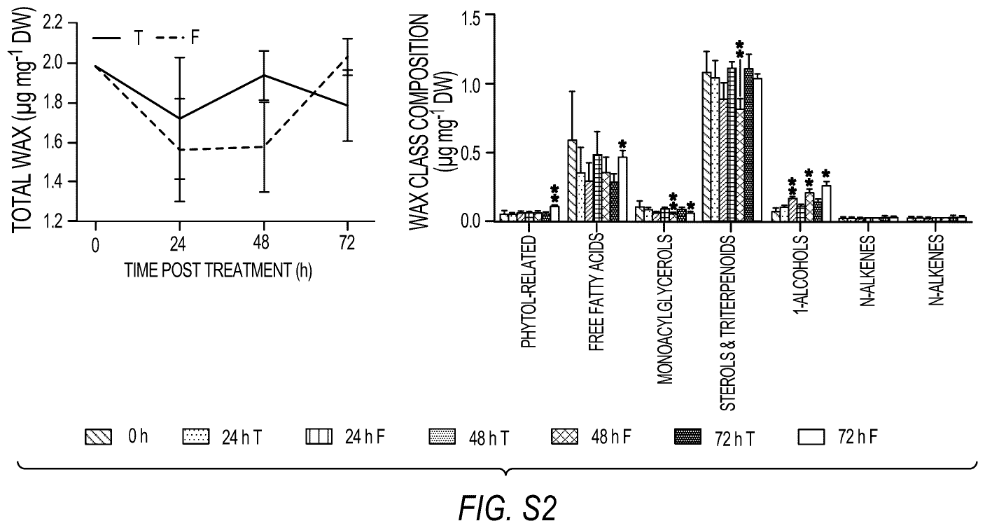

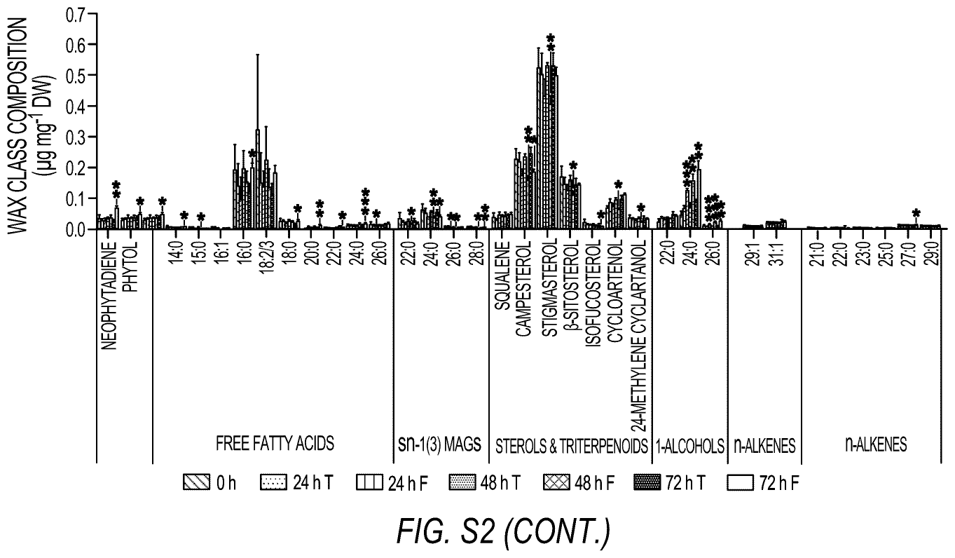

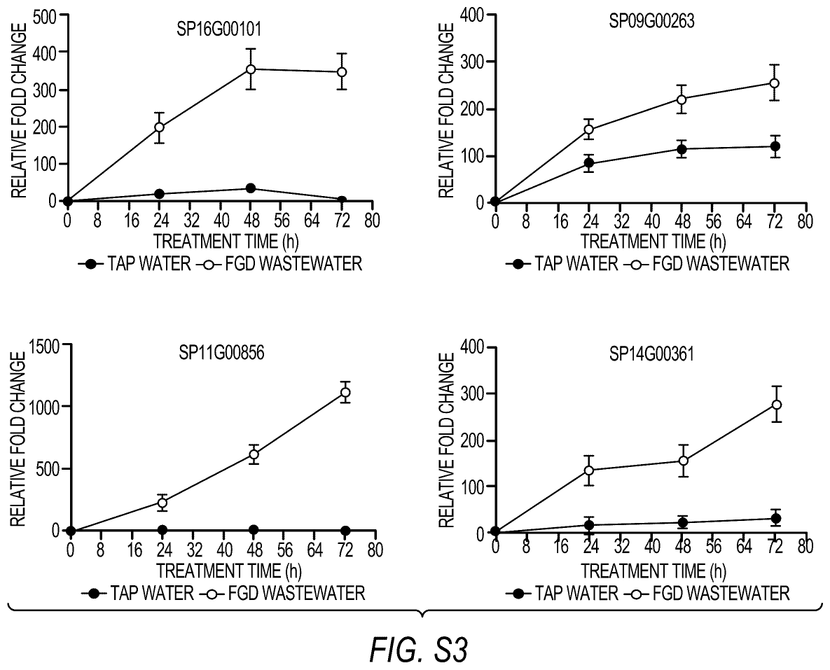

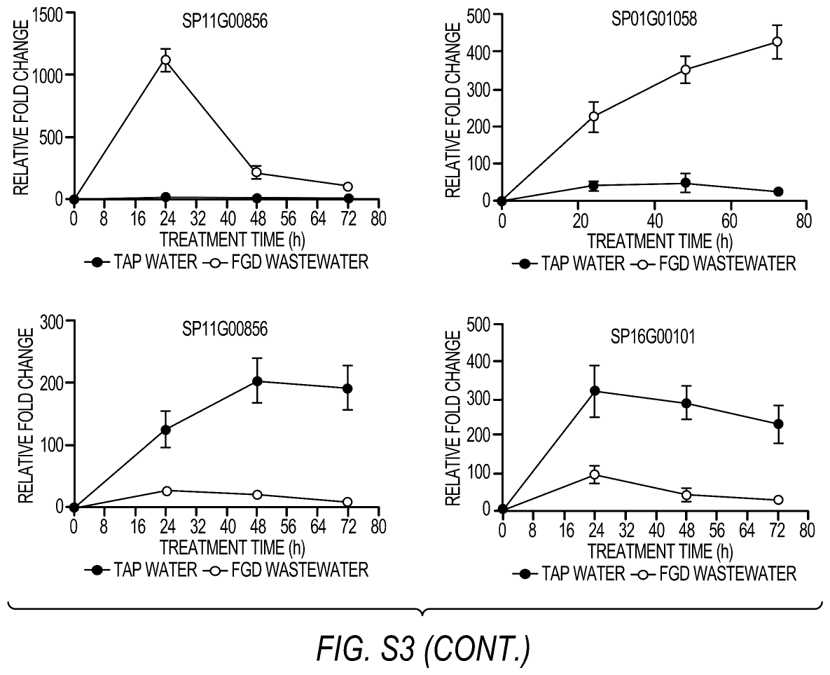

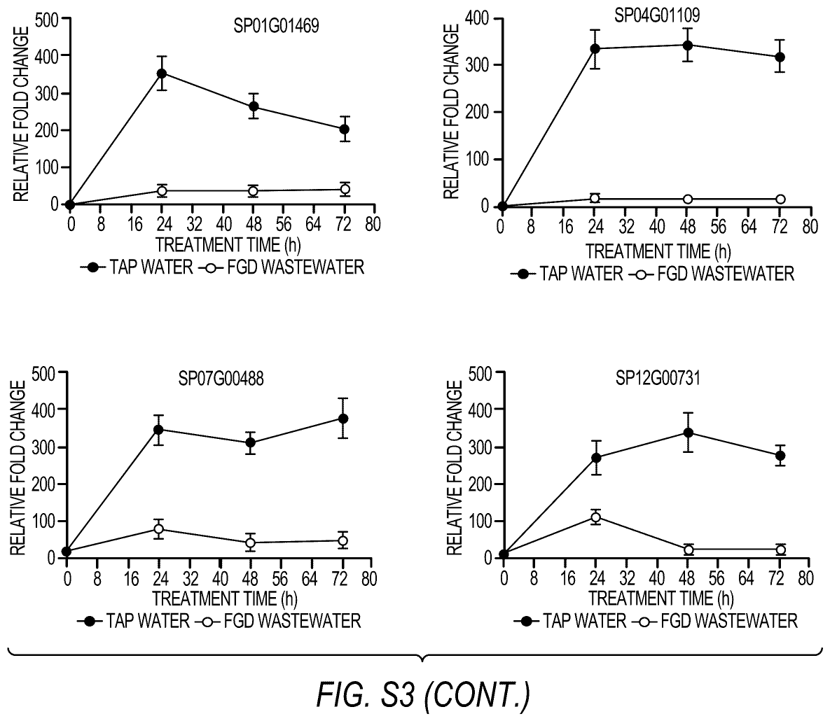

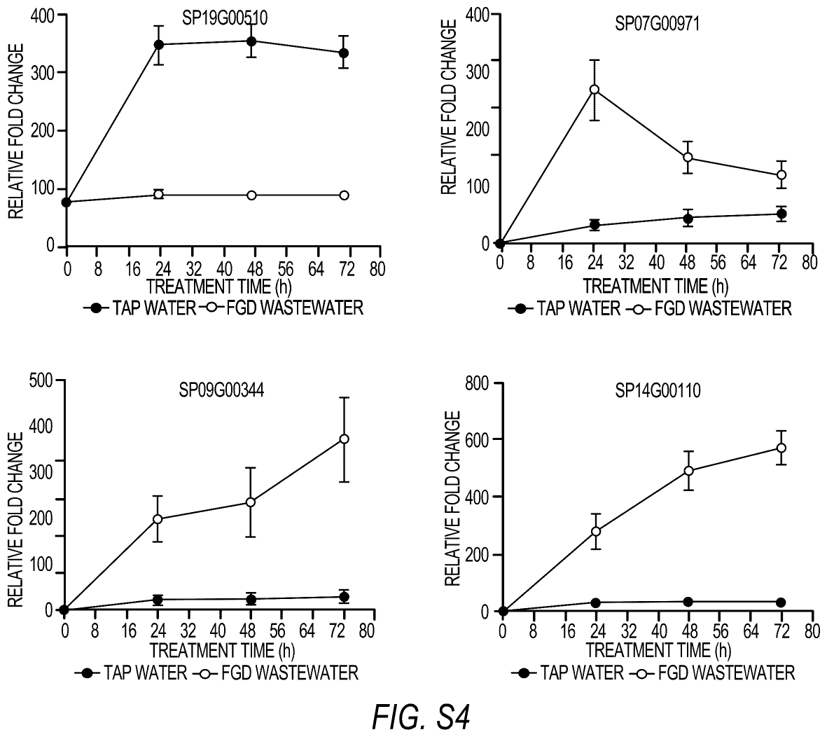

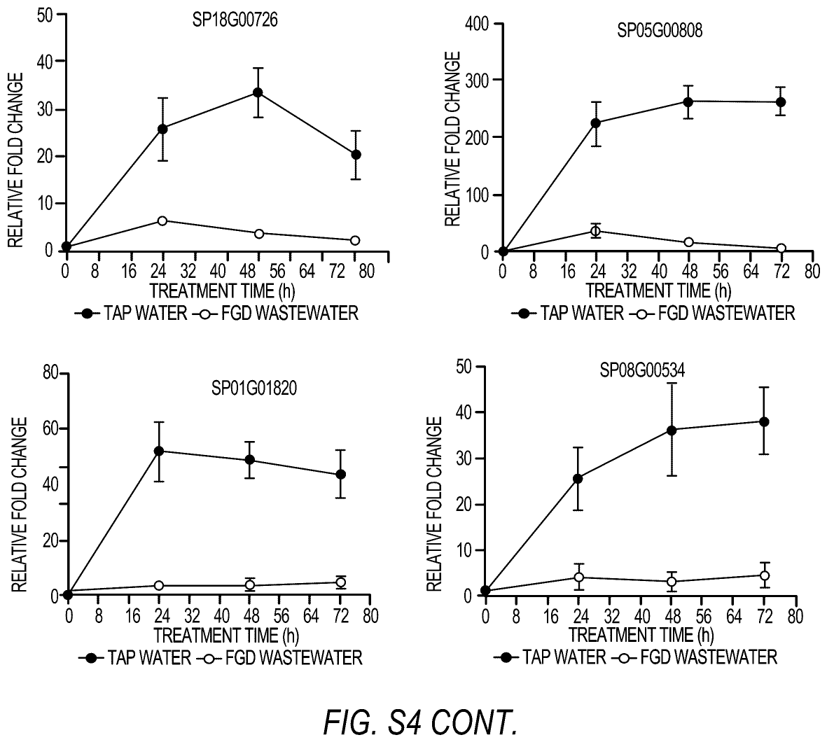

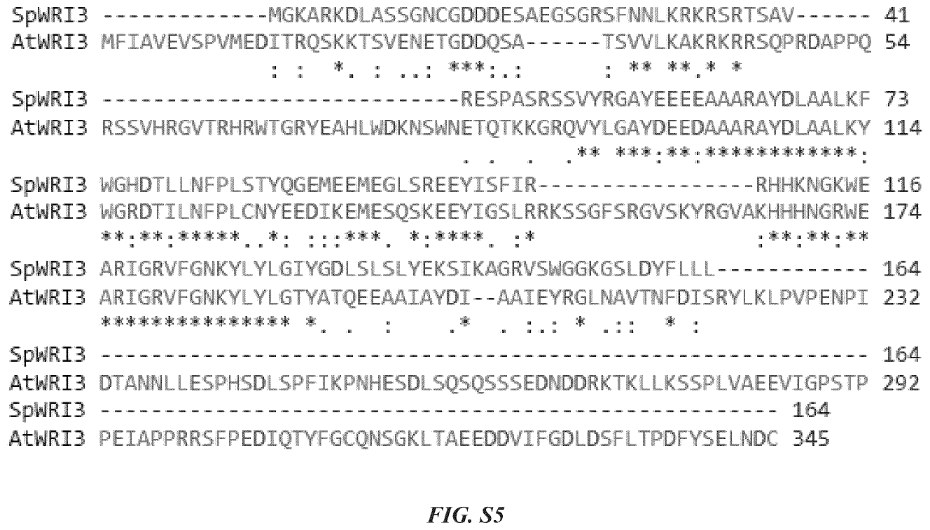

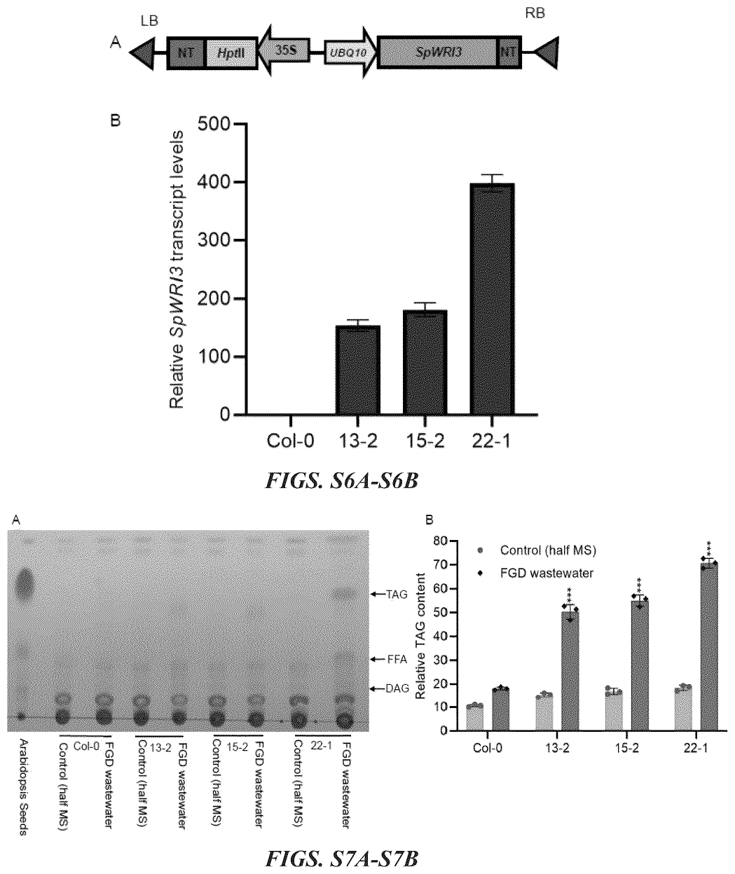

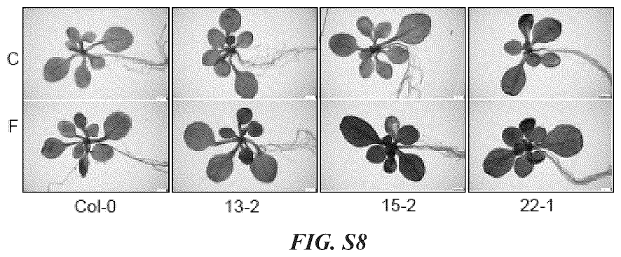

In plants and microalgae, various abiotic stresses induce the accumulation of different classes of lipases. These lipases can hydrolyze many substrates, including glycerolipids, to recycle head groups from the three different positions of a glycerol backbone to rapidly adjust the biophysical properties of membranes, thereby helping the plant cope with environmental stress (7). Our transcriptome analysis revealed the upregulation of the key lipase gene PLASTID LIPASE2 (PLIP2; Sp01g01058) in FGD wastewater-treated duckweed plantlets (Supplementary Table S11 (which is incorporated herein by reference from Muthan et al., J of Hazardous Materials 469 (2024) 133951 which can be found in the online version at doi:10.1016/j.jhazmat.2024.133951) and FIG. 3). In Arabidopsis, PLIP2 (encoded by At1g02660) is the plastid-localized lipid-degrading enzyme responsible for the degradation of MGDG that releases free fatty acids to produce oxylipins or jasmonic acid (89). Therefore, in S. polyrhiza, PLIP2 can mediate the hydrolysis of MGDG 36:6 (18:3/18:3) derived from the eukaryotic pathway and release 18:3 free fatty acids in response to FGD wastewater-induced heavy-metal stress. Subsequently, 18:3 free fatty acids are converted into TAG, oxylipins, or jasmonic acid to prevent cellular lipotoxicity (FIG. 12 A). Supporting this biochemical model, transcriptomic analysis revealed the upregulation of genes encoding a lipoxygenase (LOX; Sp03g00715) and oxo-phytodienoic acid reductases (Sp04g00566 and Sp18g00127) involved in the biosynthesis of oxylipins or jasmonic acid, respectively. These observations are consistent with previous reports suggesting a crucial role for PLIP2 in stress-induced membrane remodeling (7, 89).

### Altered TAG Accumulation

Acclimation to dynamically changing environmental conditions (such as abiotic stress) is critical for plant growth, development, and survival. One essential stage of acclimation to adverse conditions involves membrane lipid remodeling and the sequestration of toxic lipid intermediates from damaged membranes in TAGs. Our lipidomics and radiolabeling studies in S. polyrhiza suggested that under tap water and FGD wastewater conditions, the degraded products of polar lipids (mainly 18:3) are incorporated into TAG. In agreement with our data, several studies have shown that TAG accumulation in response to abiotic stress and the need for TAGs to support plant growth and development upon stress removal are common among vascular plants and microalgae (17, 28, 90, 29, 30).

We detected the considerable upregulation of SpWRI3 (Sp11g00856) in FGD wastewater-treated plantlets. Gene expression studies in the Arabidopsis wri1-1 mutant and transgenic plants overexpressing WRI1 revealed that WRI1 is a positive regulator of the fatty acid biosynthesis pathway (91-93). In Arabidopsis, WRI3 transcriptionally activated several fatty acid biosynthetic genes (94). We propose that SpWRI3 is likely involved in promoting the biosynthesis of pyruvate that is shunted to fatty acid and TAG biosynthesis in vegetative tissues of S. polyrhiza by activating downstream target genes such as genes encoding pyruvate dehydrogenase (PDH), acetyl-CoA carboxylase (ACCase), and caleosin (Supplementary Table S10, S11, which are incorporated herein by reference from Muthan et al., J of Hazardous Materials 469 (2024) 133951 which can be found in the online version at doi:10.1016/j.jhazmat.2024.133951). Thus, under FGD wastewater-induced heavy-metal stress, in addition to membrane remodeling involving altered TAG levels, SpWRI3 and caleosin may play a role in TAG biosynthesis and the mobilization of energy for cellular activities. In support of our hypothesis, recent studies in Arabidopsis suggested that caleosins have overlapping functions in TAG biosynthesis (95). In a recent study, exogenously expressing a sesame oleosin gene alone or in combination with Arabidopsis WRINKLED1 (AtWRI1) and the mouse diacylglycerol:acyl-CoA acytransferase2 (MmDGAT) gene under the control of an inducible promoter increased TAG production in Lemna japonica (49).

A Decline in Galactolipid and Phospholipid Biosynthesis in S. polyrhiza

We reasoned that treatment with tap water or FGD wastewater limited photosynthesis and the de novo fatty acid biosynthesis in S. polyrhiza due to the nutrient-limited conditions in tap water and heavy-metal stress from FGD wastewater. In support of this hypothesis, we noted the downregulation of several genes encoding key enzymes involved in synthesizing galactolipids and phospholipids in FGD wastewater-treated plantlets (Supplementary Tables S12, S13, which are incorporated herein by reference from Muthan et al., J of Hazardous Materials 469 (2024) 133951 which can be found in the online version at doi:10.1016/j.jhazmat.2024.133951). The decrease in the total amount of galactolipids and the change in their composition point to the degradation of DGDG and MGDG or inhibition of their biosynthesis. Consequently, the redistribution of lipid molecular species originating from the ER and chloroplast might alter membrane permeability and fluidity during heavy-metal stress in S. polyrhiza. In a previous study on duckweed, exposure of the mother colony to seven different heavy metals induced the premature release of daughter fronds, possibly due to changes in morphology or membrane composition (96). Similar to our observations, leaves and roots of cadmium-stressed almond (Prunus dulcis) seedlings exhibited substantial decreases in DGDG and MGDG levels (97). MGDG levels decreased considerably in sorghum (Sorghum bicolor) under salt stress, leading to a reduced ratio of plastidic to non-plastidic lipids (98). Sulfur starvation in lettuce (Lactuca sativa) led to changes in galactolipid levels (99).

In this study, the accumulation of PA was correlated with declines in phosphatidylcholine (PC) content. This observation points to a role for phospholipase D (PLD), which hydrolyzes phospholipids into PA and the head group choline, or for diacylglycerol kinase (DGK), which forms PA from DAG. Because PA plays a key role in regulating signal transduction and accumulates rapidly under different environmental stress conditions, PA might undergo dynamic changes that regulate the target proteins through binding under FGD wastewater-induced heavy-metal stress. Furthermore, PA signaling complexes are involved in regulating lipid metabolism and membrane properties and altering the growth of S. polyrhiza. This mechanism is common in plants exposed to freezing, drought, salinity, and hypoxia stresses (100-102). Further research is warranted to explore the effects of an increased PA pool size on membrane remodeling under heavy-metal stress.

Incorporation of Degraded Products of MGDG into TAG

DGDG and MGDG are major membrane components of chloroplasts in plants. These lipids are synthesized via the prokaryotic pathway in chloroplasts and via the eukaryotic pathway in the endoplasmic reticulum (25). One critical event in plants under abiotic stress is the degradation of membrane lipids (DGDG and MGDG), resulting in the production of unsaturated free fatty acids (UFAs), such as oleic (18:1), linoleic (18:2) and alpha-linolenic (18:3) acid (1, 22). The accumulation of these degraded membrane products, such as UFAs, is toxic to cells and causes oxidative stress and even cell death (23-25). To counteract these toxic effects, plants have evolved a sophisticated mechanism to remove polyunsaturated acyl groups from MGDG by enzymatic processes or other mechanisms that facilitate the incorporation of UAFs into triacylglycerols (TAGs) (28, 30). Chloroplast-localized HEAT INDUCIBLE LIPASE1 (HIL1) was shown to be responsible for the release of 18:3 polyunsaturated fatty acids from MGDG in Arabidopsis chloroplasts under heat stress. Lipidomics analysis pointed to the channeling of 18:3-CoA into PC and the subsequent sequestration of UFAs into TAG by phospholipid:diacylglycerol acyltransferase (PDAT) (103, 104). In a recent study, overexpressing the ADIPOSE TRIGLYCERIDE LIPASE-LIKE gene (ATGLL) significantly reduced the biosynthesis of MGDG and increased the production of TAG enriched with C18 in transgenic Arabidopsis leaves. However, the disruption of ATGLL via RNAi resulted in a decrease in TAG accumulation (105). Based on our lipidomics and radiolabeling data, the accumulation of TAG under heavy-metal stress induced by FGD wastewater might play a significant role in the adaptation of S. polyrhiza by sequestering the toxic degradation products of MGDG. Further research is needed to unravel the metabolic pathway and enzymes involved in directing UAFs from MGDG to TAG in S. polyrhiza treated with FGD wastewater.

### Increased TAG Degradation and β-Oxidation

In this study, FGD wastewater treatment led to the upregulation of genes encoding several lipases potentially involved in TAG degradation, such as triacylglycerol lipase, monoacylglycerol lipase, and patatin-like acyl-hydrolase (Supplementary Tables S10, S11, which are incorporated herein by reference from Muthan et al., J of Hazardous Materials 469 (2024) 133951 which can be found in the online version at doi:10.1016/j.jhazmat.2024.133951). In S. polyrhiza, heavy-metal stress may induce the accumulation of lipases that initiate the degradation of TAGs to sustain a carbon supply for cellular metabolism. During this response, triacylglycerol lipases (SUGAR-DEPENDENT1 (SDP1) and TAG lipases (TAGLs)) are presumably involved in TAG degradation, leading to the release of DAGs and MAGs. Furthermore, monoacylglycerol lipases (four MAGLs) convert monoacylglycerols (MAGs) to free fatty acids and glycerol; the free fatty acids subsequently undergo β-oxidation in peroxisomes. In support of our observation, in Arabidopsis, several TAG lipases were identified in germinating seedlings, such as SDP1, which is responsible for mobilizing TAGs from lipid droplets during seed germination (106). Knocking down orthologs of Arabidopsis SDP1 in soybean (Glycine max) and Physaria fendleri increased seed oil biosynthesis (107-109).

Based on previous and current data, we suggest that heavy-metal stress induced by FGD wastewater triggers the degradation of membrane lipids in S. polyrhiza due to the actions of various lipases. The release of lipid degradation products, in turn, activates the β-oxidation pathway to generate acetyl-CoA (for synthesizing new membranes) and other signaling molecules (such as ROS and jasmonic acid) to enhance survival under dynamic environmental stress conditions. In support of our hypothesis, in FGD wastewater-treated S. polyrhiza, DEG analysis revealed the upregulation of all genes encoding key enzymes involved in the β-oxidation pathway (Supplementary Tables S10, S11). Consistent with our observation, low-oxygen stress drastically altered fatty acid degradation via β-oxidation in different Brassicaceae plants under low-oxygen or submergence stress (110). Overexpressing the acyl-coenzyme A oxidase 3 (ACXe) gene, which is critical for β-oxidation, increased tolerance to drought and salt stress in cotton (Gossypium hirsutum) (111). Cold stress in rice (Oryza sativa) led to the upregulation of several genes involved in fatty acid β-oxidation, thereby increasing cold tolerance (112). Overexpressing the cytochrome b5 reductase 1 (CBR1) gene enhanced osmatic-stress tolerance in Arabidopsis by enhancing the β-oxidation of polyunsaturated fatty acids (113).

### Altered Surface Lipid Biosynthesis

The aerial surface parts of plants are covered with a cuticle comprising cutin and waxes that protect plants from stomatal water loss, drought, radiation exposure, and pathogen attack (114-116, 87, 117). FGD wastewater stress appeared to reduce total wax accumulation at 48 h into treatment. However, this difference was not statistically significant. FGD wastewater stress did, however, affect the accumulation of several wax constituents. By 72 h of treatment, 18:0, 20:0, 22:0, and 24:0 FFAs accumulated to higher levels under FGD wastewater than tap water treatment. This accumulation was accompanied by a concomitant decrease in the levels of very-long-chain fatty acyl-containing sn-1(3) MAGs at both 48 and 72 h, which may be related to the observed increase in MAG lipase gene (Sp06g01222 and Sp03g01202) transcript abundance in FGD wastewater-treated S. polyrhiza plantlets (Supplementary Table S10). We observed relatively large increases in the accumulation of 24:0 and 26:0 fatty primary alcohols (1-alcohols) at 24, 48, and 72 h into FGD treatment. However, these increases were not related to any changes in cuticular lipid gene-related transcript abundance (i.e., fatty acyl reductase genes) apart from an elevated abundance of transcript for the gene encoding long-chain acyl-CoA synthetase 2 (LACS2, Sp03g00142) at 24 h into FGD wastewater treatment (Supplementary Table S11).

In terrestrial plants, the cuticle is often regarded as an adaptation to help prevent water loss. However, in aquatic plants, the role of the cuticle is likely reversed, with cuticular waxes restricting water entry into leaves to prevent waterlogging and associated hypoxia, as observed in sacred lotus (Nelumbo nucifera) (118, 119). The triterpenoid-rich waxes of S. polyrhiza might be important for repelling water, as observed for the aerenchymatous phellem of soybean, whose triterpenoid-rich waxes accumulate in response to waterlogging to better maintain air spaces and prevent oxygen loss under water (120). Furthermore, although it is well known that cuticular wax deposition increases in vascular plants in response to abiotic stress and stimuli such as the phytohormone abscisic acid (ABA) (121, 122, 116), aquatic plants or lower plants (e.g., bryophytes) that typically grow in wet conditions likely have different responses with respect to cuticular wax accumulation under stress compared to terrestrial plants. In agreement with this notion, in the moss Physcomitrium patens, the expression of cuticle-related genes was suppressed in response to exogenous ABA treatment (123). We suggest that unlike terrestrial plants, cuticular wax likely plays less or no role in FGD wastewater-induced heavy metal-stress tolerance in the aquatic plant S. polyrhiza.

### Regulation of Starch Biosynthesis During FGD Wastewater-Induced Heavy-Metal Stress

Our biochemical data show that heavy-metal stress induced by FGD wastewater resulted in the increased accumulation of carbohydrates; the increased starch grain size (suggesting a large proportion of built-up starch in the chloroplast) might be due to the decreased demand for cellular metabolism due to inhibited plant growth. In agreement with this idea, S. polyrhiza also accumulated starch from day 3 to 5 in response to salt stress (45). Furthermore, several studies have reported an increase in starch and soluble sugar accumulation in response to salt stress, cold stress, and increased CO2 and ozone concentrations (124-128).

In this study, several genes encoding starch biosynthesis or degradation enzymes were upregulated at 24 to 72 h into FGD wastewater treatment (Supplementary Table S14, which is incorporated herein by reference from Muthan et al., J of Hazardous Materials 469 (2024) 133951 which can be found in the online version at doi:10.1016/j.jhazmat.2024.133951). ADG-Glc PPase is a key enzyme required for catalysis of the committed step of starch biosynthesis in plants (129). Analysis of Arabidopsis mutants revealed that α-GLUCAN WATER DIKINASE (GWD), BETA-AMYLASE1 (BAM1), ALPHA-AMYLASE-LIKE3 (AMY3), and ALPHA-GLUCAN PHOSPHORYLASE1 (PHS1) are major enzymes required for starch degradation under water deficit, cold, or salt stress conditions (130, 131). In Arabidopsis, phytochrome-interacting bHLH transcription factors help prevent starch degradation by suppressing QUA-QUINE STARCH (QQS) expression (132). These findings suggest that FGD wastewater treatment triggered photosynthesis and starch biosynthesis in S. polyrhiza at the early stages (24 h) of the experiment. However, at later stages of FGD wastewater exposure (72 h), photosynthesis and starch biosynthesis were inhibited, with a concomitant increase in expression of genes encoding starch degradation pathway enzymes and genes in glucose/sugar signaling pathways. This biochemical switch presumably helps provide carbon flux to support cellular metabolism under stress conditions (FIG. 12B).

### Arabidopsis Plants Expressing SpWRI3 Show Increased Tolerance to FGD Wastewater

Several transcription factors are implicated in responses to biotic and abiotic stress and associated signal transduction in plants (133-135). The most commonly studied transcription factors in response to heavy metals are basic leucine zipper (bZIP), basic helix-loop-helix (bHLH), and WRKY transcription factors (136, 137, 135). Members of the AP2/ERF transcription factor family are unique to plants and play critical roles in plant development and environmental-stress tolerance (138). Additionally, many AP2/ERF transcription factors mediate ABA and ethylene responses (139). In this study, using Arabidopsis, we functionally characterized SpWRI3, a member of the AP2/ERF transcription factor family, and demonstrated its role in increased GSH production to provide tolerance to heavy-metal stress induced by FGD wastewater. Our results agree with the finding that the heterologous expression of the pepper (Capsicum annuum) AP2/ERF transcription factor gene CaPF1 (Pathogen and freezing tolerance-related protein 1) in transgenic Virginia pine (Pinus virginiana) enhanced tolerance to heavy metals (Tang et al., 2005). Similarly, ectopic expression of CaPF1 in potato (Solanum tuberosum) increased tolerance to heavy metals and other stresses (140). Exogenously expressing the durum wheat (Triticum durum) transcription factor gene TdSHN1 conferred tolerance to multiple heavy metals in tobacco (141). Exogenously expressing the soybean (Glycine max) Ap2/ERF transcription factor gene GmABR1 increased tolerance to aluminum stress in transgenic Arabidopsis (63). Furthermore, the strong upregulation of SpWRI3 in response to FGD wastewater treatment in duckweed suggests that SpWRI3 might be required for tolerance to heavy-metal stress induced by FGD wastewater. Based on our findings, we propose that the ectopic expression of SpWRI3 in Arabidopsis enhanced tolerance to FGD wastewater by increasing the production of GSH and decreasing the production of H2O2, lipid peroxidation (based on MDA levels), and superoxide radicals, presumably by upregulating heavy metal stress-related genes or transcription factor genes involved in ABA biosynthesis and/or GSH pathways. In support of our observation, Arabidopsis WRI3 plays a role in ABA-mediated tolerance to water deprivation stress (88). A yeast one-hybrid assay showed that several AP2/ERF family transcription factors bind to cis-elements present in the promoter of Arabidopsis STRESS-ASSOCIATED PROTEIN 13 (SAP13) and enhance tolerance to heavy metals and other stresses (142).

### CONCLUSION

Here we describe the changes in the metabolome and expression of lipid and carbohydrate pathway genes in S. polyrhiza in response to treatment with FGD wastewater containing heavy metals. FGD wastewater treatment modulated glycerolipid and phospholipid biosynthesis and composition and increased the accumulation of TAG enriched in 18:3 unsaturated fatty acids. We propose a working model describing how the incorporation of degraded products of the major membrane lipid monogalactosyldiacylglycerol (MGDG) 36:6 (18:3/18:3) into TAGs likely prevents cellular lipotoxicity caused by the presence of unsaturated free fatty acids (FIG. 12 A, B). We discovered a novel AP2/ERF transcription factor gene SpWRI3 (Sp11g00856), one of the most highly upregulated genes in S. polyrhiza in response to FGD wastewater treatment. The ectopic expression of SpWRI3 enhanced tolerance to FGD wastewater treatment in transgenic Arabidopsis. SpWRI3 might be a novel candidate gene to develop heavy metal-tolerant crops. Taken together, we provided physiological, biochemical, lipidomic, and transcriptomic evidence to confirm the FGD wastewater-induced heavy metal stress-tolerance mechanism in S. polyrhiza.

### Environmental Implications

Flue gas desulfurization (FGD) is a commercially established procedure in coal-fired power plants that utilizes highly oxygenated limestone slurries to convert sulfur gas to a soluble form in scrubbing water. This method is primarily used to improve air quality by removing sulfur dioxide from flue gas emissions. FGD blowdown wastewater is known to contain trace concentrations of heavy metals. The removal of heavy metals in FGD wastewater in thermoelectric power plants presents an industrial challenge due to the large volumes produced. Because heavy metals are freely soluble in water and easily taken up by plants, and subsequent in their entry into the food web, is a source of human heavy-metal intake via biomagnification. Thus, a genetic understanding of how plants respond to heavy metals in FGD wastewater and developing FGD wastewater tolerant plants through genetic improvement is a sustainable measure to address heavy-metal contamination in FGD wastewater. In this study, the physiological, biochemical, anatomical, lipidomics, genomics, and genetic response of duckweed (Spirodela polyrhiza) to FGD wastewater-induced heavy metals were systematically analyzed to reveal the heavy metal-tolerance mechanism. These results provide a basis for safe crop production in heavy metal-contaminated soil or water.

### BIBLIOGRAPHY

[1] Angulo-Bejarano, P. I., Puente-Rivera, J., Cruz-Ortega, R., 2021. Metal and metalloid toxicity in plants: an overview on molecular aspects. Plants 10 (4), 635.
[2] Dutta, S., Mitra, M., Agarwal, P., Mahapatra, K., De, S., Sett, U., et al., 2018. Oxidative and genotoxic damages in plants in response to heavy metal stress and maintenance of genome stability. Plant Signal Behav 13 (8), e1460048.
[3] Berni, R., Luyckx, M., Xu, X., Legay, S., Sergeant, K., Hausman, J.-F., et al., 2019. Reactive oxygen species and heavy metal stress in plants: Impact on the cell wall and secondary metabolism. Environ Exp Bot 161, 98-106.
[4] Guerra Sierra, B. E., Mu\tilde{ }noz Guerrero, J., Sokolski, S., 2021. Phytoremediation of heavy metals in tropical soils an overview. Sustainability 13 (5), 2574.
[5] Kumar, S., Prasad, S., Yadav, K. K., Shrivastava, M., Gupta, N., Nagar, S., et al., 2019. Hazardous heavy metals contamination of vegetables and food chain: Role of sustainable remediation approaches—a review. Environ Res 179, 108792.
[6] Yan, A., Wang, Y., Tan, S. N., Mohd Yusof, M. L., Ghosh, S., Chen, Z., 2020. Phytoremediation: a promising approach for revegetation of heavy metal-polluted land. Front Plant Sci 11, 359.
[7] Cook, R., Lupette, J., Benning, C., 2021. The role of chloroplast membrane lipid metabolism in plant environmental responses. Cells 10 (3), 706.
[8] Prasad, M., Hagemeyer, J., Devi, S. R., Prasad, M., 1999. Membrane lipid alterations in heavy metal exposed plants. Heavy Met Stress Plant: Mol Ecosyst 99-116.
[9] Barrero-Sicilia, C., Silvestre, S., Haslam, R. P., Michaelson, L. V., 2017. Lipid remodelling: Unravelling the response to cold stress in Arabidopsis and its extremophile relative Eutrema salsugineum. Plant Sci 263, 194-200.
[10] Wang, Y., Zhang, X., Huang, G., Feng, F., Liu, X., Guo, R., et al., 2020. Dynamic changes in membrane lipid composition of leaves of winter wheat seedlings in response to PEG-induced water stress. BMC Plant Biol 20 (1), 1-15.
[11] Chaffai, R., Cherif, A., 2020. The cadmium-induced changes in the polar and neutral lipid compositions suggest the involvement of triacylglycerol in the defense response in maize. Physiol Mol Biol Plants 26 (1), 15-23.
[12] Ghori, N.-H., Ghori, T., Hayat, M., Imadi, S., Gul, A., Altay, V., et al., 2019. Heavy metal stress and responses in plants. Int J Environ Sci Technol 16, 1807-1828.
[13] Wagatsuma, T., 2017. The membrane lipid bilayer as a regulated barrier to cope with detrimental ionic conditions: making new tolerant plant lines with altered membrane lipid bilayer. Soil Sci Plant Nutr 63 (5), 507-516.
[14] Spicher, L., Glauser, G., Kessler, F., 2016. Lipid antioxidant and galactolipid remodeling under temperature stress in tomato plants. Front Plant Sci 7, 167.
[15] P\acute{ }al, M., Lesk\acute{ }o, K., Janda, T., P\acute{ }aldi, E., Szalai, G., 2007. Cadmium-induced changes in the membrane lipid composition of maize plants. Cereal Res Commun 35 (4), 1631-1642.
[16] Nesterov, V., Rozentsvet, O., Murzaeva, S., 2009. Changes in lipid composition in the tissues of fresh-water plant Hydrilla verticillata induced by accumulation and elimination of heavy metals. Russ J Plant Physiol 56, 85-93.
[17] Higashi, Y., Okazaki, Y., Myouga, F., Shinozaki, K., Saito, K., 2015. Landscape of the lipidome and transcriptome under heat stress in Arabidopsis thaliana. Sci Rep 5 (1), 10533.
[18] Pfaff, J., Denton, A. K., Usadel, B., Pfaff, C., 2020. Phosphate starvation causes different stress responses in the lipid metabolism of tomato leaves and roots. Biochim Et Biophys Acta (BBA)-Mol Cell Biol Lipids 1865 (9), 158763.
[19] Zhang, J.-T., Zhu, J.-Q., Zhu, Q., Liu, H., Gao, X.-S., Zhang, H.-X., 2009. Fatty acid desaturase-6 (Fad6) is required for salt tolerance in Arabidopsis thaliana. Biochem Biophys Res Commun 390 (3), 469-474.
[20] Ashraf, U., Tang, X., 2017. Yield and quality responses, plant metabolism and metal distribution pattern in aromatic rice under lead (Pb) toxicity. Chemosphere 176, 141-155.
[21] Fujita, M., Hasanuzzaman, M., 2022. Approaches to enhancing antioxidant defense in plants. MDPI, p. 925.
[22] He, M., Ding, N.-Z., 2020. Plant unsaturated fatty acids: multiple roles in stress response. Front Plant Sci 11, 562785.
[23] Olzmann, J. A., Carvalho, P., 2019. Dynamics and functions of lipid droplets. Nat Rev Mol Cell Biol 20 (3), 137-155.
[24] Shimada, K., Reznik, E., Stokes, M. E., Krishnamoorthy, L., Bos, P. H., Song, Y., et al., 2018. Copper-binding small molecule induces oxidative stress and cell-cycle arrest in glioblastoma-patient-derived cells. Cell Chem Biol 25 (5), 585-594 e587.
[25] Xu, C., Fan, J., Shanklin, J., 2020. Metabolic and functional connections between cytoplasmic and chloroplast triacylglycerol storage. Prog Lipid Res 80, 101069.
[26] Jayasri, M., Suthindhiran, K., 2017. Effect of zinc and lead on the physiological and biochemical properties of aquatic plant Lemna minor: its potential role in phytoremediation. Appl Water Sci 7, 1247-1253.
[27] Lu, J., Xu, Y., Wang, J., Singer, S. D., Chen, G., 2020. The role of triacylglycerol in plant stress response. Plants 9 (4), 472.
[28] Moellering, E. R., Muthan, B., Benning, C., 2010. Freezing tolerance in plants requires lipid remodeling at the outer chloroplast membrane. Science 330 (6001), 226-228.
[29] Xu, L., Pan, R., Zhang, W., 2020. Membrane lipids are involved in plant response to oxygen deprivation. Plant Signal Behav 15 (7), 1771938.
[30] Yu, L., Zhou, C., Fan, J., Shanklin, J., Xu, C., 2021. Mechanisms and functions of membrane lipid remodeling in plants. Plant J 107 (1), 37-53.
[31] AL-Janabi, Q. A., Al-Kalidy, S. K. A., Hameed, Z. B., 2021. Effects of heavy metals on physiological status for Schoenoplectus litoralis & Salvinia natans L. IOP Conference Series: Earth and Environmental Science. IOP Publishing, 012012.
[32] Bai, L., Liu, X.-L., Hu, J., Li, J., Wang, Z.-L., Han, G., et al., 2018. Heavy metal accumulation in common aquatic plants in rivers and lakes in the Taihu Basin. Int J Environ Res Public Health 15 (12), 2857.
[33] Finnegan, P., Chen, W., 2012. Arsenic toxicity: the effects on plant metabolism. Front Physiol 3, 182.
[34] Uddin, M. M., Zakeel, M. C. M., Zavahir, J. S., Marikar, F. M., Jahan, I., 2021. Heavy metal accumulation in rice and aquatic plants used as human food: A general review. Toxics 9 (12), 360.
[35] Xing, W., Huang, W., Liu, G., 2010. Effect of excess iron and copper on physiology of aquatic plant Spirodela polyrrhiza (L.) Schleid. Environ Toxicol: Int J 25 (2), 103-112.
[36] Rofkar, J. R., Dwyer, D. F., Bobak, D. M., 2014. Uptake and toxicity of arsenic, copper, and silicon in Azolla caroliniana and Lemna minor. Int J Phytoremediat 16 (2), 155-166.
[37] Huang, S., Song, Q., Li, Q., Zhang, H., Luo, X., Zheng, Z., 2020. Damage of heavy metals to Vallisneria natans (V. natans) and characterization of microbial community in biofilm. Aquat Toxicol 225, 105515.
[38] Teisseire, H., Vernet, G., 2000. Ascorbate and glutathione contents in duckweed, Lemna minor, as biomarkers of the stress generated by copper, folpet and diuron. Biomarkers 5 (4), 263-273.
[39] Bog, M., Appenroth, K.-J., Sree, K. S., 2019. Duckweed (Lemnaceae): its molecular taxonomy. Front Sustain Food Syst 3, 117.
[40] Fourounjian, P., Tang, J., Tanyolac, B., Feng, Y., Gelfand, B., Kakrana, A., et al., 2019. Post-transcriptional adaptation of the aquatic plant Spirodela polyrhiza under stress and hormonal stimuli. Plant J 98 (6), 1120-1133.
[41] Yang, G.-L., Feng, D., Liu, Y.-T., Lv, S.-M., Zheng, M.-M., Tan, A.-J., 2021. Research progress of a potential bioreactor: Duckweed. Biomolecules 11 (1), 93.
[42] An, D., Wang, W., 2020. The journey of spirodela whole-genome sequencing. Duckweed Genomes 77-85.
[43] Khellaf, N., Zerdaoui, M., 2009. Growth response of the duckweed Lemna minor to heavy metal pollution. J Environ Health Sci Eng 6 (3), 161-166.
[44] Wang, W., Haberer, G., Gundlach, H., Gl\ddot{ }aßer, C., Nussbaumer, T., Luo, M., et al., 2014. The Spirodela polyrhiza genome reveals insights into its neotenous reduction fast growth and aquatic lifestyle. Nat Commun 5, 3311.
[45] Fu, L., Ding, Z., Sun, X., Zhang, J., 2019. Physiological and transcriptomic analysis reveals distorted ion homeostasis and responses in the freshwater plant Spirodela polyrhiza L. under salt stress. Genes 10 (10), 743.
[46] Hoang, P. N., Michael, T. P., Gilbert, S., Chu, P., Motley, S. T., Appenroth, K. J., et al., 2018. Generating a high-confidence reference genome map of the Greater Duckweed by integration of cytogenomic, optical mapping, and Oxford Nanopore technologies. Plant J 96 (3), 670-684.
[47] Xu, H., Yu, C., Xia, X., Li, M., Li, H., Wang, Y., et al., 2018. Comparative transcriptome analysis of duckweed (Landoltia punctata) in response to cadmium provides insights into molecular mechanisms underlying hyperaccumulation. Chemosphere 190, 154-165.
[48] Yan, Y., Candreva, J., Shi, H., Ernst, E., Martienssen, R., Schwender, J., et al., 2013. Survey of the total fatty acid and triacylglycerol composition and content of 30 duckweed species and cloning of a Δ6-desaturase responsible for the production of γ-linolenic and stearidonic acids in Lemna gibba. BMC Plant Biol 13 (1), 1-14.
[49] Liang, Y., Yu, X. H., Anaokar, S., Shi, H., Dahl, W. B., Cai, Y., et al., 2023. Engineering triacylglycerol accumulation in duckweed (Lemna japonica). Plant Biotechnol J 21 (2), 317-330.
[50] Abdullah, H. M., Chhikara, S., Akbari, P., Schnell, D. J., Pareek, A., Dhankher, O. P., 2018. Comparative transcriptome and metabolome analysis suggests bottlenecks that limit seed and oil yields in transgenic Camelina sativa expressing diacylglycerol acyltransferase 1 and glycerol-3-phosphate dehydrogenase. Biotechnol Biofuels 11, 1-28.
[51] Gupta, A., Senthil-Kumar, M., 2017. Transcriptome changes in Arabidopsis thaliana infected with Pseudomonas syringae during drought recovery. Sci Rep 7 (1), 9124.
[52] Le Nguyen, K., Grondin, A., Courtois, B., Gantet, P., 2019. Next-generation sequencing accelerates crop gene discovery. Trends Plant Sci 24 (3), 263-274.
[53] Xiao, M., Zhang, Y., Chen, X., Lee, E.-J., Barber, C. J., Chakrabarty, R., et al., 2013. Transcriptome analysis based on next-generation sequencing of non-model plants producing specialized metabolites of biotechnological interest. J Biotechnol 166 (3), 122-134.
[54] Aulakh, K., Durrett, T. P., 2019. The plastid lipase PLIP1 is critical for seed viability in diacylglycerol acyltransferase1 mutant seed. Plant Physiol 180 (4), 1962-1974.
[55] Cao, D., Liu, Y., Ma, L., Jin, X., Guo, G., Tan, R., et al., 2018. Transcriptome analysis of differentially expressed genes involved in selenium accumulation in tea plant (Camellia sinensis). PLoS One 13 (6), e0197506.
[56] Chen, Y., Zhi, J., Zhang, H., Li, J., Zhao, Q., Xu, J., 2017. Transcriptome analysis of Phytolacca americana L. in response to cadmium stress. PLoS One 12 (9), e0184681.
[57] Cruz, Y., Villar, S., Guti\acute{ }errez, K., Montoya-Ruiz, C., Gallego, J. L., Delgado, Md.P., et al., 2021. Gene expression and morphological responses of Lolium perenne L. exposed to cadmium (Cd2+) and mercury (Hg2+). Sci Rep 11 (1), 1-11.
[58] Cheng, P., Zhou, C., Wang, Y., Xu, Z., Xu, J., Zhou, D., et al., 2018. Comparative transcriptome analyses of oleaginous Botryococcus braunii race A reveal significant differences in gene expression upon cobalt enrichment. Biotechnol Biofuels 11 (1), 1-19.
[59] El-Sappah, A. H., Elbaiomy, R. G., Elrys, A. S., Wang, Y., Zhu, Y., Huang, Q., et al., 2021. Genome-wide identification and expression analysis of metal tolerance protein gene family in Medicago truncatula under a broad range of heavy metal stress. Front Genet 1340.
[60] Sharaf, A., De Michele, R., Sharma, A., Fakhari, S., Oborník, M., 2019. Transcriptomic analysis reveals the roles of detoxification systems in response to mercury in Chromera velia. Biomolecules 9 (11), 647.
[61] An, D., Li, C., Zhou, Y., Wu, Y., Wang, W., 2018. Genomes and transcriptomes of duckweeds. Front Chem 6, 230.
[62] Fu, L., Ding, Z., Tan, D., Han, B., Sun, X., Zhang, J., 2020. Genome-wide discovery and functional prediction of salt-responsive lncRNAs in duckweed. BMC Genom 21 (1), 14.
[63] Wang, Q., Wilfong, W., Kail, B., Ji, T., Shi, F., Gray, M., 2023. Effective removal of trace-level toxic metals from flue gas desulfurization wastewater using SiO2 supported hydrogel sorbent. Mater Today Sustain 21, 100324.
[64] Christie, W., 1991. Gas chromatographic analysis of fatty acid methyl esters with high precision. Lipid Technol 3, 97-98.
[65] Lee, J., Welti, R., Schapaugh, W. T., Trick, H. N., 2011. Phospholipid and triacylglycerol profiles modified by PLD suppression in soybean seed. Plant Biotechnol J 9 (3), 359-372.
[66] Fruehan, C., Johnson, D., Welti, R., 2018. LipidomeDB Data Calculation Environment has been updated to process direct-infusion multiple reaction monitoring data. Lipids 53 (11-12), 1019-1020.
[67] Zhou, Z., Marepally, S. R., Nune, D. S., Pallakollu, P., Ragan, G., Roth, M. R., et al., 2011. LipidomeDB Data Calculation Environment: Online processing of direct-infusion mass spectral data for lipid profiles. Lipids 46, 879-884.
[68] Shiva, S., Vu, H. S., Roth, M. R., Zhou, Z., Marepally, S. R., Nune, D. S., et al., 2013. Lipidomic analysis of plant membrane lipids by direct infusion tandem mass spectrometry. Plant Lipid Signal Protoc 79-91.
[69] Sanjaya, Miller, R., Durrett, T. P., Kosma, D. K., Lydic, T. A., Muthan, B., et al., 2013. Altered lipid composition and enhanced nutritional value of Arabidopsis leaves following introduction of an algal diacylglycerol acyltransferase 2. Plant Cell 25 (2), 677-693.
[70] Bolger, A. M., Lohse, M., Usadel, B., 2014. Trimmomatic: a flexible trimmer for Illumina sequence data. Bioinformatics 30 (15), 2114-2120.
[71] Andrews, S., 2010. FastQC: a quality control tool for high throughput sequence data. Babraham Bioinforma, Babraham Inst, Camb, U Kingd.
[72] Dobin, A., Davis, C. A., Schlesinger, F., Drenkow, J., Zaleski, C., Jha, S., et al., 2013. STAR: ultrafast universal RNA-seq aligner. Bioinformatics 29 (1), 15-21.
[73] Liao, Y., Smyth, G. K., Shi, W., 2013. The Subread aligner: fast, accurate and scalable read mapping by seed-and-vote. Nucleic Acids Res 41 (10) e108-e108.
[74] Law, C. W., Chen, Y., Shi, W., Smyth, G. K., 2014. voom: Precision weights unlock linear model analysis tools for RNA-seq read counts. Genome Biol 15 (2), 1-17.
[75] Fan, J. L., Yan, C. S., Xu, C. C., 2013. Phospholipid: diacylglycerol acyltransferase-mediated triacylglycerol biosynthesis is crucial for protection against fatty acid-induced cell death in growing tissues of Arabidopsis. Plant J 76 (6), 930-942.
[76] Clough, S. J., Bent, A. F., 1998. Floral dip: a simplified method for Agrobacterium-mediated transformation of Arabidopsis thaliana. Plant J 16 (6), 735-743.
[77] Lichtenthaler, H. K., Wellburn, A. R., 1983. Determinations of total carotenoids and chlorophylls a and b of leaf extracts in different solvents. Portland Press Ltd.
[78] Sanjaya, Durrett, T. P., Weise, S. E., Benning, C., 2011. Increasing the energy density of vegetative tissues by diverting carbon from starch to oil biosynthesis in transgenic Arabidopsis. Plant Biotechnol J 9 (8), 874-883.
[79] Santoro, N., Cantu, S. L., Tornqvist, C.-E., Falbel, T. G., Bolivar, J. L., Patterson, S. E., et al., 2010. A high-throughput platform for screening milligram quantities of plant biomass for lignocellulose digestibility. BioEnergy Res 3, 93-102.
[80] Grellet Bournonville, C. F., Diaz-Ricci, J. C., 2011. Quantitative determination of superoxide in plant leaves using a modified NBT staining method. Phytochem Anal 22 (3), 268-271.
[81] Orozco-C\acute{ }ardenas, M. L., Narv\acute{ }aez-V\acute{ }asquez, J., Ryan, C. A., 2001. Hydrogen peroxide acts as a second messenger for the induction of defense genes in tomato (1), 179-191.
[82] Heath, R. L., Packer, L., 1968. Photoperoxidation in isolated chloroplasts: I. Kinetics and stoichiometry of fatty acid peroxidation. Arch Biochem Biophys 125 (1), 189-198.
[83] Hodges, D. M., DeLong, J. M., Forney, C. F., Prange, R. K., 1999. Improving the thiobarbituric acid-reactive-substances assay for estimating lipid peroxidation in plant tissues containing anthocyanin and other interfering compounds. Planta 207, 604-611.
[84] EPA, U., 1994. Determination of metals and trace elements in water and wastes by inductively coupled plasma-atomic emission spectrometry. Environ Monit Syst Lab, Res.
[85] Barker, D. J., Mannucchi, G. A., Salvi, S. M., Stuckey, D. C., 1999. Characterisation of soluble residual chemical oxygen demand (COD) in anaerobic wastewater treatment effluents. Water Res 33 (11), 2499-2510.
[86] Borisjuk, N., Peterson, A. A., Lv, J., Qu, G., Luo, Q., Shi, L., et al., 2018. Structural and biochemical properties of duckweed surface cuticle. Front Chem 6, 317.
[87] Li-Beisson, Y., Shorrosh, B., Beisson, F., Andersson, M. X., Arondel, V., Bates, P. D., et al., 2013. Acyl-lipid metabolism. Arab Book/Am Soc Plant Biol 11.
[88] Lee, S.-j, Cho, D.-i, Kang, J.-y, Kim, S. Y., 2009. An ARIA-interacting AP2 domain protein is a novel component of ABA signaling. Mol Cells 27, 409-416.
[89] Wang, K., Guo, Q., Froehlich, J. E., Hersh, H. L., Zienkiewicz, A., Howe, G. A., et al., 2018. Two abscisic acid-responsive plastid lipase genes involved in jasmonic acid biosynthesis in Arabidopsis thaliana. Plant Cell 30 (5), 1006-1022.
[90] Tan, W.-J., Yang, Y.-C., Zhou, Y., Huang, L.-P., Xu, L., Chen, Q.-F., et al., 2018. DIACYLGLYCEROL ACYLTRANSFERASE and DIACYLGLYCEROL KINASE modulate triacylglycerol and phosphatidic acid production in the plant response to freezing stress. Plant Physiol 177 (3), 1303-1318.
[91] Baud, S., Wuilleme, S., To, A., Rochat, C., Lepiniec, L., 2009. Role of WRINKLED1 in the transcriptional regulation of glycolytic and fatty acid biosynthetic genes in Arabidopsis. Plant J 60 (6), 933-947.
[92] Maeo, K., Tokuda, T., Ayame, A., Mitsui, N., Kawai, T., Tsukagoshi, H., et al., 2009. An AP2-type transcription factor, WRINKLED1, of Arabidopsis thaliana binds to the AW-box sequence conserved among proximal upstream regions of genes involved in fatty acid synthesis. Plant J 60 (3), 476-487.
[93] Ruuska, S. A., Girke, T., Benning, C., Ohlrogge, J. B., 2002. Contrapuntal networks of gene expression during Arabidopsis seed filling. Plant Cell 14 (6), 1191-1206.
[94] To, A., Joub\grave{ }es, J., Barthole, G., L\acute{ }ecureuil, A., Scagnelli, A., Jasinski, S., et al., 2012. WRINKLED transcription factors orchestrate tissue-specific regulation of fatty acid biosynthesis in Arabidopsis. Plant Cell 24 (12), 5007-5023.
[95] Liu, X., Yang, Z., Wang, Y., Shen, Y., Jia, Q., Zhao, C., et al., 2022. Multiple caleosins have overlapping functions in oil accumulation and embryo development. J Exp Bot 73 (12), 3946-3962.
[96] Li, T., Xiong, Z., 2004. A novel response of wild-type duckweed (Lemna paucicostata Hegelm.) to heavy metals. Environ Toxicol: Int J 19 (2), 95-102.
[97] Elloumi, N., Zouari, M., Chaari, L., Jomni, C., Marzouk, B., Ben Abdallah, F., 2014. Effects of cadmium on lipids of almond seedlings (Prunus dulcis). Bot Stud 55, 1-9.
[98] Ge, S., Liu, D., Chu, M., Liu, X., Wei, Y., Che, X., et al., 2022. Dynamic and adaptive membrane lipid remodeling in leaves of sorghum under salt stress. Crop J 10 (6), 1557-1569.
[99] K\ddot{ }orber, T. T., Sitz, T., Abdalla, M. A., Mühling, K. H., Rohn, S., 2023. LC-ESI-MS/MS analysis of sulfolipids and galactolipids in green and red lettuce (Lactuca sativa L.) as influenced by sulfur nutrition. Int J Mol Sci 24 (4), 3728.
[100] Kolesnikov, Y., Kretynin, S., Bukhonska, Y., Pokotylo, I., Ruelland, E., Martinec, J., et al., 2022. Phosphatidic acid in plant hormonal signaling: from target proteins to membrane conformations. Int J Mol Sci 23 (6), 3227.
[101] Yu, D., Boughton, B. A., Hill, C. B., Feussner, I., Roessner, U., Rupasinghe, T. W., 2020. Insights into oxidized lipid modification in barley roots as an adaptation mechanism to salinity stress. Front Plant Sci 11, 1.
[102] Zhao, X., Wei, Y., Zhang, J., Yang, L., Liu, X., Zhang, H., et al., 2021. Membrane lipids' metabolism and transcriptional regulation in maize roots under cold stress. Front Plant Sci 12, 639132.
[103] Higashi, Y., Okazaki, Y., Takano, K., Myouga, F., Shinozaki, K., Knoch, E., et al., 2018. HEAT INDUCIBLE LIPASE1 remodels chloroplastic monogalactosyldiacylglycerol by liberating α-linolenic acid in Arabidopsis leaves under heat stress. Plant Cell 30 (8), 1887-1905.
[104] Higashi, Y., Saito, K., 2019. Lipidomic studies of membrane glycerolipids in plant leaves under heat stress. Prog Lipid Res 75, 100990.
[105] Yu, L., Shen, W., Fan, J., Sah, S. K., Mavraganis, I., Wang, L., et al., 2023. A chloroplast diacylglycerol lipase modulates glycerolipid pathway balance in Arabidopsis. Plant J.
[106] Eastmond, P. J., 2006. SUGAR-DEPENDENT1 encodes a patatin domain triacylglycerol lipase that initiates storage oil breakdown in germinating Arabidopsis seeds. Plant Cell 18 (3), 665-675.
[107] Azeez, A., Parchuri, P., Bates, P. D., 2022. Suppression of Physaria fendleri SDP1 increased seed oil and Hydroxy fatty acid content while maintaining oil biosynthesis through triacylglycerol remodeling. Front Plant Sci 13, 1861.
[108] Aznar-Moreno, J. A., Mukherjee, T., Morley, S. A., Duressa, D., Kambhampati, S., Chu, K. L., et al., 2022. Suppression of SDP1 improves soybean seed composition by increasing oil and reducing undigestible oligosaccharides. Front Plant Sci 13, 863254.
[109] Song, H., Taylor, D. C., Zhang, M., 2023. Bioengineering of soybean oil and its impact on agronomic traits. Int J Mol Sci 24 (3), 2256.
[110] Hwang, J.-H., Yu, S.-i, Lee, B.-h, Lee, D.-H., 2020. Modulation of energy metabolism is important for low-oxygen stress adaptation in Brassicaceae species. Int J Mol Sci 21 (5), 1787.
[111] Shiraku, M. L., Magwanga, R. O., Cai, X., Kirungu, J. N., Xu, Y., Mehari, T. G., et al., 2021. Functional characterization of GhACX3 gene reveals its significant role in enhancing drought and salt stress tolerance in cotton. Front Plant Sci 12, 658755.
[112] Liu, H., Xin, W., Wang, Y., Zhang, D., Wang, J., Zheng, H., et al., 2022. An integrated analysis of the rice transcriptome and lipidome reveals lipid metabolism plays a central role in rice cold tolerance. BMC Plant Biol 22 (1), 1-16.
[113] Xiao, R., Youngjun, O., Zhang, X., Thi, N. N., Lu, H., Hwang, I., 2023. Osmotic stress-induced localisation switch of CBR1 from mitochondria to the endoplasmic reticulum triggers ATP production via β-oxidation to respond to osmotic shock. Plant, Cell Environ 46 (11), 3420-3432.
[114] Arya, G. C., Sarkar, S., Manasherova, E., Aharoni, A., Cohen, H., 2021. The plant cuticle: an ancient guardian barrier set against long-standing rivals. Front Plant Sci 12, 663165.
[115] Lee, S. B., Suh, M. C., 2022. Regulatory mechanisms underlying cuticular wax biosynthesis. J Exp Bot 73 (9), 2799-2816.
[116] Lewandowska, M., Keyl, A., Feussner, I., 2020. Wax biosynthesis in response to danger: its regulation upon abiotic and biotic stress. N Phytol 227 (3), 698-713.
[117] Samuels, L., Kunst, L., Jetter, R., 2008. Sealing plant surfaces: cuticular wax formation by epidermal cells. Annu Rev Plant Biol 59, 683-707.
[118] Barthlott, W., Neinhuis, C., 1997. Purity of the sacred lotus, or escape from contamination in biological surfaces. Planta 202, 1-8.
[119] Neinhuis, C., Barthlott, W., 1997. Characterization and distribution of water-repellent, self-cleaning plant surfaces. Ann Bot 79 (6), 667-677.
[120] Takahashi, H., Abo, C., Suzuki, H., Romsuk, J., Oi, T., Yanagawa, A., et al., 2022. Triterpenoids in aerenchymatous phellem contribute to internal root aeration and waterlogging adaptability in soybeans. N Phytol.
[121] Kosma, D. K., Bourdenx, B., Bernard, A., Parsons, E. P., Lu, S., Joubes, J., et al., 2009. The impact of water deficiency on leaf cuticle lipids of Arabidopsis. Plant Physiol 151 (4), 1918-1929.
[122] Labudda, M., Dziurka, K., Fidler, J., Gietler, M., Rybarczyk-Pło\acute{ }nska, A., Nykiel, M., et al., 2022. The alleviation of metal stress nuisance for plants—a review of promising solutions in the face of environmental challenges. Plants 11 (19), 2544.
[123] Cui, F., Brosch\acute{ }e, M., Lehtonen, M. T., Amiryousefi, A., Xu, E., Punkkinen, M., et al., 2016. Dissecting abscisic acid signaling pathways involved in cuticle formation. Mol Plant 9 (6), 926-938.
[124] Dong, S., Beckles, D. M., 2019. Dynamic changes in the starch-sugar interconversion within plant source and sink tissues promote a better abiotic stress response. J Plant Physiol 234 80-93.
[125] Loka, D., Harper, J., Humphreys, M., Gasior, D., Wootton-Beard, P., Gwynn-Jones, D., et al., 2019. Impacts of abiotic stresses on the physiology and metabolism of cool-season grasses: A review. Food Energy Secur 8 (1), e00152.
[126] Peng, X., Teng, L., Yan, X., Zhao, M., Shen, S., 2015. The cold responsive mechanism of the paper mulberry: decreased photosynthesis capacity and increased starch accumulation. BMC Genom 16 (1), 1-20.
[127] Skirycz, A., De Bodt, S., Obata, T., De Clercq, I., Claeys, H., De Rycke, R., et al., 2010. Developmental stage specificity and the role of mitochondrial metabolism in the response of Arabidopsis leaves to prolonged mild osmotic stress. Plant Physiol 152 (1), 226-244.
[128] Yang, X., Liu, R., Jing, M., Zhang, N., Liu, C., Yan, J., 2023. Variation of root soluble sugar and starch response to drought stress in foxtail millet. Agronomy 13 (2), 359.
[129] Figueroa, C. M., Asencion Diez, M. D., Ballicora, M. A., Iglesias, A. A., 2022. Structure, function, and evolution of plant ADP-glucose pyrophosphorylase. Plant Mol Biol 108 (4-5), 307-323.
[130] Thalmann, M., Pazmino, D., Seung, D., Horrer, D., Nigro, A., Meier, T., et al., 2016. Regulation of leaf starch degradation by abscisic acid is important for osmotic stress tolerance in plants. Plant Cell 28 (8), 1860-1878.
[131] Zanella, M., Borghi, G. L., Pirone, C., Thalmann, M., Pazmino, D., Costa, A., et al., 2016. β-amylase 1 (BAM1) degrades transitory starch to sustain proline biosynthesis during drought stress. J Exp Bot 67 (6), 1819-1826.
[132] Lee, S. W., Choi, D., Moon, H., Kim, S., Kang, H., Paik, I., et al., 2023. PHYTOCHROME-INTERACTING FACTORS are involved in starch degradation adjustment via inhibition of the carbon metabolic regulator QUA-QUINE STARCH in Arabidopsis. Plant J 114 (1), 110-123.
[133] Baillo, E. H., Kimotho, R. N., Zhang, Z., Xu, P., 2019. Transcription factors associated with abiotic and biotic stress tolerance and their potential for crops improvement. Genes 10 (10), 771.
[134] Ma, Z., Hu, L., Jiang, W., 2024. Understanding AP2/ERF transcription factor responses and tolerance to various abiotic stresses in plants: a comprehensive review. Int J Mol Sci 25 (2), 893.
[135] Zhang, J., Zhao, H., Chen, L., Lin, J., Wang, Z., Pan, J., et al., 2023. Multifaceted roles of WRKY transcription factors in abiotic stress and flavonoid biosynthesis. Front Plant Sci 14.
[136] Guo, J., Sun, B., He, H., Zhang, Y., Tian, H., Wang, B., 2021. Current understanding of bHLH transcription factors in plant abiotic stress tolerance. Int J Mol Sci 22 (9), 4921.
[137] Liu, X., Chu, Z., 2015. Genome-wide evolutionary characterization and analysis of bZIP transcription factors and their expression profiles in response to multiple abiotic stresses in Brachypodium distachyon. BMC Genom 16 (1), 1-15.
[138] Riechmann, J. L., Meyerowitz, E. M., 1998. The AP2/EREBP family of plant transcription factors. Biol Chem 379, 633-646.
[139] Xie, Z., Nolan, T. M., Jiang, H., Yin, Y., 2019. AP2/ERF transcription factor regulatory networks in hormone and abiotic stress responses in Arabidopsis. Front Plant Sci 10, 228.
[140] Youm, J. W., Jeon, J. H., Choi, D., Yi, S. Y., Joung, H., Kim, H. S., 2008. Ectopic expression of pepper CaPF1 in potato enhances multiple stresses tolerance and delays initiation of in vitro tuberization. Planta 228, 701-708.
[141] Djemal, R., Khoudi, H., 2022. The ethylene-responsive transcription factor of durum wheat, TdSHN1, confers cadmium, copper, and zinc tolerance to yeast and transgenic tobacco plants. Protoplasma 259 (1), 19-31.
[142] Dixit, A., Tomar, P., Vaine, E., Abdullah, H., Hazen, S., Dhankher, O. P., 2018. A stress-associated protein, AtSAP13, from Arabidopsis thaliana provides tolerance to multiple abiotic stresses. Plant, Cell Environ 41 (5), 1171-1185.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed is:

1. A plant cell, plant seed, or plant comprising an expression system comprising an expression cassette comprising a promoter operably linked to a nucleic acid segment encoding a polypeptide for SpWRI3.

2. The plant cell, plant seed, or plant of claim 1, wherein the polypeptide for SpWRI3 has at least 95% sequence identity to SEQ ID NO: 1.

3. The plant cell, plant seed, or plant of claim 1, wherein the plant cell, plant seed, or plant is Spirodella polyrhiza, Arabadopsis thaliana, oilseed crop, perennial grass, bioenergy crop, cereal crop, horticulture plant, floriculture crop, biomass crop, vegetable crop, fruit crop, medicinal plant or tree species.

4. The plant cell, plant seed, or plant of claim 3, wherein the oilseed crop plant is a Brassica species, camelina, soybean, corn, sunflower, cotton, peanut, avocado, mustard, coconut, sesamum, caster seeds, linseed, oil palm, olive, jatropha, camelina, pennycress, winter rye, sweet potato, poplar tree, rice, or tung nut tree.

5. The plant cell, plant seed, or plant of claim 1, wherein the promoter is a strong constitutive or inducible promoter.

6. The plant cell, plant seed, or plant of claim 1, wherein the promoter is a tissue-specific promoter.

7. The plant cell, plant seed, or plant of claim 1, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter, and Arabidopsis Ubiquitin 10 promoter.

8. The plant cell, plant seed, or plant of claim 1, wherein the expression of the promoter operably linked to the nucleic acid segment encoding the polypeptide for SpWRI3 increases the heating value of the plant cell, plant seed, or plant relative to a plant cell, plant seed, or plant that does not express the promoter operably linked to the nucleic acid segment encoding the polypeptide for SpWRI3.

9. An expression cassette comprising a promoter operably linked to a nucleic acid segment encoding a polypeptide for SpWRI3.

10. The expression cassette of claim 9, wherein the polypeptide for SpWRI3 has at least 95% sequence identity to SEQ ID NO: 1.

11. The expression cassette of claim 9, wherein the promoter is selected from a cauliflower mosaic virus promoter (such as CaMV 35S or CaMV 19S), nos promoter, Adh1 promoter, sucrose synthase promoter, α-tubulin promoter, ubiquitin promoter, actin promoter (such as from rice), cab promoter, PEPCase promoter, R gene complex promoter, poplar xylem-specific secondary cell wall specific cellulose synthase 8 promoter, Z10 promoter from a gene encoding a 10 kDa zein protein, Z27 promoter from a gene encoding a 27 kDa zein protein, pea rbcS gene, and phaseolin promoter, and Arabidopsis Ubiquitin 10 promoter

12. The expression cassette of claim 9, wherein the promoter is a strong or inducible promoter.

13. The expression cassette of claim 9, wherein the promoter is a tissue-specific promoter.

14. A method of growing a plant seed or plant comprising:
introducing into at least one plant cell at least one transgene or expression cassette encoding a polypeptide for SpWRI3 to generate one or more transformed plant cells; and
generating a plant from one or more transformed plant cell(s),
wherein the plant seed or plant is tolerant to heavy metal stress, salt stress, drought or a combination thereof.

15. The method of claim 14, wherein the polypeptide for SpWRI3 has at least 95% sequence identity to SEQ ID NO: 1.

16. The method of claim 14, further comprising harvesting triacylglycerol (TAG), TAG enriched with omega-3 fatty acids, carbohydrates, or a combination thereof.

17. The method of claim 16, wherein the carbohydrates comprise a starch with increased starch granule size, glucose, sucrose, or a combination thereof.

18. The method of claim 14, further comprising contacting the plant seed or plant with flue gas desulfurization (FGD) wastewater while generating the plant from the one or more transformed plant cell(s).

19. The method of claim 14, further comprising contacting the plant seed or plant with a marginal soil while generating the plant from the one or more transformed plant cell(s).

20. The method of claim 19, wherein the marginal soil is a reclaimed coal mine soil or other soil contaminated with industrial chemical or heavy metals.

21. The method of claim 14, wherein the plant seed or plant is generated in salt stress conditions.

22. The method of claim 14, wherein the plant seed or plant is Spirodella polyrhiza, Arabadopsis thaliana, or an oilseed crop plant.

23. The method of claim 22, wherein the Spirodella polyrhiza and Arabidopsis thaliana is grown hydroponically.
